(19) United States
(12) Patent Application Publication
(10) Pub. No.: US 2015/0065524 A1
(43) Pub. Date: Mar. 5, 2015

(54) KINASE INHIBITOR POLYMORPHS

(71) Applicants: Pingda Ren, San Diego, CA (US); Michael Martin, San Marcos, CA (US)

(72) Inventors: Pingda Ren, San Diego, CA (US); Michael Martin, San Marcos, CA (US)

(73) Assignee: Intellikine LLC, La Jolla, CA (US)

(21) Appl. No.: 14/238,426

(22) PCT Filed: Aug. 10, 2012
(86) PCT No.: PCT/US12/50453

§ 371 Date: Oct. 17, 2014

Related U.S. Application Data
(60) Provisional application No. 61/522,624, filed on Aug. 11, 2011.

(51) Int. Cl. C07D 471/04

(52) U.S. Cl. CPC C07D 471/04; C07B 2200/13
USPC 514/262.1; 544/262

(57) ABSTRACT

Polymorphs, hydrates, and solvates of chemical compounds that modulate kinase activity, including mTOR activity, and chemical compounds, pharmaceutical compositions, and methods of treatment of diseases and conditions associated with kinase activity, including mTOR activity, are described herein.

This application claims priority to U.S. Provisional Application 61/522,624, filed Aug. 11, 2011, the contents of which are incorporated by reference in their entirety.

### BACKGROUND OF THE INVENTION

PI3Ks constitute a unique and conserved family of intracellular lipid kinases that phosphorylate the 3′-OH group on phosphatidylinositols or phosphoinositides. The PI3K family comprises 15 kinases with distinct substrate specificities, expression patterns, and modes of regulation. The class I PI3Ks (p110α, p110β, p110δ, and p110γ) are typically activated by tyrosine kinases or G-protein coupled receptors to generate a lipid product termed PIP₃, which engages downstream effectors such as those in the Akt/PDK1 pathway, mTOR, the Tec family kinases, and the Rho family GTPases. The class II and III PI3-Ks play a key role in intracellular trafficking through the synthesis of PI(3)P and PI(3,4)P2.

mTOR is a serine-threonine kinase related to the lipid kinases of the PI3K family mTOR has been implicated in a wide range of biological processes including cell growth, cell proliferation, cell motility and survival. Disregulation of the mTOR pathway has been reported in various types of cancer. mTOR is a multifunctional kinase that integrates growth factor and nutrient signals to regulate protein translation, nutrient uptake, autophagy, and mitochondrial function.

mTOR exists in two complexes, mTORC1 and mTORC2. mTORC1 contains the raptor subunit and mTORC2 contains rictor. These complexes are differentially regulated, and have distinct substrate specificities and rapamycin sensitivity. For example, mTORC1 phosphorylates S6 kinase (S6K) and 4EBP1, promoting increased translation and ribosome biogenesis to facilitate cell growth and cell cycle progression. S6K also acts in a feedback pathway to attenuate PI3K/Akt activation. mTORC2 is generally insensitive to rapamycin. mTORC2 is though to modulate growth factor signaling by phosphorylating the C-terminal hydrophobic motif of some AGC kinases such as Akt. In many cellular contexts, mTORC2 is required for phosphorylation of the S473 site of Akt.

Over the past decade, mTOR has drawn considerable attention due to its role in cell growth control and its involvement in human diseases. mTor has been implicated in a wide range of disorders including but not limited to cancer, diabetes, obesity, cardiovascular diseases and neurological disorders. It has been shown that mTOR modulates many fundamental biological processes including transcription, translation, autophagy, actin organization and ribosome biogenesis by integrating intracellular and extracellular signals, such as signals mediated by growth factors, nutrients, energy levels and cellular stress.

As such, kinases particularly protein kinases such as mTor and Akt, as well as lipid kinases such as PI3Ks are prime targets for drug development. While compounds with inhibitory activity of such targets are often initially evaluated for their activity when dissolved in solution, solid state characteristics such as polymorphism are also important. Polymorphic forms of a drug substance such as an inhibitor of mTOR can have different chemical and physical properties, including melting point, chemical reactivity, apparent solubility, dissolution rate, optical and mechanical properties, vapor pressure, and density. These properties can have a direct effect on the ability to process or manufacture a drug substance and the drug product. Moreover, polymorphism is often a factor under regulatory review of the ‘sameness’ of drug products from various manufacturers. For example, polymorphism has been evaluated in many multi-million dollar and even multi-billion dollar drugs, such as warfarin sodium, famotidine, and ranitidine. Polymorphism can affect the quality, safety, and/or efficacy of a drug product, such as a kinase inhibitor.

Thus, there still remains a need for polymorphs of inhibitors of mTor and/or Akt, as well as lipid kinases such as PI3Ks. This invention addresses this need and provides related advantages as well.

### SUMMARY OF THE INVENTION

In one embodiment, the invention is directed to a method of making polymorph Form C of the compound of Formula I:

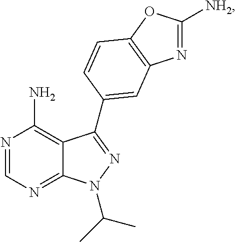 [SMILES: CC(C)n1nc(-c2ccc3oc(N)nc3c2)c2c(N)ncnc21]

where the method comprises (i) exposing a composition comprising one or more non-Form C polymorphs of the compound of Formula I to non-anhydrous conditions for a period of time sufficient to convert at least about 50% of the total amount of non-Form C polymorphs into Form C of the compound of Formula I; and (ii) isolating said polymorph Form C. In various embodiments, the non-anhydrous conditions include water in a form selected from water vapor and liquid water. The non-anhydrous conditions may include a binary crystallization system comprising a non-water solvent and liquid water. In various embodiments, the non-water solvent is dioxane or THF. For example, the liquid water may be present in an amount selected from about 1%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95, and 100% by volume of the solvent system. In various embodiments, liquid water is present in an amount between about 10% to about 50% by volume of the solvent system.

Non-Form C polymorphs may be selected from the group consisting of Form A, Form B, Form D, Form E, Form F, amorphous form, and mixtures thereof. For example, the one or more non-Form C polymorphs may comprise at least 50% by weight polymorph Form A. In various embodiments, one or more non-C Forms are obtained from crude or purified Form C.

In one embodiment, the invention is directed to a method of making polymorph Form C of the compound of Formula I:

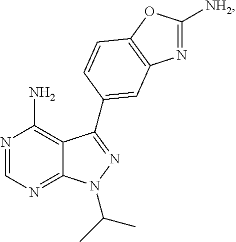 [SMILES: CC(C)n1nc(-c2ccc3oc(N)nc3c2)c2c(N)ncnc21]

said method comprising (i) reacting compounds 2 and 5:

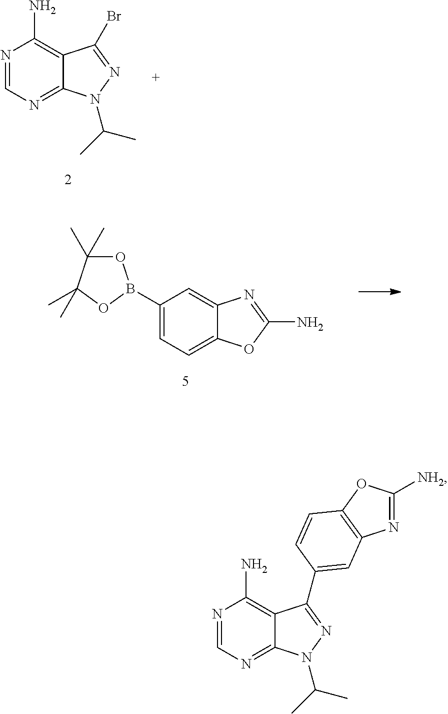 [SMILES: CC(C)n1nc(Br)c2c(N)ncnc21 ; CC1(C)OB(c2ccc3oc(N)nc3c2)OC1(C)C ; CC(C)n1nc(-c2ccc3oc(N)nc3c2)c2c(N)ncnc21]

or reacting compounds 2 and 5a:

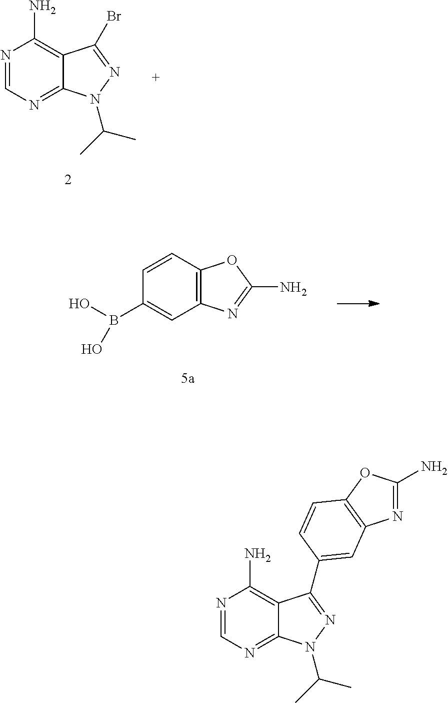 [SMILES: CC(C)n1nc(Br)c2c(N)ncnc21 ; Nc1nc2cc(B(O)O)ccc2o1 ; CC(C)n1nc(-c2ccc3oc(N)nc3c2)c2c(N)ncnc21]

to yield a compound of Formula I; and (ii) isolating said compound of Formula I in polymorph Form C; wherein at least one of steps (i) and (ii) occurs in non-anhydrous conditions. For example, the non-anhydrous conditions may include water vapor and/or liquid water. Compound 5a can be a salt, such as the HCl salt, or internal salt, or a non-salt form.

In various embodiments, the invention is directed to a method of making polymorph Form A of the compound of Formula I:

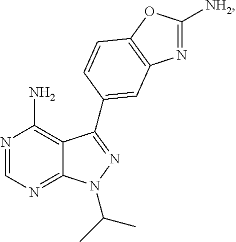 [SMILES: CC(C)n1nc(-c2ccc3oc(N)nc3c2)c2c(N)ncnc21]

the method comprising reacting compounds 2 and 5:

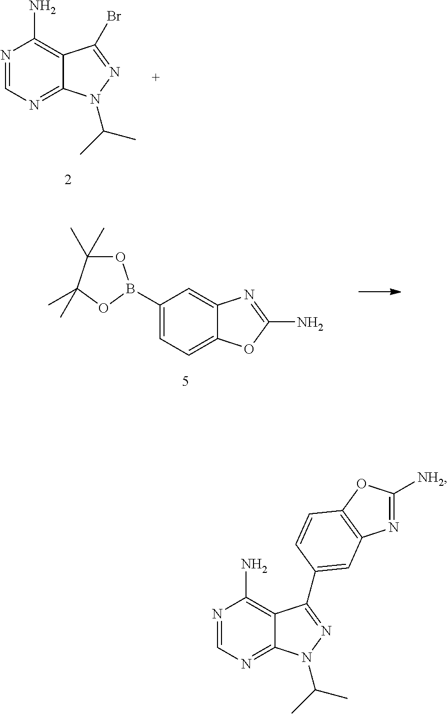 [SMILES: CC(C)n1nc(Br)c2c(N)ncnc21 ; CC1(C)OB(c2ccc3oc(N)nc3c2)OC1(C)C ; CC(C)n1nc(-c2ccc3oc(N)nc3c2)c2c(N)ncnc21]

or reacting compounds 2 and 5a

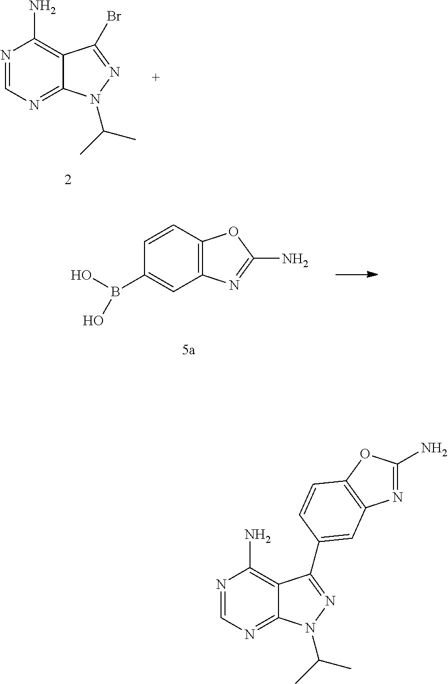 [SMILES: CC(C)n1nc(Br)c2c(N)ncnc21 ; Nc1nc2cc(B(O)O)ccc2o1 ; CC(C)n1nc(-c2ccc3oc(N)nc3c2)c2c(N)ncnc21]

to yield a compound of Formula I; and (ii) isolating the compound of Formula I in polymorph Form A. Compound 5a can be a salt, such as the HCl salt, or internal salt, or a non-salt form. Step (ii) may include recrystallization of the compound of Formula I from either a mono-solvent system, or from a multi-solvent system. In various embodiments, the invention includes a step of dissolving the compound of Formula I in a solvent or solvents, removing residual solid matter to yield a liquid solution, actively cooling said liquid solution at a rate to effect crystallization of Form A, and separating Form A from the liquid solution. In various embodiments, the compound of Formula I is treated to remove palladium, for example with activated charcoal in methanol.

In various embodiments, the invention is directed to a pharmaceutically acceptable salt of the compound of Formula I:

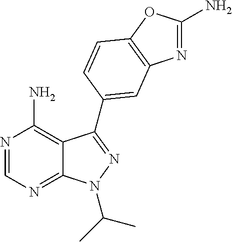 [SMILES: CC(C)n1nc(-c2ccc3oc(N)nc3c2)c2c(N)ncnc21]

and/or solvate thereof. In various embodiments, the salt (mono or bis) is selected from L-tartaric acid, p-toluenesulfonic acid, D-glucaronic acid, ethane-1,2-disulfonic acid (EDSA), 2-naphthalenesulfonic acid (NSA), hydrochloric acid (HCl) (mono and bis), hydrobromic acid (HBr), citric acid, naphthalene-1,5-disulfonic acid (NDSA), DL-mandelic acid, fumaric acid, sulfuric acid, maleic acid, methanesulfonic acid (MSA), benzenesulfonic acid (BSA), ethanesulfonic acid (ESA), L-malic acid, phosphoric acid, and aminoethanesulfonic acid (taurine). The compound may be the HCl salt or the bis-HCl salt.

In various embodiments, the invention is directed to a composition comprising the compound of Formula I:

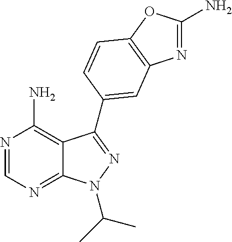 [SMILES: CC(C)n1nc(-c2ccc3oc(N)nc3c2)c2c(N)ncnc21]

or a pharmaceutically acceptable salt and/or solvate thereof, where the composition comprises a mixture of polymorph Form C and one or more non-C polymorphs. For example, the composition may comprise polymorph Form C and polymorph Form A. In various embodiments, the ratio of polymorph Form C to the total amount of non-C polymorphs is greater than about 1:1, or greater than about 9:1. In various embodiments, the composition is at least 98% by weight compound of Formula I.

In various embodiments, the compound of Formula I is modified by replacing one or more hydrogen atoms with deuterium atoms. In various embodiments, the one or more hydrogen atoms to be replaced are selected from hydrogens attached to a carbon atom, for example, as represented by H1-H11 in the following formula:

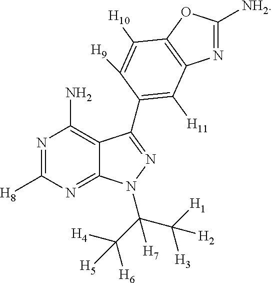

In various embodiments, the invention is directed to a composition comprising a therapeutically effective amount of the compound of Formula I:

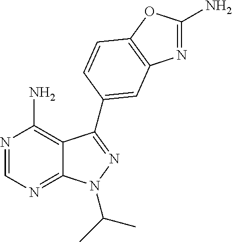 [SMILES: CC(C)n1nc(-c2ccc3oc(N)nc3c2)c2c(N)ncnc21]

or a pharmaceutically acceptable salt and/or solvate thereof, and a pharmaceutically acceptable carrier; where the composition comprises polymorph Form C of the compound of Formula I. The composition may further comprise one or more non-C polymorphs of the compound of Formula I. In various embodiments, the ratio of polymorph Form C to the total amount of non-C polymorphs is greater than about 1:1.

Compositions may be in a solid dosage form. In various embodiments, a pharmaceutical composition comprises compounds of Formula I and III,

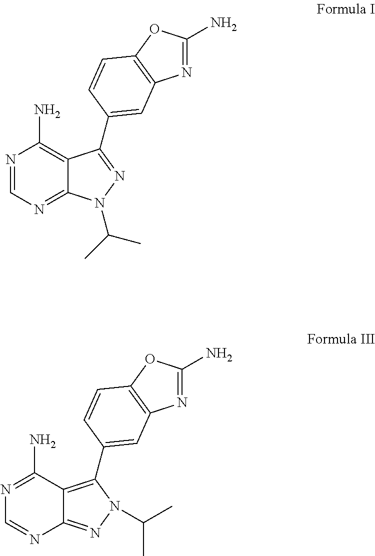 [SMILES: CC(C)n1nc(-c2ccc3oc(N)nc3c2)c2c(N)ncnc21 ; CC(C)n1nc2ncnc(N)c2c1-c1ccc2oc(N)nc2c1]

wherein the amount of compound of Formula III is less than 50% by weight, less than 40% by weight, less than 30% by weight, less than 20% by weight, less than 10% by weight, less than 5% by weight, less than 4% by weight, less than 3% by weight, less than 2% by weight, less than 1% by weight, less than 0.1% by weight, or less than 0.01% by weight, all amounts being about, with respect to the amount of Formula I.

In various embodiments, the invention is directed to a composition comprising a therapeutically effective amount of the compound of Formula I:

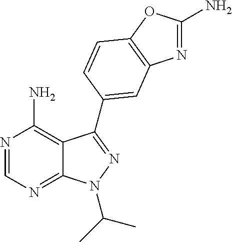 [SMILES: CC(C)n1nc(-c2ccc3oc(N)nc3c2)c2c(N)ncnc21]

or a pharmaceutically acceptable salt and/or solvate thereof, and a pharmaceutically acceptable carrier; where the composition comprises a solvate or hydrate of the compound of Formula I. The composition may comprise one or more polymorphs of the compound of Formula I in hydrated or solvated form. In various embodiments, the hydrate is a hydrate of Form A. In various embodiments, the solvate is a solvate of Form A. In various embodiments, the solvate is a dimethylacetamide (DMA) solvate.

Compositions according to the invention may be used for the treatment of an mTOR-associated disorder, where the method comprises administering the composition to an individual in need thereof.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### DESCRIPTION OF THE DRAWINGS

The novel features of the invention are set forth with particularity in the appended claims. An understanding of the features and advantages of the present invention may be obtained by reference to the following detailed description that sets forth illustrative embodiments, in which the principles of the invention are utilized, and the accompanying drawings of which:

FIG. 1 shows a high-resolution XRPD diffractogram of polymorph Form A.

FIG. 2 shows an XRPD diffractogram of polymorph Form A before and after storing at 40° C./75% RH for 1 week, 3 weeks, and 5 weeks.

FIG. 3 shows a TGA trace of polymorph Form A.

FIG. 4 shows a DSC trace of polymorph Form A.

FIG. 5 shows a GVS kinetic plot of polymorph Form A.

FIG. 6 shows XRPD patterns for polymorph Form B.

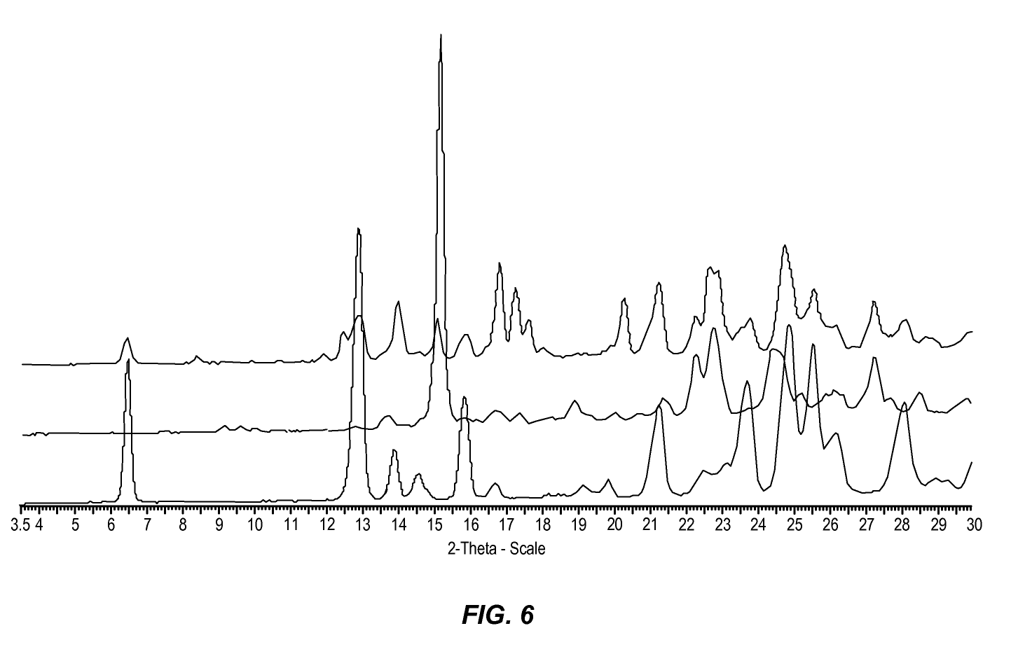

FIG. 7 shows a DSC trace for polymorph Form B.

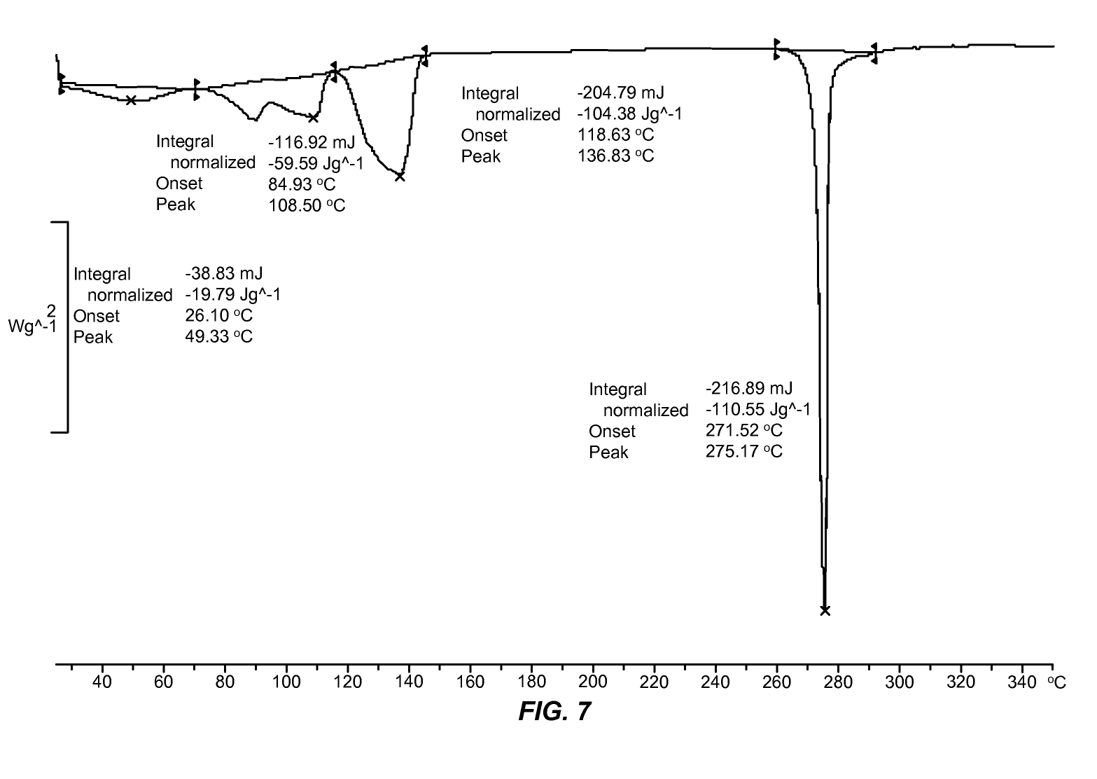

FIG. 8 shows XRPD patterns for polymorph Form C and Form D.

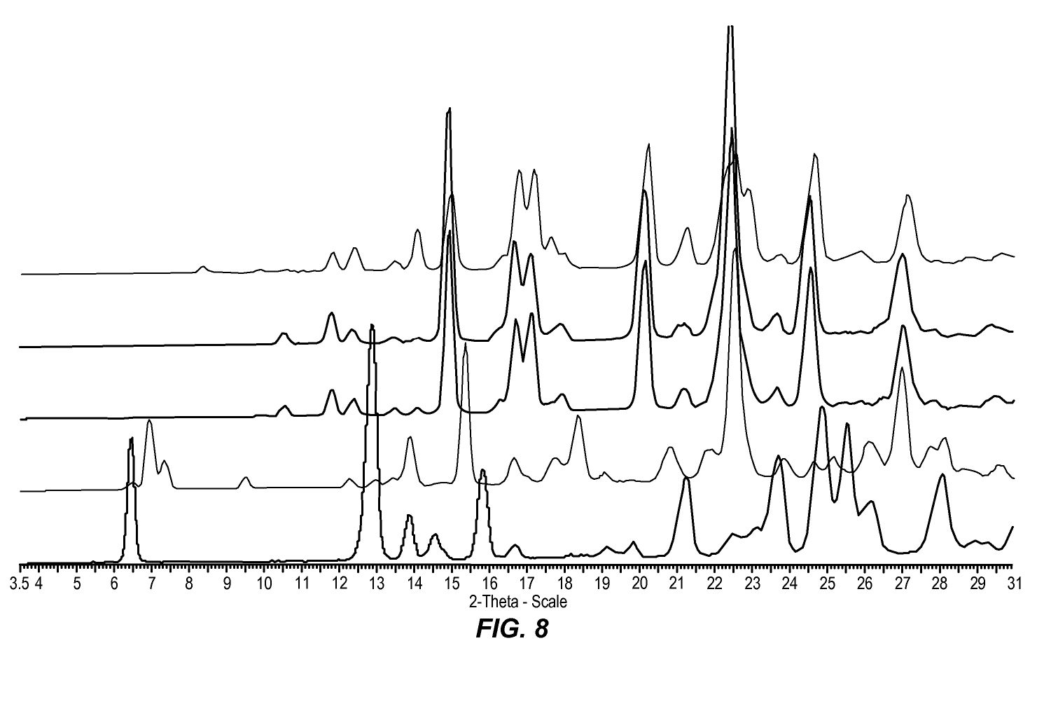

FIG. 9 shows a TGA trace for polymorph Form C.

FIG. 10 shows a DSC trace for polymorph Form C.

FIG. 11 shows a DSC trace for polymorph Form D.

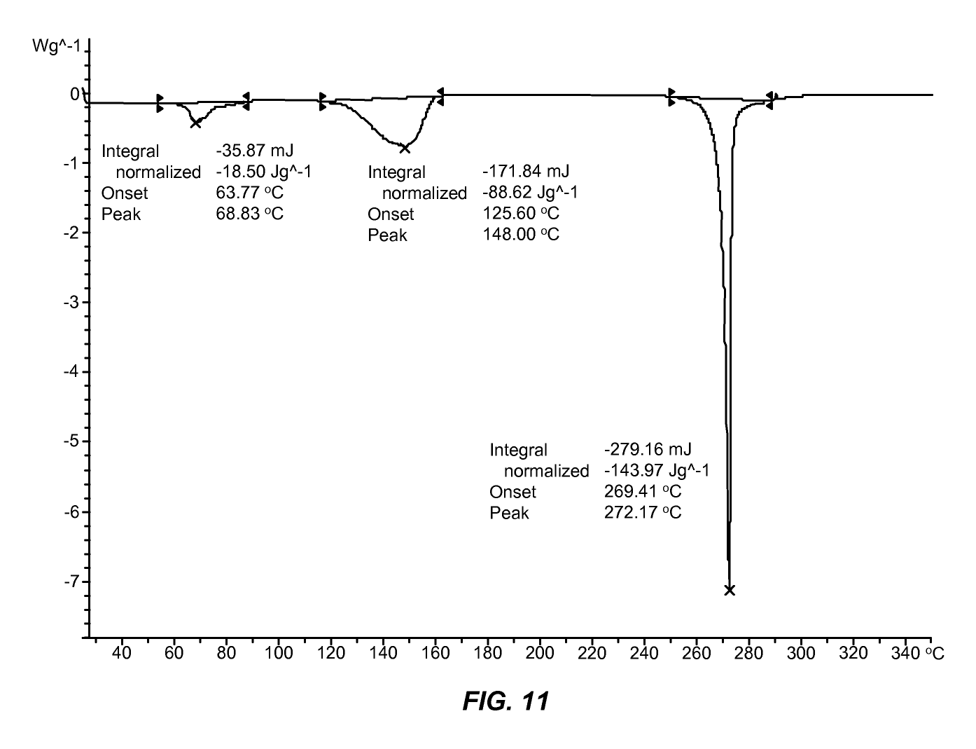

FIG. 12 shows XRPD patterns for polymorph Forms A, B, C and E from a scale-up experiment.

FIG. 13 shows a TGA trace of polymorph Form E.

FIG. 14 shows a GVS trace of polymorph Form C.

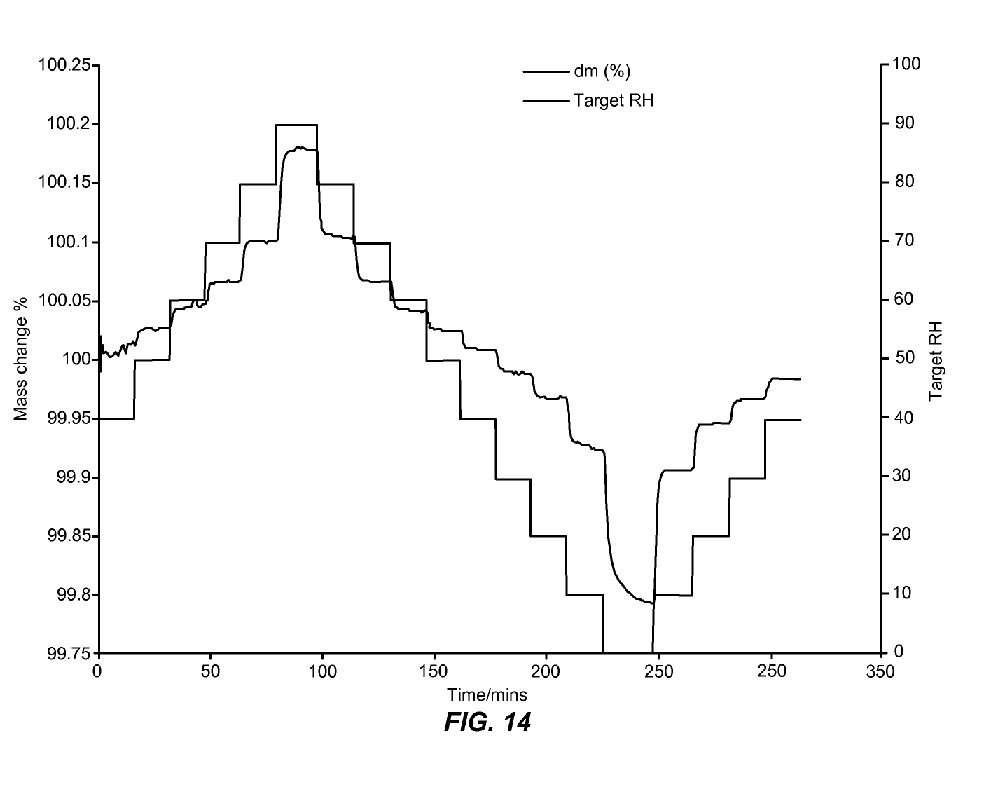

FIG. 15 shows an XRPD diffractogram of a hydrate of Formula I.

FIG. 16 shows a TGA trace and a DSC trace of a hydrate of Formula I.

FIG. 17 shows an XRPD diffractogram of a dimethylacetamide (DMA) solvate of Formula I.

FIG. 18 shows a TGA trace and a DSC trace of a DMA solvate of Formula I.

### DETAILED DESCRIPTION OF THE INVENTION

While preferred embodiments of the present invention have been shown and described herein, it will be apparent to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the appended claims define the scope of the invention and that methods and structures within the scope of these claims and their equivalents be covered thereby.

### I. Definitions

Unless defined otherwise, all technical and scientific terms used herein have the same meaning as is commonly understood by one of skill in the art to which this invention belongs.

As used in the specification and claims, the singular form “a”, “an” and “the” includes plural references unless the context clearly dictates otherwise.

As used herein, “agent” or “biologically active agent” refers to a biological, pharmaceutical, or chemical compound or other moiety. Non-limiting examples include simple or complex organic or inorganic molecule, a peptide, a protein, an oligonucleotide, an antibody, an antibody derivative, antibody fragment, a vitamin derivative, a carbohydrate, a toxin, or a chemotherapeutic compound. Various compounds can be synthesized, for example, small molecules and oligomers (e.g., oligopeptides and oligonucleotides), and synthetic organic compounds based on various core structures. In addition, various natural sources can provide compounds for screening, such as plant or animal extracts, and the like. A skilled artisan can readily recognize that there is no limit as to the structural nature of the agents of the present invention.

The term “agonist” as used herein refers to a compound having the ability to initiate or enhance a biological function of a target protein, whether by inhibiting the activity or expression of the target protein. Accordingly, the term “agonist” is defined in the context of the biological role of the target polypeptide. While preferred agonists herein specifically interact with (e.g. bind to) the target, compounds that initiate or enhance a biological activity of the target polypeptide by interacting with other members of the signal transduction pathway of which the target polypeptide is a member are also specifically included within this definition.

The terms “antagonist” and “inhibitor” are used interchangeably, and they refer to a compound having the ability to inhibit a biological function of a target protein, whether by inhibiting the activity or expression of the target protein. Accordingly, the terms “antagonist” and “inhibitors” are defined in the context of the biological role of the target protein. While preferred antagonists herein specifically interact with (e.g. bind to) the target, compounds that inhibit a biological activity of the target protein by interacting with other members of the signal transduction pathway of which the target protein is a member are also specifically included within this definition. A preferred biological activity inhibited by an antagonist is associated with the development, growth, or spread of a tumor, or an undesired immune response as manifested in autoimmune disease.

An “anti-cancer agent”, “anti-tumor agent” or “chemotherapeutic agent” refers to any agent useful in the treatment of a neoplastic condition. One class of anti-cancer agents comprises chemotherapeutic agents. “Chemotherapy” means the administration of one or more chemotherapeutic drugs and/or other agents to a cancer patient by various methods, including intravenous, oral, intramuscular, intraperitoneal, intravesical, subcutaneous, transdermal, buccal, or inhalation or in the form of a suppository.

The term “cell proliferation” refers to a phenomenon by which the cell number has changed as a result of division. This term also encompasses cell growth by which the cell morphology has changed (e.g., increased in size) consistent with a proliferative signal.

The term “co-administration,” “administered in combination with,” and their grammatical equivalents, as used herein, encompasses administration of two or more agents to an animal so that both agents and/or their metabolites are present in the animal at the same time. Co-administration includes simultaneous administration in separate compositions, administration at different times in separate compositions, or administration in a composition in which both agents are present.

The term “effective amount” or “therapeutically effective amount” refers to that amount of a compound described herein that is sufficient to effect the intended application including but not limited to disease treatment, as defined below. The therapeutically effective amount may vary depending upon the intended application (in vitro or in vivo), or the subject and disease condition being treated, e.g., the weight and age of the subject, the severity of the disease condition, the manner of administration and the like, which can readily be determined by one of ordinary skill in the art. The term also applies to a dose that will induce a particular response in target cells, e.g. reduction of platelet adhesion and/or cell migration. The specific dose will vary depending on the particular compounds chosen, the dosing regimen to be followed, whether it is administered in combination with other compounds, timing of administration, the tissue to which it is administered, and the physical delivery system in which it is carried.

As used herein, the terms “treatment”, “treating”, “palliating” and “ameliorating” are used interchangeably herein. These terms refer to an approach for obtaining beneficial or desired results including but not limited to therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient may still be afflicted with the underlying disorder. For prophylactic benefit, the compositions may be administered to a patient at risk of developing a particular disease, or to a patient reporting one or more of the physiological symptoms of a disease, even though a diagnosis of this disease may not have been made.

A “therapeutic effect,” as that term is used herein, encompasses a therapeutic benefit and/or a prophylactic benefit as described above. A prophylactic effect includes delaying or eliminating the appearance of a disease or condition, delaying or eliminating the onset of symptoms of a disease or condition, slowing, halting, or reversing the progression of a disease or condition, or any combination thereof.

The term “pharmaceutically acceptable salt” refers to salts derived from a variety of organic and inorganic counter ions well known in the art. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, tetraalkylammonium, calcium, magnesium, iron, zinc, copper, manganese, aluminum, and the like. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. In some embodiments, the pharmaceutically acceptable base addition salt is chosen from ammonium, potassium, sodium, calcium, and magnesium salts. Bis salts (i.e. two counterions), tris salts, and higher salts are encompassed within the meaning of pharmaceutically acceptable salts.

“Pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions of the invention is contemplated. Supplementary active ingredients can also be incorporated into the compositions.

“Subject” refers to an animal, such as a mammal, for example a human. The methods described herein can be useful in both human therapeutics and veterinary applications. In some embodiments, the patient is a mammal, and in some embodiments, the patient is human.

“Prodrug” is meant to indicate a compound that may be converted under physiological conditions or by solvolysis to a biologically active compound described herein. Thus, the term “prodrug” refers to a precursor of a biologically active compound that is pharmaceutically acceptable. A prodrug may be inactive when administered to a subject, but is converted in vivo to an active compound, for example, by hydrolysis. The prodrug compound often offers advantages of solubility, tissue compatibility or delayed release in a mammalian organism (see, e.g., Bundgard, H., Design of Prodrugs (1985), pp. 7-9, 21-24 (Elsevier, Amsterdam) A discussion of prodrugs is provided in Higuchi, T., et al., “Pro-drugs as Novel Delivery Systems,” A.C.S. Symposium Series, Vol. 14, and in Bioreversible Carriers in Drug Design, ed. Edward B. Roche, American Pharmaceutical Association and Pergamon Press, 1987, both of which are incorporated in full by reference herein. The term “prodrug” is also meant to include any covalently bonded carriers, which release the active compound in vivo when such prodrug is administered to a mammalian subject. Prodrugs of an active compound, as described herein, may be prepared by modifying functional groups present in the active compound in such a way that the modifications are cleaved, either in routine manipulation or in vivo, to the parent active compound. Prodrugs include compounds wherein a hydroxy, amino or mercapto group is bonded to any group that, when the prodrug of the active compound is administered to a mammalian subject, cleaves to form a free hydroxy, free amino or free mercapto group, respectively. Examples of prodrugs include, but are not limited to, acetate, formate and benzoate derivatives of an alcohol or acetamide, formamide and benzamide derivatives of an amine functional group in the active compound and the like.

The term “in vivo” refers to an event that takes place in a subject's body.

The term “in vitro” refers to an event that takes places outside of a subject's body. For example, an in vitro assay encompasses any assay run outside of a subject assay. In vitro assays encompass cell-based assays in which cells alive or dead are employed. In vitro assays also encompass a cell-free assay in which no intact cells are employed.

The term “isolating” also encompasses purifying.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of this invention.

The compounds of the present invention may also contain unnatural proportions of atomic isotopes at one or more of atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds of the present invention, whether radioactive or not, are encompassed within the scope of the present invention.

When ranges are used herein for physical properties, such as molecular weight, or chemical properties, such as chemical formulae, all combinations and subcombinations of ranges and specific embodiments therein are intended to be included. The term “about” when referring to a number or a numerical range means that the number or numerical range referred to is an approximation within experimental variability (or within statistical experimental error), and thus the number or numerical range may vary from, for example, between 1% and 15% of the stated number or numerical range. The term “comprising” (and related terms such as “comprise” or “comprises” or “having” or “including”) includes those embodiments, for example, an embodiment of any composition of matter, composition, method, or process, or the like, that “consist of” or “consist essentially of” the described features. The phrase “consists essentially of” excludes unnamed components which materially change the material or composition in major proportions and/or in trace amounts.

The terms “solvent,” “organic solvent,” or “inert solvent” each mean a solvent inert under the conditions of the reaction being described in conjunction therewith including, for example, benzene, toluene, acetonitrile, tetrahydrofuran (“THF”), dimethylformamide (“DMF”), chloroform, methylene chloride (or dichloromethane), diethyl ether, methanol, N-methylpyrrolidone (“NMP”), pyridine and the like. Unless specified to the contrary, the solvents used in the reactions described herein are inert organic solvents. Unless specified to the contrary, for each gram of a limiting reagent, one cc (or mL) of solvent constitutes a volume equivalent.

“Solvate” refers to a compound (e.g., a compound as described herein or a pharmaceutically acceptable salt thereof) in physical association with one or more molecules of a pharmaceutically acceptable solvent.

“Crystalline form,” “polymorph,” and “novel form” may be used interchangeably herein, and are meant to include all crystalline and amorphous forms of the compound, including, for example, polymorphs, pseudopolymorphs, solvates, hydrates, unsolvated polymorphs (including anhydrates), conformational polymorphs, and amorphous forms, as well as mixtures thereof, unless a particular crystalline or amorphous form is referred to. Compounds of the present invention include crystalline and amorphous forms of those compounds, including, for example, polymorphs, pseudopolymorphs, solvates, hydrates, unsolvated polymorphs (including anhydrates), conformational polymorphs, and amorphous forms of the compounds, as well as mixtures thereof.

Pharmaceutically acceptable forms of the compounds recited herein include pharmaceutically acceptable salts, chelates, non-covalent complexes, prodrugs, and mixtures thereof. In certain embodiments, the compounds described herein are in the form of pharmaceutically acceptable salts. Hence, the terms “chemical entity” and “chemical entities” also encompass pharmaceutically acceptable salts, chelates, non-covalent complexes, prodrugs, and mixtures.

In addition, if the compound of the invention is obtained as an acid addition salt, the free base can be obtained by basifying a solution of the acid salt. Conversely, if the product is a free base, an addition salt, particularly a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used to prepare non-toxic pharmaceutically acceptable addition salts.

### II. Compounds and Methods of Making

The chemical entities described herein can generally be synthesized by an appropriate combination of generally well known synthetic methods. Techniques useful in synthesizing these chemical entities are both readily apparent and accessible to those of skill in the relevant art, based on the instant disclosure. Many of the optionally substituted starting compounds and other reactants are commercially available, e.g., from Aldrich Chemical Company (Milwaukee, Wis.) or can be readily prepared by those skilled in the art using commonly employed synthetic methodology.

The polymorphs made according to the methods of the invention may be characterized by any methodology according to the art. For example, the polymorphs made according to the methods of the invention may be characterized by X-ray powder diffraction (XRPD), differential scanning calorimetry (DSC), thermogravimetric analysis (TGA), hot-stage microscopy, and spectroscopy (e.g., Raman, solid state nuclear magnetic resonance (ssNMR), and infrared (IR)).

XRPD

Polymorphs according to the invention may be characterized by X-ray powder diffraction patterns (XRPD). The relative intensities of XRPD peaks can vary, depending upon the sample preparation technique, the sample mounting procedure and the particular instrument employed. Moreover, instrument variation and other factors can affect the 2-0 values. Therefore, the XRPD peak assignments can vary by plus or minus about 0.2 degrees.

DSC

Polymorphs according to the invention can also be identified by its characteristic differential calorimeter scanning (DSC) trace such as shown in the figures. For DSC, it is known that the temperatures observed will depend upon the rate of temperature change as well as sample preparation technique and the particular instrument employed. Thus, the values reported herein relating to DSC thermograms can vary by plus or minus about 4° C.

TGA

The polymorphic forms of the invention may also give rise to thermal behavior different from that of the amorphous material or another polymorphic form. Thermal behavior may be measured in the laboratory by thermogravimetric analysis (TGA) which may be used to distinguish some polymorphic forms from others. In one aspect, the polymorph may be characterized by thermogravimetric analysis.

The polymorph forms of the invention are useful in the production of medicinal preparations and can be obtained by means of a crystallization process to produce crystalline and semi-crystalline forms or a solidification process to obtain the amorphous form. In various embodiments, the crystallization is carried out by either generating the compound of Formula I in a reaction mixture and isolating the desired polymorph from the reaction mixture, or by dissolving raw compound in a solvent, optionally with heat, followed by crystallizing/solidifying the product by cooling (including active cooling) and/or by the addition of an antisolvent for a period of time. The crystallization or solidification may be followed by drying carried out under controlled conditions until the desired water content is reached in the end polymorphic form.

In one aspect, the invention provides methods of making one or more polymorphs of the compound of the Formula I:

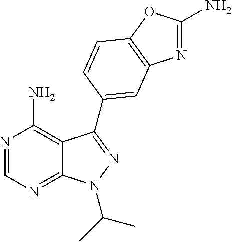 [SMILES: CC(C)n1nc(-c2ccc3oc(N)nc3c2)c2c(N)ncnc21]

or a pharmaceutically acceptable salt and/or solvate thereof. Polymorphs according to the methods of the invention can be selected from Form A, Form B, Form C, Form D, Form E, an amorphous form, and mixtures of more than one form. In addition, polymorphs made according to the invention may include solvates. In various embodiments, polymorphs of the invention are prepared as the free base, the mono-salt, or the bis-salt, such as the HCl salt or the bis-HCl salt of the compound of Formula I.

In various embodiments, the intermediates for the synthesis of Formula I are made according to the following schemes.

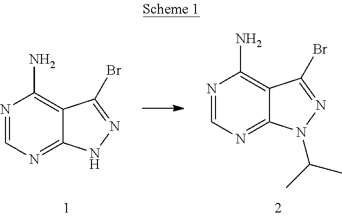

The conversion of compound 1 to compound 2 may be performed according to any method in the art. In one embodiment, compound 1 is treated with iso-propyl bromide and potassium carbonate in anhydrous DMF at a temperature above room temperature.

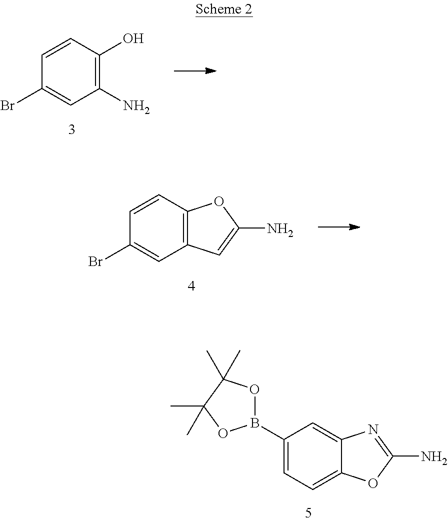 [SMILES: Nc1cc(Br)ccc1O ; Nc1cc2cc(Br)ccc2o1 ; CC1(C)OB(c2ccc3oc(N)nc3c2)OC1(C)C]

The conversion of compound 3 to compound 5 may be performed according to any method in the art. In one embodiment, compound 3 is treated with bromo cyanide in methanol to yield compound 4. Compound 4 may be converted to compound 5 via a transition-metal catalyzed cross-coupling reaction with a diboron reagent. In one embodiment, compound 4 is treated with bis(pinacolato)diboron, potassium acetate, and PdCl₂(dppf) in 1,4-dioxane at 110° C. to yield compound 5. In one embodiment, compound 5 is further treated with acid, for example 6N HCl, at elevated temperature, such as 80° C., to yield compound 5a, the boronic acid derivative.

Scheme 3

In one embodiment, the compound of Formula I is made by direct coupling according to the following scheme:

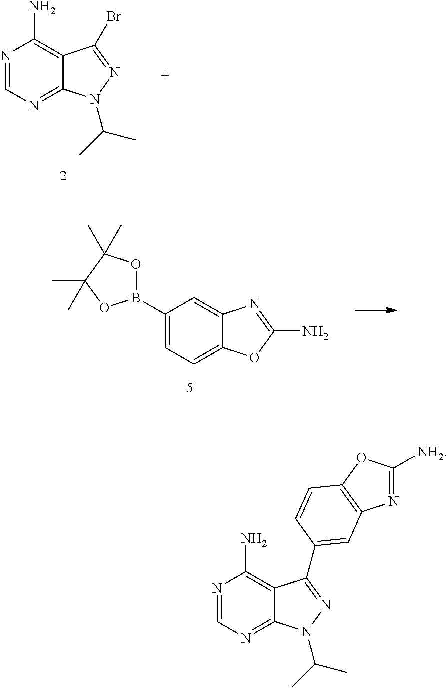 [SMILES: CC(C)n1nc(Br)c2c(N)ncnc21 ; CC1(C)OB(c2ccc3oc(N)nc3c2)OC1(C)C ; CC(C)n1nc(-c2ccc3oc(N)nc3c2)c2c(N)ncnc21]

The coupling of compound 2 with compound 5 may be performed under standard transition-metal catalyzed cross-coupling reaction conditions known in the art. In one embodiment, compounds 2 and 5 may be heated in a mixture of 1,4-dioxane and water in the presence of Pd(PPh₃)₄ and sodium carbonate at 110° C. to yield the compound of Formula I. Workup of the reaction product may include treatment of activated charcoal in MeOH, for example at reflux, to remove palladium.

Alternatively, the boronic acid derivative may be used according to the following scheme:

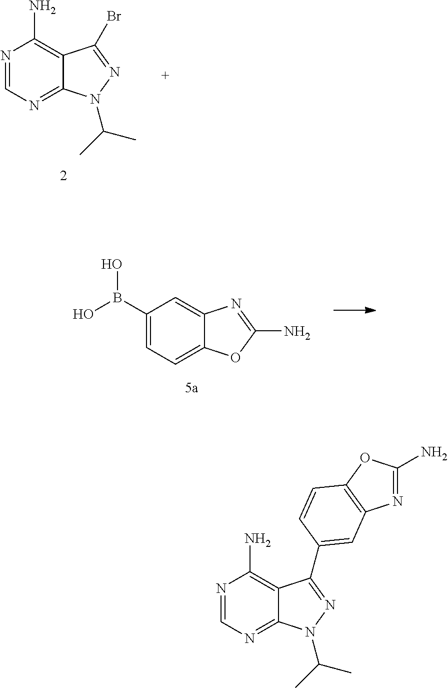 [SMILES: CC(C)n1nc(Br)c2c(N)ncnc21 ; Nc1nc2cc(B(O)O)ccc2o1 ; CC(C)n1nc(-c2ccc3oc(N)nc3c2)c2c(N)ncnc21]

The coupling of compound 2 with compound 5a may be performed under standard transition-metal catalyzed cross-coupling reaction conditions known in the art. Compound 5a can be a salt, such as the HCl salt, or internal salt, or a non-salt form. In one embodiment, compounds 2 and 5a may be heated in in a mixture of 1,4-dioxane and water in the presence of Pd(PPh₃)₄ and sodium carbonate at 110° C. to yield the compound of Formula I. Workup of the reaction product may include treatment of activated charcoal in MeOH, for example at reflux, to remove palladium.

The polymorphs according to the invention are not limited by the starting materials used to produce the compound of Formula I.

Isolation and purification of the chemical entities and intermediates described herein can be effected, if desired, by any suitable separation or purification procedure such as, for example, filtration, extraction, crystallization, column chromatography, thin-layer chromatography or thick-layer chromatography, or a combination of these procedures. Specific illustrations of suitable separation and isolation procedures can be had by reference to the examples below. However, other equivalent separation or isolation procedures can also be used. Prior to formulation as the active pharmaceutical ingredient in a drug product, the compound of Formula I may be isolated in greater than 90% purity, greater than 91% purity, greater than 92% purity, greater than 93% purity, greater than 94% purity, greater than 95% purity, greater than 96% purity, greater than 97% purity, greater than 98% purity, greater than 99% purity, and purity approaching 100%.

In one aspect, the invention is directed to methods of making polymorphs of the compound of the Formula I:

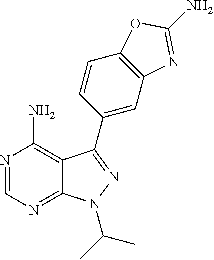 [SMILES: CC(C)n1nc(-c2ccc3oc(N)nc3c2)c2c(N)ncnc21]

or a pharmaceutically acceptable salt and/or solvate thereof either by isolation of the desired polymorph as the first solid form after synthesis of the compound of Formula I, or alternatively, by isolation of the desired polymorph as a transition from a prior solid form of the compound of Formula I. Transitions from one form to another are within the scope of the invention because they can be an alternative manufacturing method for obtaining the form desired for the production of the medicinal preparations.

In one embodiment, the desired polymorph is Form A, and the isolating step involves recrystallization of crude reaction product from a mono-solvent system. In various embodiments, the desired polymorph is Form A, and the isolating step involves recrystallization of crude product from a binary, tertiary, or greater solvent system, collectively understood as a multi-solvent system. In various embodiments, the desired polymorph is Form A, and the isolating step involves crystallization from a mono- or multi-solvent system, where the crystallization involves actively cooling the solution containing the dissolved compound of Formula I. In various embodiments, the desired polymorph is Form A, and the isolating step involves crystallization from a mono- or multi-solvent system, where the crystallization involves addition of an antisolvent either with or without an active cooling step to cause solid Form A to come out of solution.

In various embodiments, the desired polymorph is Form C, and the isolating step involves crystallization of crude reaction product from a mono-solvent system. In various embodiments, the desired polymorph is Form C, and the isolating step involves recrystallization of crude product from a binary, tertiary, or greater solvent system, where binary, tertiary, or greater solvent systems are collectively understood as multi-solvent systems. In various embodiments, the desired polymorph is Form C, and the isolating step involves crystallization from a mono- or multi-solvent system, where the crystallization involves actively cooling the solution containing the dissolved compound of Formula I. In various embodiments, the desired polymorph is Form C, and the isolating step involves crystallization from a mono- or multi-solvent system, where the crystallization involves addition of an antisolvent either with or without an active cooling step to cause solid Form C to come out of solution. In various embodiments, the conditions of crystallization are non-anhydrous. Where the conditions are non-anhydrous, water may be present in trace amounts, or in amounts less than 1% by volume of solvent. In various embodiments, water may be present as a co-solvent (or anti-solvent) in an amount between about 1% and about 50%. For example, water may be present in about 5%, about 10%, about 15%, about 20%, about 25%, about 30%, about 35%, about 40%, about 45%, and about 50% by volume of solvent. In various embodiments, water may be present in amounts equal to or greater than about 50% by volume of solvent. For example, water may be present in about 55%, about 60%, about 65%, about 70%, about 75%, about 80%, about 85%, about 90%, about 95%, and up to 100% by volume of solvent. In various embodiments, liquid water is present in a multi-solvent system in an amount between about 10% to about 50% by volume of the solvent system. In various embodiments, water may be present as water vapor or ambient humidity.

In various embodiments, the invention is directed to methods of making a polymorph of the compound of Formula I, wherein the method involves converting an isolated polymorph or mixture of polymorphs into a desired polymorph. In certain embodiments, the methods comprise exposing a composition comprising one or more polymorphs to conditions sufficient to convert at least about 50% of the total amount of original polymorph(s) into at least about 50% of the desired polymorph, and isolating the desired polymorph as needed.

In various embodiments, the original solid form of the compound of Formula I contains greater than about 50% non-Form C polymorphs, and the desired polymorph is Form C. The conversion to Form C may be performed under conditions with a multi-solvent system for a period of time sufficient to convert at least about 50% of the total amount of non-Form C polymorphs into Form C of the compound of Formula I, with an optional isolation of Form C from any non-Form C polymorphs, as needed. The multi-solvent system may include water. For example, conditions involving using the multi-solvent system may include dissolving the original composition in a water/organic solvent mixture at a temperature above 25° C. and then cool the resulting solution to 20° C. or lower.

In various embodiments, the original composition includes one or more of Form A, Form B, Form D, Form E, amorphous form, and mixtures thereof. In various embodiments, the original composition is greater than 50% by weight polymorph Form A.

In various embodiments, the invention is directed to compositions comprising a mixture of more than one polymorph of the compound of Formula I. For example, in various embodiments, the composition comprises a ratio of Form C to non-C polymorphs where the ratio is greater than 1:1, or greater than 9:1, or greater than 99:1. In various embodiments, the composition comprises both Form C and Form A.

Form A

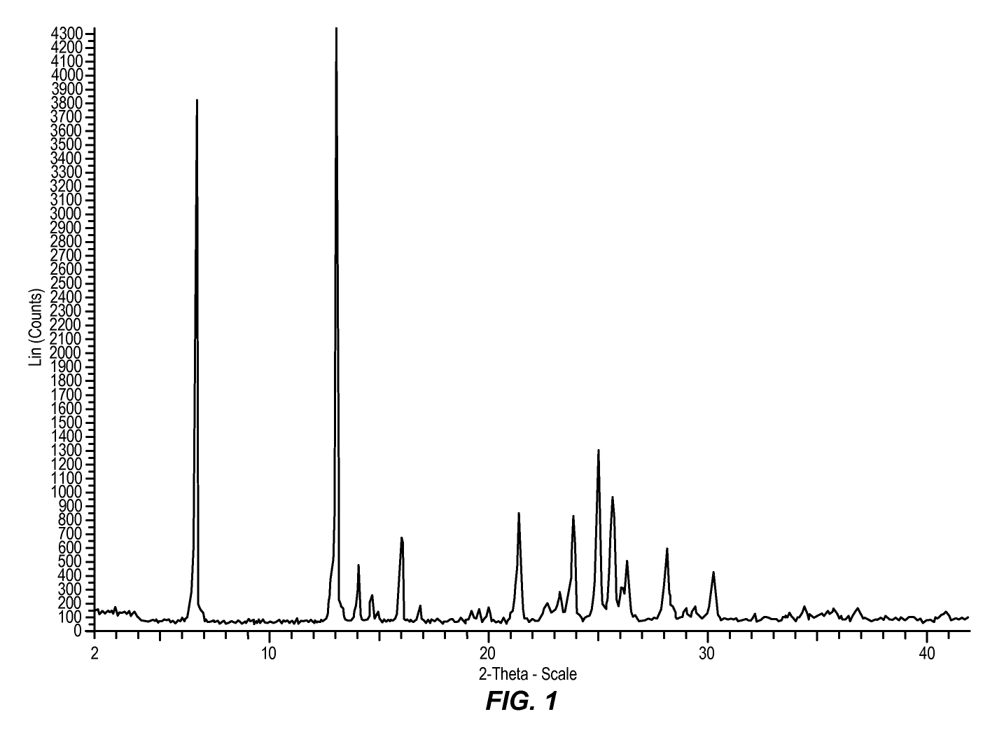

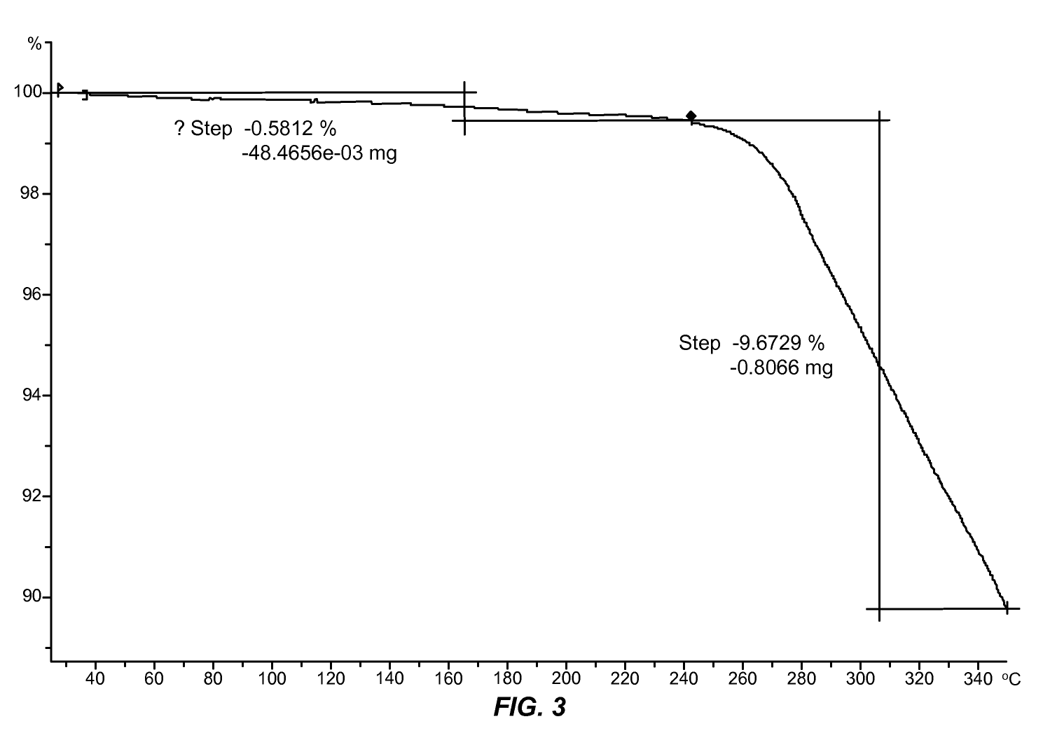

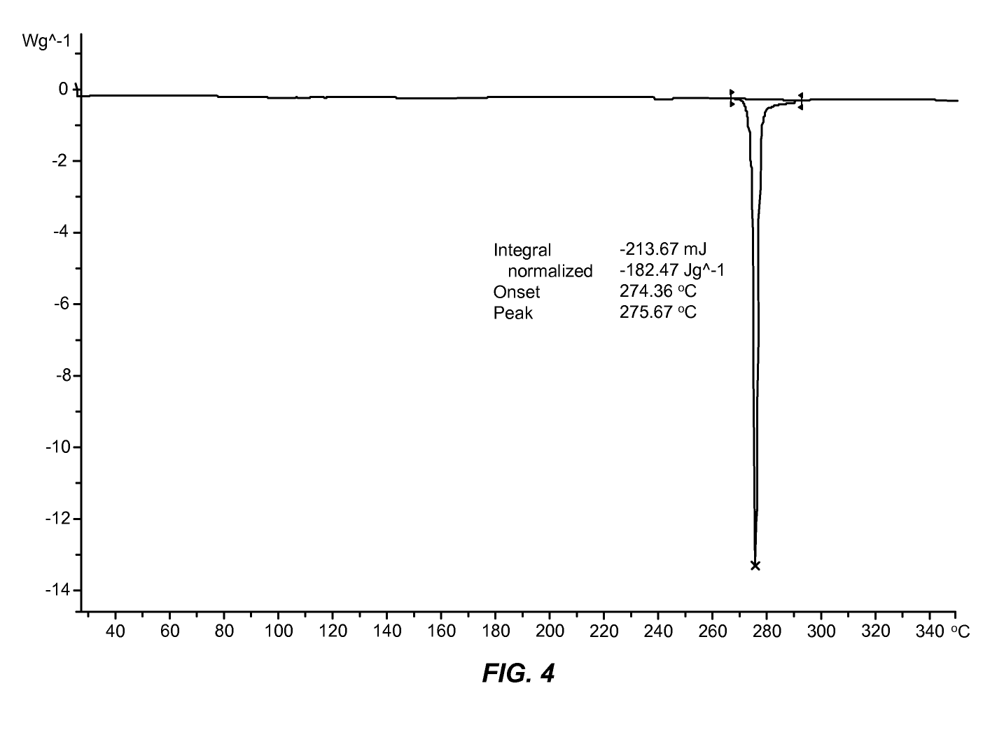

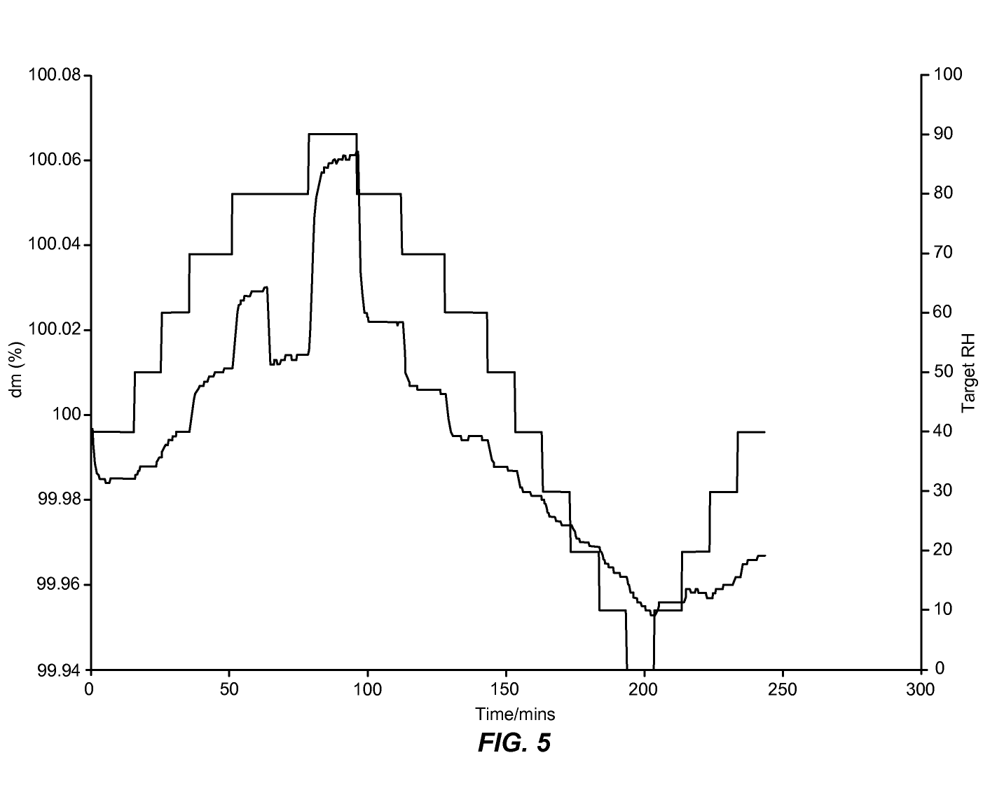

FIG. 1 shows the high-resolution XRPD diffractogram of polymorph Form A. FIG. 3 shows a thermogravimetric analysis (TGA) for Form A. FIG. 4 shows a differential scanning calorimetry (DSC) endotherm analysis for Form A. FIG. 5 shows a GVS kinetic plot for Form A.

In various embodiments, Form A may be obtained from direct workup of the synthetic step producing the compound of Formula I, and non-A Forms are not obtained, or are obtained as a minority component. In various embodiments, Form A may be obtained by fast and slow cooling crystallization from single solvent systems, including methanol and ethyl acetate. In various embodiments, Form A may be obtained by crystallization from a binary solvent system comprising ethyl acetate and methanol, as well as fast and slow cooling from binary solvent systems with dichloromethane or hexane as the anti-solvent. Form A may also be obtained from slurries in methanol, ethyl acetate, DMF, DMSO, N-methylpyrrolidone (NMP), acetic acid, isopropyl alcohol, acetonitrile, and dimethylacetamide (DMA). In various embodiments, Form A is obtained by re-slurrying one or more non-A Forms in an anhydrous solvent. For example, Form A is obtained by re-slurrying one or more non-A Forms (such as Form C) in methanol, chloroform, dichloromethane, isopropyl alcohol, ethanol, acetate, ethanol/acetate, or mixtures thereof.

Form C

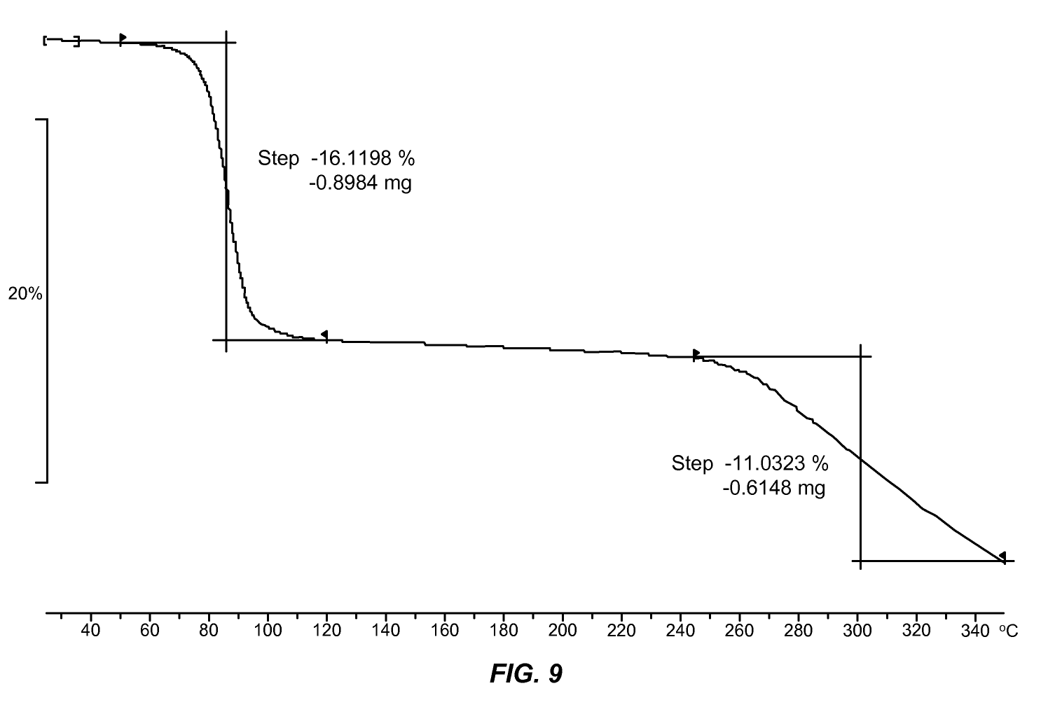

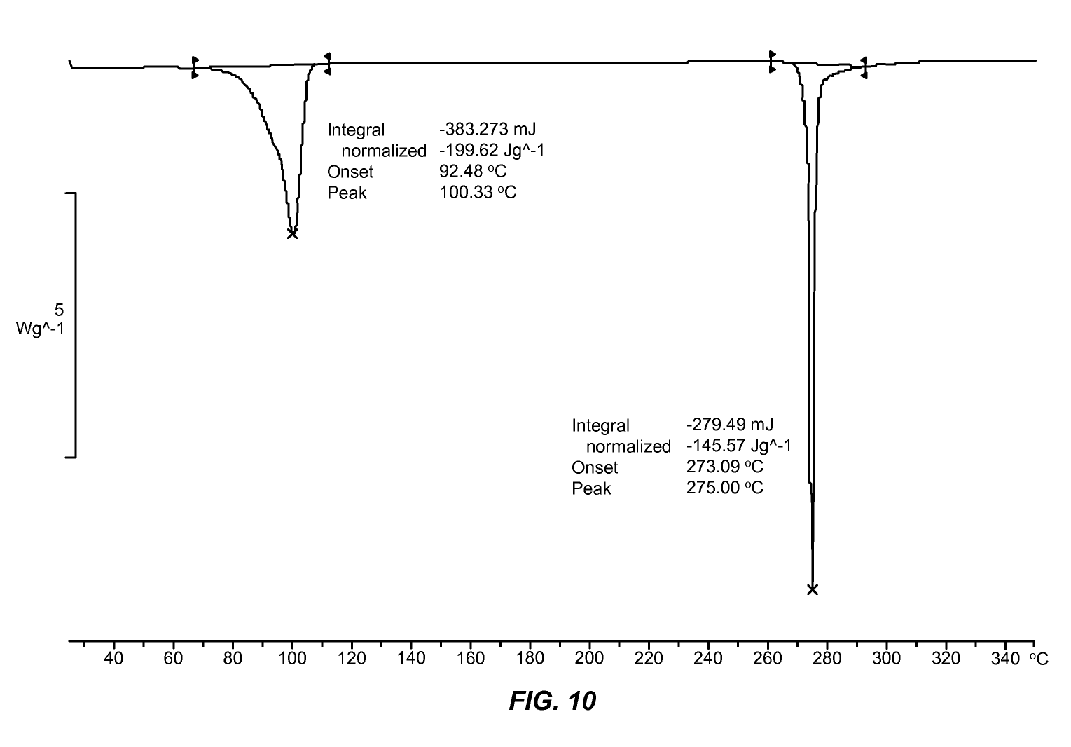

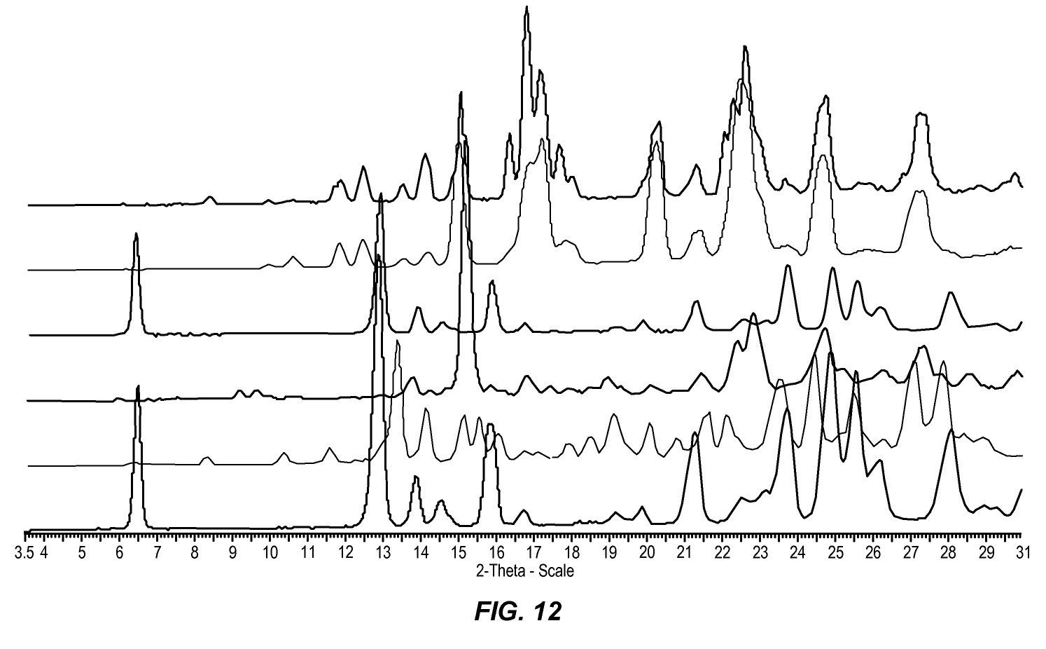

In one embodiment, the polymorph according to the invention is Form C. FIG. 12 shows the XRPD for Polymorph Form C. FIG. 9 shows a thermogravimetric analysis (TGA) for Form C. FIG. 10 shows a DSC endotherm analysis for Form C. The symbol “exo” indicates an exotherm. In some embodiments, Form C is characterized by a DSC trace showing a peak at about 100° C. and a peak at 275° C.

In various embodiments, Form C may be obtained in a mixture with non-C polymorph forms. For example, in various embodiments, Form C may be present as a composition further comprising one or more non-C polymorph forms. The amount of non-C polymorph forms may vary. For example, in various embodiments, the weight ratio of polymorph Form C to the total amount of one or more non-C polymorphs is greater than about 7:1, greater than about 8:1, greater than about 9:1, greater than about 9.5:1, or greater than about 99:1. Similarly, when formulated in pharmaceutical compositions, various amounts of non-C polymorph form may be present. In various embodiments the weight ratio of polymorph Form C to the total amount of one or more non-C polymorphs in a pharmaceutical composition may be greater than about 7:1, greater than about 8:1, greater than about 9:1, greater than about 9.5:1, or greater than about 99:1.

In various embodiments, Form C may be produced by placing Form A in water or a water-containing solvent system. Upon exposure to water or a water-containing solvent system, the combination may form a slurry. The combination of Form A and water or water-containing solvent system may be stirred, optionally with heating, until the desired amount of conversion of Form C has occurred. In various embodiments, the solvent system is a water-miscible alcohol with water. In various embodiments, the solvent system is a non-alcohol water-miscible solvent with water. In various embodiments, the solvent system is a common organic solvent, including THF or 1,4-dioxane with water. In various embodiments, Form C is produced by fast or slow cooling from binary solvent systems, including tetrahydrofuran or 1,4-dioxane as primary solvent, and water as anti-solvent.

Where a solvent in addition to water is used, the ratio of solvent to water may vary from about 100/1 to about 1/100. For example, the ratio of solvent to water may be selected from about 100/1, about 90/1, about 80/1, about 70/1, about 60/1, about 50/1, about 40/1, about 30/1, about 20/1, about 10/1, about 9/1, about 8/1, about 7/1, about 6/1, about 5/1, about 4/1, about 3/1, about 2/1, about 1.5/1, about 1/1, about 1/1.5, about 1/2, about 1/3, about 1/4, about 1/5, about 1/6, about 1/7, about 1/8, about 1/9, about 1/10, about 1/20, about 1/30, about 1/40, about 1/50, about 1/60, about 1/70, about 1/80, about 1/90, and about 1/100. The total amount of solvent or solvent system may be selected from about 0.1 volumes (e.g. liters/kg), about 0.5 volumes, about 1 volume, about 2 volumes, about 3 volumes, 4 about volumes, about 5 volumes, about 6 volumes, about 7 volumes, about 8 volumes, about 9 volumes, about 10 volumes, about 11 volumes, about 12 volumes, about 13 volumes, about 14 volumes, about 15 volumes, about 16 volumes, about 17 volumes, about 18 volumes, about 19 volumes, about 20 volumes, about 30 volumes, about 40 volumes, about 50 volumes, or more. In various embodiments, the solvent system is THF/water. In various embodiments, the solvent system is dioxane/water.

In various embodiments, Form C is obtained by recrystallization of a non-C Form, including complete dissolution of the non-C Form followed by filtration to remove any insoluble particles, and subsequent crystallization to yield Form C. In various embodiments, complete dissolution and filtration is not performed, in which case a slurry is formed which converts to Form C without complete dissolution of one or more non-C Forms. In various embodiments, Form C is a channel hydrate.

In various embodiments, a method is disclosed of making polymorph Form A of the compound of Formula I:

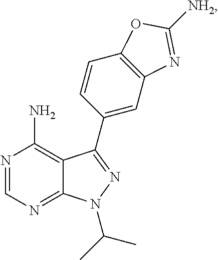 [SMILES: CC(C)n1nc(-c2ccc3oc(N)nc3c2)c2c(N)ncnc21]

said method comprising

(i) reacting compounds 2 and 5:

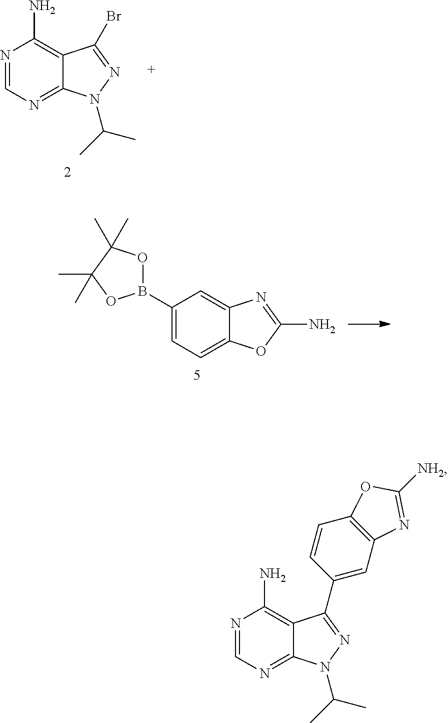 [SMILES: CC(C)n1nc(Br)c2c(N)ncnc21 ; CC1(C)OB(c2ccc3oc(N)nc3c2)OC1(C)C ; CC(C)n1nc(-c2ccc3oc(N)nc3c2)c2c(N)ncnc21]

or reacting compounds 2 and 5a:

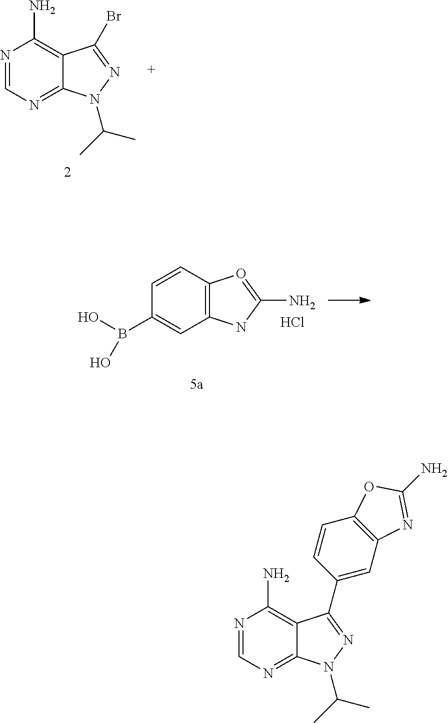

to yield a compound of Formula I; and (ii) isolating said compound of Formula I in polymorph Form A, wherein said isolation occurs under conditions to remove palladium. For example, palladium is removed by treatment of the compound of Formula I with activated charcoal. In various embodiments, the treatment of the compound of Formula I includes methanol at reflux. Upon treatment to remove palladium, the isolated polymorph Form A contains an amount of palladium selected from less than about 1% by weight, less than about 0.5% by weight, less than about 0.1% by weight, less than about 0.05% by weight, less than about 0.01% by weight, less than about 0.001% by weight, and less than about 0.0001% by weight.

Salt Forms

In various embodiments, the compound of Formula I is a pharmaceutically acceptable salt. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, benzene sulfonic acid, salicylic acid, 1,2-ethane disulfonic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. In some embodiments, the pharmaceutically acceptable base addition salt is chosen from ammonium, potassium, sodium, calcium, and magnesium salts. Bis salts (i.e. two counterions) and higher salts are encompassed within the meaning of pharmaceutically acceptable salts.

In various embodiments, salts of Formula I may be formed with sulfuric acid, p-toluenesulfonic acid, D-glucaronic acid, ethane-1,2-disulfonic acid (EDSA), 2-naphthalenesulfonic acid (NSA), hydrochloric acid (HCl) (mono and bis), hydrobromic acid (HBr), oxalic acid, naphthalene-1,5-disulfonic acid (NDSA), DL-mandelic acid, fumaric acid, sulfuric acid, maleic acid, methanesulfonic acid (MSA), benzenesulfonic acid (BSA), ethanesulfonic acid (ESA), L-malic acid, phosphoric acid, and aminoethanesulfonic acid (taurine).

Deuterium Atoms

In various embodiments, the compound of Formula I is modified by replacing one or more hydrogen atoms with deuterium atoms. In various embodiments, the one or more hydrogen atoms to be replaced are selected from hydrogens attached to a carbon atom, for example, as represented by H1-H11 in the following formula:

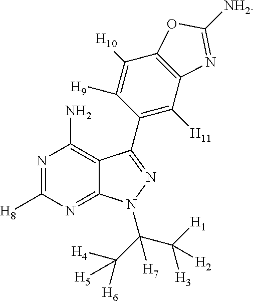

The hydrogen(s) to be replaced may be selected from one or more of H1-H7 as shown in the above formula. The number of deuterium atoms to be incorporated in the compound of Formula I may range from one deuterium atom to replacement of all hydrogen atoms with deuterium atoms. For example, in various embodiments, one to six hydrogens designated as H1-H6 in the above formula are replaced with one to six deuterium atoms in any combination. As such, the compound of Formula I may have two CD₃ groups, one CD₃ group and a CHD₂ group, one CD₃ group and a CH₂D group, one CD₃ group and a CH₃ group, two CHD₂ groups, one CHD₂ group and a CH₂D group, one CHD₂ group and a CH₃ group, two CH₂D groups, one CH₂D group and a CH₃ group, and the like.

In various embodiments, deuterium-labeled compounds of Formula I exhibit improved metabolic stability compared to non-labeled compounds. Alternatively, deuterium-labeled compounds are useful in analysis of the compound, such as in NMR, or in analysis of metabolic pathways.

Formulas II and III

In various embodiments, the compounds of Formulas II and III are synthesized:

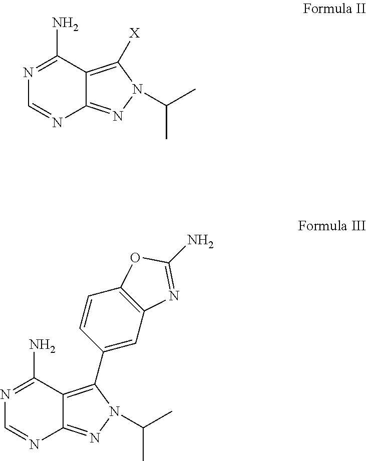

where X in Formula II is a halogen. In various embodiments, X is selected from iodine and bromine

In various embodiments, the synthesis of the compound of Formula I also results in compounds of Formula II and/or III. For example, in performing the synthesis disclosed herein in Schemes 1 and 3, compounds according to Formula II and III may be synthesized. In various embodiments, compounds according to Formula II or III are removed or separated from the compound of Formula I or its precursors. For example, in Scheme 1 above, where Compound 2 is in combination with the compound of Formula II, Compound 2 is purified to reduce the amount of compound of Formula II to less than 50% by weight, less than 40% by weight, less than 30% by weight, less than 20% by weight, less than 10% by weight, less than 5% by weight, less than 4% by weight, less than 3% by weight, less than 2% by weight, less than 1% by weight, less than 0.1% by weight, or less than 0.01% by weight, all amounts being about. With respect to the compound of Formula I, where the compound of Formula I is in combination with the compound of Formula III, the compound of Formula I is purified to reduce the amount of compound of Formula III to less than 50% by weight, less than 40% by weight, less than 30% by weight, less than 20% by weight, less than 10% by weight, less than 5% by weight, less than 4% by weight, less than 3% by weight, less than 2% by weight, less than 1% by weight, less than 0.1% by weight, or less than 0.01% by weight, all amounts being about.

### III. Compositions

The invention provides compositions, including pharmaceutical compositions, comprising one or more polymorphs of the present invention.

In various embodiments, the ratio of desired polymorph such as Form A or Form C to all other polymorphs may be greater than about 5:1, 6:1, 7:1, 8:1, 9:1, or more.

The subject pharmaceutical compositions are typically formulated to provide a therapeutically effective amount of a polymorph of the present invention as the active ingredient, or a pharmaceutically acceptable salt, ester, prodrug, solvate, hydrate or derivative thereof. Where desired, the pharmaceutical compositions contain pharmaceutically acceptable salt and/or coordination complex thereof, and one or more pharmaceutically acceptable excipients, carriers, including inert solid diluents and fillers, diluents, including sterile aqueous solution and various organic solvents, permeation enhancers, solubilizers and adjuvants.

The subject pharmaceutical compositions can be administered alone or in combination with one or more other agents, which are also typically administered in the form of pharmaceutical compositions. Where desired, the subject polymorphs and other agent(s) may be mixed into a preparation or both components may be formulated into separate preparations to use them in combination separately or at the same time.

In some embodiments, the concentration of one or more of the polymorphs provided in the pharmaceutical compositions of the present invention is less than 100%, 90%, 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, 2%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% w/w, w/v or v/v.

In some embodiments, the concentration of one or more of the polymorphs of the present invention is greater than 90%, 80%, 70%, 60%, 50%, 40%, 30%, 20%, 19.75%, 19.50%, 19.25% 19%, 18.75%, 18.50%, 18.25% 18%, 17.75%, 17.50%, 17.25% 17%, 16.75%, 16.50%, 16.25% 16%, 15.75%, 15.50%, 15.25% 15%, 14.75%, 14.50%, 14.25% 14%, 13.75%, 13.50%, 13.25% 13%, 12.75%, 12.50%, 12.25% 12%, 11.75%, 11.50%, 11.25% 11%, 10.75%, 10.50%, 10.25% 10%, 9.75%, 9.50%, 9.25% 9%, 8.75%, 8.50%, 8.25% 8%, 7.75%, 7.50%, 7.25% 7%, 6.75%, 6.50%, 6.25% 6%, 5.75%, 5.50%, 5.25% 5%, 4.75%, 4.50%, 4.25%, 4%, 3.75%, 3.50%, 3.25%, 3%, 2.75%, 2.50%, 2.25%, 2%, 1.75%, 1.50%, 125%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% w/w, w/v, or v/v.

In some embodiments, the concentration of one or more of the polymorphs of the present invention is in the range from approximately 0.0001% to approximately 50%, approximately 0.001% to approximately 40%, approximately 0.01% to approximately 30%, approximately 0.02% to approximately 29%, approximately 0.03% to approximately 28%, approximately 0.04% to approximately 27%, approximately 0.05% to approximately 26%, approximately 0.06% to approximately 25%, approximately 0.07% to approximately 24%, approximately 0.08% to approximately 23%, approximately 0.09% to approximately 22%, approximately 0.1% to approximately 21%, approximately 0.2% to approximately 20%, approximately 0.3% to approximately 19%, approximately 0.4% to approximately 18%, approximately 0.5% to approximately 17%, approximately 0.6% to approximately 16%, approximately 0.7% to approximately 15%, approximately 0.8% to approximately 14%, approximately 0.9% to approximately 12%, approximately 1% to approximately 10% w/w, w/v or v/v. v/v.

In some embodiments, the concentration of one or more of the polymorphs of the present invention is in the range from approximately 0.001% to approximately 10%, approximately 0.01% to approximately 5%, approximately 0.02% to approximately 4.5%, approximately 0.03% to approximately 4%, approximately 0.04% to approximately 3.5%, approximately 0.05% to approximately 3%, approximately 0.06% to approximately 2.5%, approximately 0.07% to approximately 2%, approximately 0.08% to approximately 1.5%, approximately 0.09% to approximately 1%, approximately 0.1% to approximately 0.9% w/w, w/v or v/v.

In some embodiments, the amount of one or more of the polymorphs of the present invention is equal to or less than 10 g, 9.5 g, 9.0 g, 8.5 g, 8.0 g, 7.5 g, 7.0 g, 6.5 g, 6.0 g, 5.5 g, 5.0 g, 4.5 g, 4.0 g, 3.5 g, 3.0 g, 2.5 g, 2.0 g, 1.5 g, 1.0 g, 0.95 g, 0.9 g, 0.85 g, 0.8 g, 0.75 g, 0.7 g, 0.65 g, 0.6 g, 0.55 g, 0.5 g, 0.45 g, 0.4 g, 0.35 g, 0.3 g, 0.25 g, 0.2 g, 0.15 g, 0.1 g, 0.09 g, 0.08 g, 0.07 g, 0.06 g, 0.05 g, 0.04 g, 0.03 g, 0.02 g, 0.01 g, 0.009 g, 0.008 g, 0.007 g, 0.006 g, 0.005 g, 0.004 g, 0.003 g, 0.002 g, 0.001 g, 0.0009 g, 0.0008 g, 0.0007 g, 0.0006 g, 0.0005 g, 0.0004 g, 0.0003 g, 0.0002 g, or 0.0001 g.

In some embodiments, the amount of one or more of the polymorphs of the present invention is more than 0.0001 g, 0.0002 g, 0.0003 g, 0.0004 g, 0.0005 g, 0.0006 g, 0.0007 g, 0.0008 g, 0.0009 g, 0.001 g, 0.0015 g, 0.002 g, 0.0025 g, 0.003 g, 0.0035 g, 0.004 g, 0.0045 g, 0.005 g, 0.0055 g, 0.006 g, 0.0065 g, 0.007 g, 0.0075 g, 0.008 g, 0.0085 g, 0.009 g, 0.0095 g, 0.01 g, 0.015 g, 0.02 g, 0.025 g, 0.03 g, 0.035 g, 0.04 g, 0.045 g, 0.05 g, 0.055 g, 0.06 g, 0.065 g, 0.07 g, 0.075 g, 0.08 g, 0.085 g, 0.09 g, 0.095 g, 0.1 g, 0.15 g, 0.2 g, 0.25 g, 0.3 g, 0.35 g, 0.4 g, 0.45 g, 0.5 g, 0.55 g, 0.6 g, 0.65 g, 0.7 g, 0.75 g, 0.8 g, 0.85 g, 0.9 g, 0.95 g, 1 g, 1.5 g, 2 g, 2.5, 3 g, 3.5, 4 g, 4.5 g, 5 g, 5.5 g, 6 g, 6.5 g, 7 g, 7.5 g, 8 g, 8.5 g, 9 g, 9.5 g, or 10 g.

In some embodiments, the amount of one or more of the polymorphs of the present invention is in the range of 0.0001-10 g, 0.0005-9 g, 0.001-8 g, 0.005-7 g, 0.01-6 g, 0.05-5 g, 0.1-4 g, 0.5-4 g, or 1-3 g.

The polymorphs according to the invention are effective over a wide dosage range. For example, in the treatment of adult humans, dosages from 0.01 to 1000 mg, from 0.5 to 100 mg, from 1 to 50 mg per day, and from 5 to 40 mg per day are examples of dosages that may be used. An exemplary dosage is 10 to 30 mg per day or per week. The exact dosage will depend upon the route of administration, the form in which the polymorphs is administered, the subject to be treated, the body weight of the subject to be treated, and the preference and experience of the attending physician.

In various embodiments, pharmaceutical compositions of Formula I also contain an amount of the compound of Formula III. For example, a composition of Formula I may comprise a detectable amount of the compound of Formula III. In various embodiments, the amount of compound of Formula III is less than 50% by weight, less than 40% by weight, less than 30% by weight, less than 20% by weight, less than 10% by weight, less than 5% by weight, less than 4% by weight, less than 3% by weight, less than 2% by weight, less than 1% by weight, less than 0.1% by weight, or less than 0.01% by weight, all amounts being about, with respect to the amount of Formula I.

Described below are non-limiting exemplary pharmaceutical compositions and methods for preparing the same.

Pharmaceutical compositions for oral administration: In some embodiments, the invention provides a pharmaceutical composition for oral administration containing a polymorph of the present invention, and a pharmaceutical excipient suitable for oral administration.

In some embodiments, the invention provides a solid pharmaceutical composition for oral administration containing: (i) an effective amount of a compound of the present invention; optionally (ii) an effective amount of a second agent; and (iii) one or more pharmaceutical excipients suitable for oral administration. In some embodiments, the composition further contains: (iv) an effective amount of a third agent.

In some embodiments, the pharmaceutical composition may be a liquid pharmaceutical composition suitable for oral consumption. Pharmaceutical compositions of the invention suitable for oral administration can be presented as discrete dosage forms, such as capsules, cachets, or tablets, or liquids or aerosol sprays each containing a predetermined amount of an active ingredient as a powder or in granules, a solution, or a suspension in an aqueous or non-aqueous liquid, an oil-in-water emulsion, or a water-in-oil liquid emulsion. Such dosage forms can be prepared by any of the methods of pharmacy, but all methods include the step of bringing the active ingredient into association with the carrier, which constitutes one or more necessary ingredients. In general, the compositions are prepared by uniformly and intimately admixing the active ingredient with liquid carriers or finely divided solid carriers or both, and then, if necessary, shaping the product into the desired presentation. For example, a tablet can be prepared by compression or molding, optionally with one or more accessory ingredients. Compressed tablets can be prepared by compressing in a suitable machine the active ingredient in a free-flowing form such as powder or granules, optionally mixed with an excipient such as, but not limited to, a binder, a lubricant, an inert diluent, and/or a surface active or dispersing agent. Molded tablets can be made by molding in a suitable machine a mixture of the powdered compound moistened with an inert liquid diluent.

This invention further encompasses anhydrous pharmaceutical compositions and dosage forms comprising an active ingredient, since water can facilitate the degradation of some compounds. For example, water may be added (e.g., 5%) in the pharmaceutical arts as a means of simulating long-term storage in order to determine characteristics such as shelf-life or the stability of formulations over time. Anhydrous pharmaceutical compositions and dosage forms of the invention can be prepared using anhydrous or low moisture containing ingredients and low moisture or low humidity conditions. Pharmaceutical compositions and dosage forms of the invention which contain lactose can be made anhydrous if substantial contact with moisture and/or humidity during manufacturing, packaging, and/or storage is expected. An anhydrous pharmaceutical composition may be prepared and stored such that its anhydrous nature is maintained. Accordingly, anhydrous compositions may be packaged using materials known to prevent exposure to water such that they can be included in suitable formulary kits. Examples of suitable packaging include, but are not limited to, hermetically sealed foils, plastic or the like, unit dose containers, blister packs, and strip packs.

An active ingredient can be combined in an intimate admixture with a pharmaceutical carrier according to conventional pharmaceutical compounding techniques. The carrier can take a wide variety of forms depending on the form of preparation desired for administration. In preparing the compositions for an oral dosage form, any of the usual pharmaceutical media can be employed as carriers, such as, for example, water, glycols, oils, alcohols, flavoring agents, preservatives, coloring agents, and the like in the case of oral liquid preparations (such as suspensions, solutions, and elixirs) or aerosols; or carriers such as starches, sugars, microcrystalline cellulose, diluents, granulating agents, lubricants, binders, and disintegrating agents can be used in the case of oral solid preparations, in some embodiments without employing the use of lactose. For example, suitable carriers include powders, capsules, and tablets, with the solid oral preparations. If desired, tablets can be coated by standard aqueous or nonaqueous techniques.

Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, microcrystalline cellulose, and mixtures thereof.

Examples of suitable fillers for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof.

Disintegrants may be used in the compositions of the invention to provide tablets that disintegrate when exposed to an aqueous environment. Too much of a disintegrant may produce tablets which may disintegrate in the bottle. Too little may be insufficient for disintegration to occur and may thus alter the rate and extent of release of the active ingredient(s) from the dosage form. Thus, a sufficient amount of disintegrant that is neither too little nor too much to detrimentally alter the release of the active ingredient(s) may be used to form the dosage forms of the polymorphs disclosed herein. The amount of disintegrant used may vary based upon the type of formulation and mode of administration, and may be readily discernible to those of ordinary skill in the art. About 0.5 to about 15 weight percent of disintegrant, or about 1 to about 5 weight percent of disintegrant, may be used in the pharmaceutical composition. Disintegrants that can be used to form pharmaceutical compositions and dosage forms of the invention include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums or mixtures thereof.

Lubricants which can be used to form pharmaceutical compositions and dosage forms of the invention include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethylaureate, agar, or mixtures thereof. Additional lubricants include, for example, a syloid silica gel, a coagulated aerosol of synthetic silica, or mixtures thereof. A lubricant can optionally be added, in an amount of less than about 1 weight percent of the pharmaceutical composition.

In some cases, colloid particles include at least one cationic agent and at least one non-ionic surfactant such as a poloxamer, tyloxapol, a polysorbate, a polyoxyethylene castor oil derivative, a sorbitan ester, or a polyoxyl stearate. In some cases, the cationic agent is an alkylamine, a tertiary alkyl amine, a quaternary ammonium compound, a cationic lipid, an amino alcohol, a biguanidine salt, a cationic compound or a mixture thereof. In some cases the cationic agent is a biguanidine salt such as chlorhexidine, polyaminopropyl biguanidine, phenformin, alkylbiguanidine, or a mixture thereof. In some cases, the quaternary ammonium compound is a benzalkonium halide, lauralkonium halide, cetrimide, hexadecyltrimethylammonium halide, tetradecyltrimethylammonium halide, dodecyltrimethylammonium halide, cetrimonium halide, benzethonium halide, behenalkonium halide, cetalkonium halide, cetethyldimonium halide, cetylpyridinium halide, benzododecinium halide, chlorallyl methenamine halide, rnyristylalkonium halide, stearalkonium halide or a mixture of two or more thereof. In some cases, cationic agent is a benzalkonium chloride, lauralkonium chloride, benzododecinium bromide, benzethenium chloride, hexadecyltrimethylammonium bromide, tetradecyltrimethylammonium bromide, dodecyltrimethylammonium bromide or a mixture of two or more thereof. In some cases, the oil phase is mineral oil and light mineral oil, medium chain triglycerides (MCT), coconut oil; hydrogenated oils comprising hydrogenated cottonseed oil, hydrogenated palm oil, hydrogenate castor oil or hydrogenated soybean oil; polyoxyethylene hydrogenated castor oil derivatives comprising polyoxyl-40 hydrogenated castor oil, polyoxyl-60 hydrogenated castor oil or polyoxyl-100 hydrogenated castor oil.

When aqueous suspensions and/or elixirs are desired for oral administration, the active ingredient therein may be combined with various sweetening or flavoring agents, coloring matter or dyes and, if so desired, emulsifying and/or suspending agents, together with such diluents as water, ethanol, propylene glycol, glycerin and various combinations thereof.

The tablets can be uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

Surfactant which can be used to form pharmaceutical compositions and dosage forms of the invention include, but are not limited to, hydrophilic surfactants, lipophilic surfactants, and mixtures thereof. That is, a mixture of hydrophilic surfactants may be employed, a mixture of lipophilic surfactants may be employed, or a mixture of at least one hydrophilic surfactant and at least one lipophilic surfactant may be employed.

A suitable hydrophilic surfactant may generally have an HLB value of at least 10, while suitable lipophilic surfactants may generally have an HLB value of or less than about 10. An empirical parameter used to characterize the relative hydrophilicity and hydrophobicity of non-ionic amphiphilic compounds is the hydrophilic-lipophilic balance (“HLB” value). Surfactants with lower HLB values are more lipophilic or hydrophobic, and have greater solubility in oils, while surfactants with higher HLB values are more hydrophilic, and have greater solubility in aqueous solutions. Hydrophilic surfactants are generally considered to be those compounds having an HLB value greater than about 10, as well as anionic, cationic, or zwitterionic compounds for which the HLB scale is not generally applicable. Similarly, lipophilic (i.e., hydrophobic) surfactants are compounds having an HLB value equal to or less than about 10. However, HLB value of a surfactant is merely a rough guide generally used to enable formulation of industrial, pharmaceutical and cosmetic emulsions.

Hydrophilic surfactants may be either ionic or non-ionic. Suitable ionic surfactants include, but are not limited to, alkylammonium salts; fusidic acid salts; fatty acid derivatives of amino acids, oligopeptides, and polypeptides; glyceride derivatives of amino acids, oligopeptides, and polypeptides; lecithins and hydrogenated lecithins; lysolecithins and hydrogenated lysolecithins; phospholipids and derivatives thereof; lysophospholipids and derivatives thereof; carnitine fatty acid ester salts; salts of alkylsulfates; fatty acid salts; sodium docusate; acylactylates; mono- and di-acetylated tartaric acid esters of mono- and di-glycerides; succinylated mono- and di-glycerides; citric acid esters of mono- and di-glycerides; and mixtures thereof.

Within the aforementioned group, ionic surfactants include, by way of example: lecithins, lysolecithin, phospholipids, lysophospholipids and derivatives thereof; carnitine fatty acid ester salts; salts of alkylsulfates; fatty acid salts; sodium docusate; acylactylates; mono- and di-acetylated tartaric acid esters of mono- and di-glycerides; succinylated mono- and di-glycerides; citric acid esters of mono- and di-glycerides; and mixtures thereof.

Ionic surfactants may be the ionized forms of lecithin, lysolecithin, phosphatidylcholine, phosphatidylethanolamine, phosphatidylglycerol, phosphatidic acid, phosphatidylserine, lysophosphatidylcholine, lysophosphatidylethanolamine, lysophosphatidylglycerol, lysophosphatidic acid, lysophosphatidylserine, PEG-phosphatidylethanolamine, PVP-phosphatidylethanolamine, lactylic esters of fatty acids, stearoyl-2-lactylate, stearoyl lactylate, succinylated monoglycerides, mono/diacetylated tartaric acid esters of mono/diglycerides, citric acid esters of mono/diglycerides, cholylsarcosine, caproate, caprylate, caprate, laurate, myristate, palmitate, oleate, ricinoleate, linoleate, linolenate, stearate, lauryl sulfate, teracecyl sulfate, docusate, lauroyl carnitines, palmitoyl carnitines, myristoyl carnitines, and salts and mixtures thereof.

Hydrophilic non-ionic surfactants may include, but are not limited to, alkylglucosides; alkylmaltosides; alkylthioglucosides; lauryl macrogolglycerides; polyoxyalkylene alkyl ethers such as polyethylene glycol alkyl ethers; polyoxyalkylene alkylphenols such as polyethylene glycol alkyl phenols; polyoxyalkylene alkyl phenol fatty acid esters such as polyethylene glycol fatty acids monoesters and polyethylene glycol fatty acids diesters; polyethylene glycol glycerol fatty acid esters; polyglycerol fatty acid esters; polyoxyalkylene sorbitan fatty acid esters such as polyethylene glycol sorbitan fatty acid esters; hydrophilic transesterification products of a polyol with at least one member of the group consisting of glycerides, vegetable oils, hydrogenated vegetable oils, fatty acids, and sterols; polyoxyethylene sterols, derivatives, and analogues thereof; polyoxyethylated vitamins and derivatives thereof; polyoxyethylene-polyoxypropylene block copolymers; and mixtures thereof; polyethylene glycol sorbitan fatty acid esters and hydrophilic transesterification products of a polyol with at least one member of the group consisting of triglycerides, vegetable oils, and hydrogenated vegetable oils. The polyol may be glycerol, ethylene glycol, polyethylene glycol, sorbitol, propylene glycol, pentaerythritol, or a saccharide.

Other hydrophilic-non-ionic surfactants include, without limitation, PEG-10 laurate, PEG-12 laurate, PEG-20 laurate, PEG-32 laurate, PEG-32 dilaurate, PEG-12 oleate, PEG-15 oleate, PEG-20 oleate, PEG-20 dioleate, PEG-32 oleate, PEG-200 oleate, PEG-400 oleate, PEG-15 stearate, PEG-32 distearate, PEG-40 stearate, PEG-100 stearate, PEG-20 dilaurate, PEG-25 glyceryl trioleate, PEG-32 dioleate, PEG-20 glyceryl laurate, PEG-30 glyceryl laurate, PEG-20 glyceryl stearate, PEG-20 glyceryl oleate, PEG-30 glyceryl oleate, PEG-30 glyceryl laurate, PEG-40 glyceryl laurate, PEG-40 palm kernel oil, PEG-50 hydrogenated castor oil, PEG-40 castor oil, PEG-35 castor oil, PEG-60 castor oil, PEG-40 hydrogenated castor oil, PEG-60 hydrogenated castor oil, PEG-60 corn oil, PEG-6 caprate/caprylate glycerides, PEG-8 caprate/caprylate glycerides, polyglyceryl-10 laurate, PEG-30 cholesterol, PEG-25 phyto sterol, PEG-30 soya sterol, PEG-20 trioleate, PEG-40 sorbitan oleate, PEG-80 sorbitan laurate, polysorbate 20, polysorbate 80, POE-9 lauryl ether, POE-23 lauryl ether, POE-10 oleyl ether, POE-20 oleyl ether, POE-20 stearyl ether, tocopheryl PEG-100 succinate, PEG-24 cholesterol, polyglyceryl-10oleate, Tween 40, Tween 60, sucrose monostearate, sucrose monolaurate, sucrose monopalmitate, PEG 10-100 nonyl phenol series, PEG 15-100 octyl phenol series, and poloxamers.

Suitable lipophilic surfactants include, by way of example only: fatty alcohols; glycerol fatty acid esters; acetylated glycerol fatty acid esters; lower alcohol fatty acids esters; propylene glycol fatty acid esters; sorbitan fatty acid esters; polyethylene glycol sorbitan fatty acid esters; sterols and sterol derivatives; polyoxyethylated sterols and sterol derivatives; polyethylene glycol alkyl ethers; sugar esters; sugar ethers; lactic acid derivatives of mono- and di-glycerides; hydrophobic transesterification products of a polyol with at least one member of the group consisting of glycerides, vegetable oils, hydrogenated vegetable oils, fatty acids and sterols; oil-soluble vitamins/vitamin derivatives; and mixtures thereof. Within this group, preferred lipophilic surfactants include glycerol fatty acid esters, propylene glycol fatty acid esters, and mixtures thereof, or are hydrophobic transesterification products of a polyol with at least one member of the group consisting of vegetable oils, hydrogenated vegetable oils, and triglycerides.

In one embodiment, the composition may include a solubilizer to ensure good solubilization and/or dissolution of the compound of the present invention and to minimize precipitation of the compound of the present invention. This can be especially important for compositions for non-oral use, e.g., compositions for injection. A solubilizer may also be added to increase the solubility of the hydrophilic drug and/or other components, such as surfactants, or to maintain the composition as a stable or homogeneous solution or dispersion.

Examples of suitable solubilizers include, but are not limited to, the following: alcohols and polyols, such as ethanol, isopropyl alcohol, butanol, benzyl alcohol, ethylene glycol, propylene glycol, butanediols and isomers thereof, glycerol, pentaerythritol, sorbitol, mannitol, transcutol, dimethyl isosorbide, polyethylene glycol, polypropylene glycol, polyvinylalcohol, hydroxypropyl methylcellulose and other cellulose derivatives, cyclodextrins and cyclodextrin derivatives; ethers of polyethylene glycols having an average molecular weight of about 200 to about 6000, such as tetrahydrofurfuryl alcohol PEG ether (glycofurol) or methoxy PEG; amides and other nitrogen-containing compounds such as 2-pyrrolidone, 2-piperidone, 8-caprolactam, N-alkylpyrrolidone, N-hydroxyalkylpyrrolidone, N-alkylpiperidone, N-alkylcaprolactam, dimethylacetamide and polyvinylpyrrolidone; esters such as ethyl propionate, tributylcitrate, acetyl triethylcitrate, acetyl tributyl citrate, triethylcitrate, ethyl oleate, ethyl caprylate, ethyl butyrate, triacetin, propylene glycol monoacetate, propylene glycol diacetate, ε-caprolactone and isomers thereof, δ-valerolactone and isomers thereof, β-butyrolactone and isomers thereof; and other solubilizers known in the art, such as dimethyl acetamide, dimethyl isosorbide, N-methyl pyrrolidones, monooctanoin, diethylene glycol monoethyl ether, and water. In various embodiments, a solubilizer comprising polyglycol mono- and di-esters of 12-hydroxystearic acid and about 30% free polyethylene glycol (available as Solutol HS 15) is used as a solubilizer.

Mixtures of solubilizers may also be used. Examples include, but not limited to, triacetin, triethylcitrate, ethyl oleate, ethyl caprylate, dimethylacetamide, N-methylpyrrolidone, N-hydroxyethylpyrrolidone, polyvinylpyrrolidone, hydroxypropyl methylcellulose, hydroxypropyl cyclodextrins, ethanol, polyethylene glycol 200-100, glycofurol, transcutol, propylene glycol, and dimethyl isosorbide. Particularly preferred solubilizers include sorbitol, glycerol, triacetin, ethyl alcohol, PEG-400, glycofurol and propylene glycol.

The amount of solubilizer that can be included is not particularly limited. The amount of a given solubilizer may be limited to a bioacceptable amount, which may be readily determined by one of skill in the art. In some circumstances, it may be advantageous to include amounts of solubilizers far in excess of bioacceptable amounts, for example to maximize the concentration of the drug, with excess solubilizer removed prior to providing the composition to a subject using conventional techniques, such as distillation or evaporation. Thus, if present, the solubilizer can be in a weight ratio of 10%, 25%, 50%, 100%, or up to about 200% by weight, based on the combined weight of the drug, and other excipients. If desired, very small amounts of solubilizer may also be used, such as 5%, 2%, 1% or even less. Typically, the solubilizer may be present in an amount of about 1% to about 100%, more typically about 5% to about 25% by weight.

The composition can further include one or more pharmaceutically acceptable additives and excipients. Such additives and excipients include, without limitation, detackifiers, anti-foaming agents, buffering agents, polymers, antioxidants, preservatives, chelating agents, viscomodulators, tonicifiers, flavorants, colorants, odorants, opacifiers, suspending agents, binders, fillers, plasticizers, lubricants, and mixtures thereof.

In addition, an acid or a base may be incorporated into the composition to facilitate processing, to enhance stability, or for other reasons. Examples of pharmaceutically acceptable bases include amino acids, amino acid esters, ammonium hydroxide, potassium hydroxide, sodium hydroxide, sodium hydrogen carbonate, aluminum hydroxide, calcium carbonate, magnesium hydroxide, magnesium aluminum silicate, synthetic aluminum silicate, synthetic hydrocalcite, magnesium aluminum hydroxide, diisopropylethylamine, ethanolamine, ethylenediamine, triethanolamine, triethylamine, triisopropanolamine, trimethylamine, tris(hydroxymethyl)aminomethane (TRIS) and the like. Also suitable are bases that are salts of a pharmaceutically acceptable acid, such as acetic acid, acrylic acid, adipic acid, alginic acid, alkanesulfonic acid, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid, and the like. Salts of polyprotic acids, such as sodium phosphate, disodium hydrogen phosphate, and sodium dihydrogen phosphate can also be used. When the base is a salt, the cation can be any convenient and pharmaceutically acceptable cation, such as ammonium, alkali metals, alkaline earth metals, and the like. Example may include, but not limited to, sodium, potassium, lithium, magnesium, calcium and ammonium.

Suitable acids are pharmaceutically acceptable organic or inorganic acids. Examples of suitable inorganic acids include hydrochloric acid, hydrobromic acid, hydriodic acid, sulfuric acid, nitric acid, boric acid, phosphoric acid, and the like. Examples of suitable organic acids include acetic acid, acrylic acid, adipic acid, alginic acid, alkanesulfonic acids, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, methanesulfonic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid and the like.

Pharmaceutical compositions for injection. In some embodiments, the invention provides a pharmaceutical composition for injection containing a compound of the present invention and a pharmaceutical excipient suitable for injection. Components and amounts of agents in the compositions are as described herein.

The forms in which the novel compositions of the present invention may be incorporated for administration by injection include aqueous or oil suspensions, or emulsions, with sesame oil, corn oil, cottonseed oil, or peanut oil, as well as elixirs, mannitol, dextrose, or a sterile aqueous solution, and similar pharmaceutical vehicles.

Aqueous solutions in saline are also conventionally used for injection. Ethanol, glycerol, propylene glycol, liquid polyethylene glycol, and the like (and suitable mixtures thereof), cyclodextrin derivatives, and vegetable oils may also be employed. The proper fluidity can be maintained, for example, by the use of a coating, such as lecithin, for the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like.

Sterile injectable solutions are prepared by incorporating the compound of the present invention in the required amount in the appropriate solvent with various other ingredients as enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, certain desirable methods of preparation are vacuum-drying and freeze-drying techniques which yield a powder of the active ingredient plus any additional desired ingredient from a previously sterile-filtered solution thereof.

Pharmaceutical compositions for topical (e.g., transdermal) delivery. In some embodiments, the invention provides a pharmaceutical composition for transdermal delivery containing a compound of the present invention and at least one pharmaceutical excipient suitable for transdermal delivery.

Compositions of the present invention can be formulated into preparations in solid, semi-solid, or liquid forms suitable for local or topical administration, such as gels, water soluble jellies, creams, lotions, suspensions, foams, powders, slurries, ointments, solutions, oils, pastes, suppositories, sprays, emulsions, saline solutions, dimethylsulfoxide (DMSO)-based solutions. In general, carriers with higher densities are capable of providing an area with a prolonged exposure to the active ingredients. In contrast, a solution formulation may provide more immediate exposure of the active ingredient to the chosen area.

The pharmaceutical compositions also may comprise suitable solid or gel phase carriers or excipients, which are compounds that allow increased penetration of, or assist in the delivery of, therapeutic molecules across the stratum corneum permeability barrier of the skin There are many of these penetration-enhancing molecules known to those trained in the art of topical formulation. Examples of such carriers and excipients include, but are not limited to, humectants (e.g., urea), glycols (e.g., propylene glycol), alcohols (e.g., ethanol), fatty acids (e.g., oleic acid), surfactants (e.g., isopropyl myristate and sodium lauryl sulfate), pyrrolidones, glycerol monolaurate, sulfoxides, terpenes (e.g., menthol), amines, amides, alkanes, alkanols, water, calcium carbonate, calcium phosphate, various sugars, starches, cellulose derivatives, gelatin, and polymers such as polyethylene glycols.

Another exemplary formulation for use in the methods of the present invention employs transdermal delivery devices (“patches”). Such transdermal patches may be used to provide continuous or discontinuous infusion of a compound of the present invention in controlled amounts, either with or without another agent. The construction and use of transdermal patches for the delivery of pharmaceutical agents is well known in the art. See, e.g., U.S. Pat. Nos. 5,023,252, 4,992,445 and 5,001,139. Such patches may be constructed for continuous, pulsatile, or on demand delivery of pharmaceutical agents.

Pharmaceutical compositions for inhalation. Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. Preferably the compositions are administered by the oral or nasal respiratory route for local or systemic effect. Compositions in preferably pharmaceutically acceptable solvents may be nebulized by use of inert gases. Nebulized solutions may be inhaled directly from the nebulizing device or the nebulizing device may be attached to a face mask tent, or intermittent positive pressure breathing machine. Solution, suspension, or powder compositions may be administered, preferably orally or nasally, from devices that deliver the formulation in an appropriate manner.

In some embodiments, the invention provides a pharmaceutical composition for treating ophthalmic disorders. The composition is formulated for ocular administration and it contains an effective amount of one or more polymorphs of the present invention and a pharmaceutical excipient suitable for ocular administration. Pharmaceutical compositions of the invention suitable for ocular administration can be presented as discrete dosage forms, such as drops or sprays each containing a predetermined amount of an active ingredient in a solution, or a suspension in an aqueous or non-aqueous liquid, an oil-in-water emulsion, or a water-in-oil liquid emulsion. Eye drops may be prepared by dissolving the active ingredient in a sterile aqueous solution such as physiological saline, buffering solution, etc., or by combining powder compositions to be dissolved before use. Other vehicles may be chosen, as is known in the art, including but not limited to: balance salt solution, saline solution, water soluble polyethers such as polyethyene glycol, polyvinyls, such as polyvinyl alcohol and povidone, cellulose derivatives such as methylcellulose and hydroxypropyl methylcellulose, petroleum derivatives such as mineral oil and white petrolatum, animal fats such as lanolin, polymers of acrylic acid such as carboxypolymethylene gel, vegetable fats such as peanut oil and polysaccharides such as dextrans, and glycosaminoglycans such as sodium hyaluronate. If desired, additives ordinarily used in the eye drops can be added. Such additives include isotonizing agents (e.g., sodium chloride, etc.), buffer agent (e.g., boric acid, sodium monohydrogen phosphate, sodium dihydrogen phosphate, etc.), preservatives (e.g., benzalkonium chloride, benzethonium chloride, chlorobutanol, etc.), thickeners (e.g., saccharide such as lactose, mannitol, maltose, etc.; e.g., hyaluronic acid or its salt such as sodium hyaluronate, potassium hyaluronate, etc.; e.g., mucopolysaccharide such as chondroitin sulfate, etc.; e.g., sodium polyacrylate, carboxyvinyl polymer, crosslinked polyacrylate, polyvinyl alcohol, polyvinyl pyrrolidone, methyl cellulose, hydroxy propyl methylcellulose, hydroxyethyl cellulose, carboxymethyl cellulose, hydroxy propyl cellulose or other agents known to those skilled in the art).

Other pharmaceutical compositions. Pharmaceutical compositions may also be prepared from compositions described herein and one or more pharmaceutically acceptable excipients suitable for sublingual, buccal, rectal, intraosseous, intraocular, intranasal, epidural, or intraspinal administration. Preparations for such pharmaceutical compositions are well-known in the art. See, e.g., See, e.g., Anderson, Philip O.; Knoben, James E.; Troutman, William G, eds., Handbook of Clinical Drug Data, Tenth Edition, McGraw-Hill, 2002; Pratt and Taylor, eds., Principles of Drug Action, Third Edition, Churchill Livingston, N.Y., 1990; Katzung, ed., Basic and Clinical Pharmacology, Ninth Edition, McGraw Hill, 20037ybg; Goodman and Gilman, eds., The Pharmacological Basis of Therapeutics, Tenth Edition, McGraw Hill, 2001; Remingtons Pharmaceutical Sciences, 20th Ed., Lippincott Williams & Wilkins, 2000; Martindale, The Extra Pharmacopoeia, Thirty-Second Edition (The Pharmaceutical Press, London, 1999); all of which are incorporated by reference herein in their entirety.

Administration of the polymorphs or pharmaceutical composition of the present invention can be effected by any method that enables delivery of the polymorphs to the site of action. These methods include oral routes, intraduodenal routes, parenteral injection (including intravenous, intraarterial, subcutaneous, intramuscular, intravascular, intraperitoneal or infusion), topical (e.g. transdermal application), rectal administration, via local delivery by catheter or stent or through inhalation Polymorphs can also be administered intraadiposally or intrathecally.

The amount of the compound administered will be dependent on the mammal being treated, the severity of the disorder or condition, the rate of administration, the disposition of the compound and the discretion of the prescribing physician. However, an effective dosage is in the range of about 0.001 to about 100 mg per kg body weight per day, preferably about 1 to about 35 mg/kg/day, in single or divided doses. For a 70 kg human, this would amount to about 0.05 to 7 g/day, preferably about 0.05 to about 2.5 g/day. In some instances, dosage levels below the lower limit of the aforesaid range may be more than adequate, while in other cases still larger doses may be employed without causing any harmful side effect, e.g. by dividing such larger doses into several small doses for administration throughout the day.

In some embodiments, a compound of the invention is administered in a single dose. Typically, such administration will be by injection, e.g., intravenous injection, in order to introduce the agent quickly. However, other routes may be used as appropriate. A single dose of a compound of the invention may also be used for treatment of an acute condition.

In some embodiments, a compound of the invention is administered in multiple doses. Dosing may be about once, twice, three times, four times, five times, six times, or more than six times per day. Dosing may be about once a month, once every two weeks, once a week, or once every other day. In another embodiment a compound of the invention and another agent are administered together about once per day to about 6 times per day. In another embodiment the administration of a compound of the invention and an agent continues for less than about 7 days. In yet another embodiment the administration continues for more than about 6, 10, 14, 28 days, two months, six months, or one year. In some cases, continuous dosing is achieved and maintained as long as necessary. In various embodiments, the administration is once weekly.

Administration of the agents of the invention may continue as long as necessary. In some embodiments, an agent of the invention is administered for more than 1, 2, 3, 4, 5, 6, 7, 14, or 28 days. In some embodiments, an agent of the invention is administered for less than 28, 14, 7, 6, 5, 4, 3, 2, or 1 day. In some embodiments, an agent of the invention is administered chronically on an ongoing basis, e.g., for the treatment of chronic effects.

An effective amount of a compound of the invention may be administered in either single or multiple doses by any of the accepted modes of administration of agents having similar utilities, including rectal, buccal, intranasal and transdermal routes, by intra-arterial injection, intravenously, intraperitoneally, parenterally, intramuscularly, subcutaneously, orally, topically, or as an inhalant.

The compositions of the invention may also be delivered via an impregnated or coated device such as a stent, for example, or an artery-inserted cylindrical polymer. Such a method of administration may, for example, aid in the prevention or amelioration of restenosis following procedures such as balloon angioplasty. Without being bound by theory, polymorphs of the invention may slow or inhibit the migration and proliferation of smooth muscle cells in the arterial wall which contribute to restenosis. A compound of the invention may be administered, for example, by local delivery from the struts of a stent, from a stent graft, from grafts, or from the cover or sheath of a stent. In some embodiments, a compound of the invention is admixed with a matrix. Such a matrix may be a polymeric matrix, and may serve to bond the compound to the stent. Polymeric matrices suitable for such use, include, for example, lactone-based polyesters or copolyesters such as polylactide, polycaprolactonglycolide, polyorthoesters, polyanhydrides, polyaminoacids, polysaccharides, polyphosphazenes, poly(ether-ester) copolymers (e.g. PEO-PLLA); polydimethylsiloxane, poly(ethylene-vinylacetate), acrylate-based polymers or copolymers (e.g. polyhydroxyethyl methylmethacrylate, polyvinyl pyrrolidinone), fluorinated polymers such as polytetrafluoroethylene and cellulose esters. Suitable matrices may be nondegrading or may degrade with time, releasing the compound or compounds. Polymorphs of the invention may be applied to the surface of the stent by various methods such as dip/spin coating, spray coating, dip-coating, and/or brush-coating. The polymorphs may be applied in a solvent and the solvent may be allowed to evaporate, thus forming a layer of compound onto the stent. Alternatively, the compound may be located in the body of the stent or graft, for example in microchannels or micropores. When implanted, the compound diffuses out of the body of the stent to contact the arterial wall. Such stents may be prepared by dipping a stent manufactured to contain such micropores or microchannels into a solution of the compound of the invention in a suitable solvent, followed by evaporation of the solvent. Excess drug on the surface of the stent may be removed via an additional brief solvent wash. In yet other embodiments, polymorphs of the invention may be covalently linked to a stent or graft. A covalent linker may be used which degrades in vivo, leading to the release of the compound of the invention. Any bio-labile linkage may be used for such a purpose, such as ester, amide or anhydride linkages Polymorphs of the invention may additionally be administered intravascularly from a balloon used during angioplasty. Extravascular administration of the polymorphs via the pericardia or via advential application of formulations of the invention may also be performed to decrease restenosis.

A variety of stent devices which may be used as described are disclosed, for example, in the following references, all of which are hereby incorporated by reference: U.S. Pat. No. 5,451,233; U.S. Pat. No. 5,040,548; U.S. Pat. No. 5,061,273; U.S. Pat. No. 5,496,346; U.S. Pat. No. 5,292,331; U.S. Pat. No. 5,674,278; U.S. Pat. No. 3,657,744; U.S. Pat. No. 4,739,762; U.S. Pat. No. 5,195,984; U.S. Pat. No. 5,292,331; U.S. Pat. No. 5,674,278; U.S. Pat. No. 5,879,382; U.S. Pat. No. 6,344,053.

The polymorphs of the invention may be administered in dosages. It is known in the art that due to intersubject variability in compound pharmacokinetics, individualization of dosing regimen is necessary for optimal therapy. Dosing for a compound of the invention may be found by routine experimentation in light of the instant disclosure.

The invention also provides kits. The kits include a compound or polymorphs of the present invention as described herein, in suitable packaging, and written material that can include instructions for use, discussion of clinical studies, listing of side effects, and the like. Such kits may also include information, such as scientific literature references, package insert materials, clinical trial results, and/or summaries of these and the like, which indicate or establish the activities and/or advantages of the composition, and/or which describe dosing, administration, side effects, drug interactions, or other information useful to the health care provider. Such information may be based on the results of various studies, for example, studies using experimental animals involving in vivo models and studies based on human clinical trials. The kit may further contain another agent. In some embodiments, the compound of the present invention and the agent are provided as separate compositions in separate containers within the kit. In some embodiments, the compound of the present invention and the agent are provided as a single composition within a container in the kit. Suitable packaging and additional articles for use (e.g., measuring cup for liquid preparations, foil wrapping to minimize exposure to air, and the like) are known in the art and may be included in the kit. Kits described herein can be provided, marketed and/or promoted to health providers, including physicians, nurses, pharmacists, formulary officials, and the like. Kits may also, in some embodiments, be marketed directly to the consumer.

The polymorphs described herein can be used in combination with the agents disclosed herein or other suitable agents, depending on the condition being treated. Hence, in some embodiments the polymorphs of the invention will be co-administered with other agents as described above. When used in combination therapy, the polymorphs described herein may be administered with the second agent simultaneously or separately. This administration in combination can include simultaneous administration of the two agents in the same dosage form, simultaneous administration in separate dosage forms, and separate administration. That is, a compound described herein and any of the agents described above can be formulated together in the same dosage form and administered simultaneously. Alternatively, a compound of the present invention and any of the agents described above can be simultaneously administered, wherein both the agents are present in separate formulations. In another alternative, a compound of the present invention can be administered just followed by and any of the agents described above, or vice versa. In the separate administration protocol, a compound of the present invention and any of the agents described above may be administered a few minutes apart, or a few hours apart, or a few days apart.

### IV. Methods of Treatment

The invention also provides methods of using the compounds or pharmaceutical compositions of the present invention to treat disease conditions, including but not limited to diseases associated with malfunctioning of mTOR or one or more types of PI3 kinase.

The treatment methods provided herein comprise administering to the subject a therapeutically effective amount of a compound of the invention. In one embodiment, the present invention provides a method of treating an inflammation disorder, including autoimmune diseases in a mammal. The method comprises administering to said mammal a therapeutically effective amount of a compound of the present invention, or a pharmaceutically acceptable salt, ester, prodrug, solvate, hydrate or derivative thereof. Examples of autoimmune diseases includes but is not limited to acute disseminated encephalomyelitis (ADEM), Addison's disease, antiphospholipid antibody syndrome (APS), aplastic anemia, autoimmune hepatitis, coeliac disease, Crohn's disease, Diabetes mellitus (type 1), Goodpasture's syndrome, Graves' disease, Guillain-Barré syndrome (GBS), Hashimoto's disease, lupus erythematosus, multiple sclerosis, myasthenia gravis, opsoclonus myoclonus syndrome (OMS), optic neuritis, Ord's thyroiditis, oemphigus, polyarthritis, primary biliary cirrhosis, psoriasis, rheumatoid arthritis, Reiter's syndrome, Takayasu's arteritis, temporal arteritis (also known as “giant cell arteritis”), warm autoimmune hemolytic anemia, Wegener's granulomatosis, alopecia universalis, Chagas' disease, chronic fatigue syndrome, dysautonomia, endometriosis, hidradenitis suppurativa, interstitial cystitis, neuromyotonia, sarcoidosis, scleroderma, ulcerative colitis, vitiligo, and vulvodynia. Other disorders include bone-resorption disorders and thromobsis.

In some embodiments, the method of treating inflammatory or autoimmune diseases comprises administering to a subject (e.g. a mammal) a therapeutically effective amount of one or more compounds of the present invention that selectively inhibit mTOR as compared to all other types of kinases. Such selective inhibition may be advantageous for treating any of the diseases or conditions described herein. For example, selective inhibition may inhibit inflammatory responses associated with inflammatory diseases, autoimmune disease, or diseases related to an undesirable immune response including but not limited to asthma, emphysema, allergy, dermatitis, rhuematoid arthritis, psoriasis, lupus erythematosus, or graft versus host disease. Selective inhibition of mTOR may further provide for a reduction in the inflammatory or undesirable immune response without a concomittant reduction in the ability to reduce a bacterial, viral, and/or fungal infection. Selective inhibition of both mTOR C1/C2 may be advantageous for inhibiting the inflammatory response in the subject to a greater degree than that would be provided for by inhibitors that selectively inhibit mTOR C1 or mTOR C2 alone. In one aspect, one or more of the subject methods are effective in reducing antigen specific antibody production in vivo by about 2-fold, 3-fold, 4-fold, 5-fold, 7.5-fold, 10-fold, 25-fold, 50-fold, 100-fold, 250-fold, 500-fold, 750-fold, or about 1000-fold or more. In another aspect, one or more of the subject methods are effective in reducing antigen specific IgG3 and/or IgGM production in vivo by about 2-fold, 3-fold, 4-fold, 5-fold, 7.5-fold, 10-fold, 25-fold, 50-fold, 100-fold, 250-fold, 500-fold, 750-fold, or about 1000-fold or more.

In one aspect, one of more of the subject methods are effective in ameliorating symptoms associated with rheumatoid arthritis including but not limited to a reduction in the swelling of joints, a reduction in serum anti-collagen levels, and/or a reduction in joint pathology such as bone resorption, cartilage damage, pannus, and/or inflammation. In another aspect, the subject methods are effective in reducing ankle inflammation by at least about 2%, 5%, 10%, 15%, 20%, 25%, 30%, 50%, 60%, or about 75% to 90%. In another aspect, the subject methods are effective in reducing knee inflammation by at least about 2%, 5%, 10%, 15%, 20%, 25%, 30%, 50%, 60%, or about 75% to 90% or more. In still another aspect, the subject methods are effective in reducing serum anti-type II collagen levels by at least about 10%, 12%, 15%, 20%, 24%, 25%, 30%, 35%, 50%, 60%, 75%, 80%, 86%, 87%, or about 90% or more. In another aspect, the subject methods are effective in reducing ankle histopathology scores by about 5%, 10%, 15%, 20%, 25%, 30%, 40%, 50%, 60%, 75%, 80%, 90% or more. In still another aspect, the subject methods are effective in reducing knee histopathology scores by about 5%, 10%, 15%, 20%, 25%, 30%, 40%, 50%, 60%, 75%, 80%, 90% or more.

In other embodiments, the present invention provides methods of using the compounds or pharmaceutical compositions to treat respiratory diseases including but not limited to diseases affecting the lobes of lung, pleural cavity, bronchial tubes, trachea, upper respiratory tract, or the nerves and muscle for breathing. For example, methods are provided to treat obstructive pulmonary disease. Chronic obstructive pulmonary disease (COPD) is an umbrella term for a group of respiratory tract diseases that are characterized by airflow obstruction or limitation. Conditions included in this umbrella term are: chronic bronchitis, emphysema, and bronchiectasis.

In another embodiment, the compounds described herein are used for the treatment of asthma. Also, the compounds or pharmaceutical compositions described herein may be used for the treatment of endotoxemia and sepsis. In one embodiment, the compounds or pharmaceutical compositions described herein are used to for the treatment of rheumatoid arthritis (RA). In yet another embodiment, the compounds or pharmaceutical compositions described herein is used for the treatment of contact or atopic dermatitis. Contact dermatitis includes irritant dermatitis, phototoxic dermatitis, allergic dermatitis, photoallergic dermatitis, contact urticaria, systemic contact-type dermatitis and the like. Irritant dermatitis can occur when too much of a substance is used on the skin of when the skin is sensitive to certain substance. Atopic dermatitis, sometimes called eczema, is a kind of dermatitis, an atopic skin disease.

The invention also relates to a method of treating a hyperproliferative disorder in a mammal that comprises administering to said mammal a therapeutically effective amount of a compound of the present invention, or a pharmaceutically acceptable salt, ester, prodrug, solvate, hydrate or derivative thereof. In some embodiments, said method relates to the treatment of cancer such as acute myeloid leukemia, thymus, brain, lung, squamous cell, skin, eye, retinoblastoma, intraocular melanoma, oral cavity and oropharyngeal, bladder, gastric, stomach, pancreatic, bladder, breast, cervical, head, neck, renal, kidney, liver, ovarian, prostate, colorectal, esophageal, testicular, gynecological, thyroid, CNS, PNS, AIDS-related (e.g. Lymphoma and Kaposi's Sarcoma) or viral-induced cancer. In some embodiments, said method relates to the treatment of a non-cancerous hyperproliferative disorder such as benign hyperplasia of the skin (e.g., psoriasis), restenosis, or prostate (e.g., benign prostatic hypertrophy (BPH)).

The invention also relates to a method of treating diseases related to vasculogenesis or angiogenesis in a mammal that comprises administering to said mammal a therapeutically effective amount of a compound of the present invention, or a pharmaceutically acceptable salt, ester, prodrug, solvate, hydrate or derivative thereof. In some embodiments, said method is for treating a disease selected from the group consisting of tumor angiogenesis, chronic inflammatory disease such as rheumatoid arthritis, atherosclerosis, inflammatory bowel disease, skin diseases such as psoriasis, eczema, and scleroderma, diabetes, diabetic retinopathy, retinopathy of prematurity, age-related macular degeneration, hemangioma, glioma, melanoma, Kaposi's sarcoma and ovarian, breast, lung, pancreatic, prostate, colon and epidermoid cancer.

Patients that can be treated with compounds of the present invention, or pharmaceutically acceptable salt, ester, prodrug, solvate, hydrate or derivative of said compounds, according to the methods of this invention include, for example, patients that have been diagnosed as having psoriasis; restenosis; atherosclerosis; BPH; breast cancer such as a ductal carcinoma in duct tissue in a mammary gland, medullary carcinomas, colloid carcinomas, tubular carcinomas, and inflammatory breast cancer; ovarian cancer, including epithelial ovarian tumors such as adenocarcinoma in the ovary and an adenocarcinoma that has migrated from the ovary into the abdominal cavity; uterine cancer; cervical cancer such as adenocarcinoma in the cervix epithelial including squamous cell carcinoma and adenocarcinomas; prostate cancer, such as a prostate cancer selected from the following: an adenocarcinoma or an adenocarinoma that has migrated to the bone; pancreatic cancer such as epitheliod carcinoma in the pancreatic duct tissue and an adenocarcinoma in a pancreatic duct; bladder cancer such as a transitional cell carcinoma in urinary bladder, urothelial carcinomas (transitional cell carcinomas), tumors in the urothelial cells that line the bladder, squamous cell carcinomas, adenocarcinomas, and small cell cancers; leukemia such as acute myeloid leukemia (AML), acute lymphocytic leukemia, chronic lymphocytic leukemia, chronic myeloid leukemia, hairy cell leukemia, myelodysplasia, myeloproliferative disorders, acute myelogenous leukemia (AML), chronic myelogenous leukemia (CIVIL), mastocytosis, chronic lymphocytic leukemia (CLL), multiple myeloma (MM), and myelodysplastic syndrome (MDS); bone cancer; lung cancer such as non-small cell lung cancer (NSCLC), which is divided into squamous cell carcinomas, adenocarcinomas, and large cell undifferentiated carcinomas, and small cell lung cancer; skin cancer such as basal cell carcinoma, melanoma, squamous cell carcinoma and actinic keratosis, which is a skin condition that sometimes develops into squamous cell carcinoma; eye retinoblastoma; cutaneous or intraocular (eye) melanoma; primary liver cancer (cancer that begins in the liver); kidney cancer; thyroid cancer such as papillary, follicular, medullary and anaplastic; AIDS-related lymphoma such as diffuse large B-cell lymphoma, B-cell immunoblastic lymphoma and small non-cleaved cell lymphoma; Kaposi's Sarcoma; viral-induced cancers including hepatitis B virus (HBV), hepatitis C virus (HCV), and hepatocellular carcinoma; human lymphotropic virus-type 1 (HTLV-1) and adult T-cell leukemia/lymphoma; and human papilloma virus (HPV) and cervical cancer; central nervous system cancers (CNS) such as primary brain tumor, which includes gliomas (astrocytoma, anaplastic astrocytoma, or glioblastoma multiforme), Oligodendroglioma, Ependymoma, Meningioma, Lymphoma, Schwannoma, and Medulloblastoma; peripheral nervous system (PNS) cancers such as acoustic neuromas and malignant peripheral nerve sheath tumor (MPNST) including neurofibromas and schwannomas, malignant fibrous cytoma, malignant fibrous histiocytoma, malignant meningioma, malignant mesothelioma, and malignant mixed Müllerian tumor; oral cavity and oropharyngeal cancer such as, hypopharyngeal cancer, laryngeal cancer, nasopharyngeal cancer, and oropharyngeal cancer; stomach cancer such as lymphomas, gastric stromal tumors, and carcinoid tumors; testicular cancer such as germ cell tumors (GCTs), which include seminomas and nonseminomas, and gonadal stromal tumors, which include Leydig cell tumors and Sertoli cell tumors; thymus cancer such as to thymomas, thymic carcinomas, Hodgkin disease, non-Hodgkin lymphomas carcinoids or carcinoid tumors; rectal cancer; and colon cancer.

Patients that can be treated with compounds of the present invention, or pharmaceutically acceptable salt, ester, prodrug, solvate, hydrate or derivative of said compounds, according to the methods of this invention include, for example, patients that have been diagnosed as having conditions including, but not limited to, acoustic neuroma, adenocarcinoma, adrenal gland cancer, anal cancer, angiosarcoma (e.g., lymphangiosarcoma, lymphangioendotheliosarcoma, hemangiosarcoma), benign monoclonal gammopathy, biliary cancer (e.g., cholangiocarcinoma), bladder cancer, breast cancer (e.g., adenocarcinoma of the breast, papillary carcinoma of the breast, mammary cancer, medullary carcinoma of the breast), brain cancer (e.g., meningioma; glioma, e.g., astrocytoma, oligodendroglioma; medulloblastoma), bronchus cancer, cervical cancer (e.g., cervical adenocarcinoma), choriocarcinoma, chordoma, craniopharyngioma, colorectal cancer (e.g., colon cancer, rectal cancer, colorectal adenocarcinoma), epithelial carcinoma, ependymoma, endotheliosarcoma (e.g., Kaposi's sarcoma, multiple idiopathic hemorrhagic sarcoma), endometrial cancer, esophageal cancer (e.g., adenocarcinoma of the esophagus, Barrett's adenocarinoma), Ewing sarcoma, familiar hypereosinophilia, gastric cancer (e.g., stomach adenocarcinoma), gastrointestinal stromal tumor (GIST), head and neck cancer (e.g., head and neck squamous cell carcinoma, oral cancer (e.g., oral squamous cell carcinoma (OSCC)), heavy chain disease (e.g., alpha chain disease, gamma chain disease, mu chain disease), hemangioblastoma, inflammatory myofibroblastic tumors, immunocytic amyloidosis, kidney cancer (e.g., nephroblastoma a.k.a. Wilms' tumor, renal cell carcinoma), liver cancer (e.g., hepatocellular cancer (HCC), malignant hepatoma), lung cancer (e.g., bronchogenic carcinoma, small cell lung cancer (SCLC), non-small cell lung cancer (NSCLC), adenocarcinoma of the lung), leukemia (e.g., acute lymphocytic leukemia (ALL), which includes B-lineage ALL and T-lineage ALL, chronic lymphocytic leukemia (CLL), prolymphocytic leukemia (PLL), hairy cell leukemia (FILL) and Waldenstrom's macroglobulinemia (WM); peripheral T cell lymphomas (PTCL), adult T cell leukemia/lymphoma (ATL), cutaneous T-cell lymphoma (CTCL), large granular lymphocytic leukemia (LGF), Hodgkin's disease and Reed-Stemberg disease; acute myelocytic leukemia (AML), chronic myelocytic leukemia (CML), chronic lymphocytic leukemia (CLL)), lymphoma (e.g., Hodgkin lymphoma (HL), non-Hodgkin lymphoma (NHL), follicular lymphoma, diffuse large B-cell lymphoma (DLBCL), mantle cell lymphoma (MCL)), leiomyosarcoma (LMS), mastocytosis (e.g., systemic mastocytosis), multiple myeloma (MM), myelodysplastic syndrome (MDS), mesothelioma, myeloproliferative disorder (MPD) (e.g., polycythemia Vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)), neuroblastoma, neurofibroma (e.g., neurofibromatosis (NF) type 1 or type 2, schwannomatosis), neuroendocrine cancer (e.g., gastroenteropancreatic neuroendoctrine tumor (GEP-NET), carcinoid tumor), osteosarcoma, ovarian cancer (e.g., cystadenocarcinoma, ovarian embryonal carcinoma, ovarian adenocarcinoma), Paget's disease of the vulva, Paget's disease of the penis, papillary adenocarcinoma, pancreatic cancer (e.g., pancreatic andenocarcinoma, intraductal papillary mucinous neoplasm (IPMN)), pinealoma, primitive neuroectodermal tumor (PNT), prostate cancer (e.g., prostate adenocarcinoma), rhabdomyosarcoma, retinoblastoma, salivary gland cancer, skin cancer (e.g., squamous cell carcinoma (SCC), keratoacanthoma (KA), melanoma, basal cell carcinoma (BCC)), small bowel cancer (e.g., appendix cancer), soft tissue sarcoma (e.g., malignant fibrous histiocytoma (MFH), liposarcoma, malignant peripheral nerve sheath tumor (MPNST), chondrosarcoma, fibrosarcoma, myxosarcoma), sebaceous gland carcinoma, sweat gland carcinoma, synovioma, testicular cancer (e.g., seminoma, testicular embryonal carcinoma), thyroid cancer (e.g., papillary carcinoma of the thyroid, papillary thyroid carcinoma (PTC), medullary thyroid cancer), and Waldenström's macroglobulinemia.

The invention also relates to a method of treating diabetes in a mammal that comprises administering to said mammal a therapeutically effective amount of a compound of the present invention, or a pharmaceutically acceptable salt, ester, prodrug, solvate, hydrate or derivative thereof.

In addition, the compounds described herein may be used to treat acne.

In addition, the compounds described herein may be used for the treatment of arteriosclerosis, including atherosclerosis. Arteriosclerosis is a general term describing any hardening of medium or large arteries. Atherosclerosis is a hardening of an artery specifically due to an atheromatous plaque.

Further the compounds described herein may be used for the treatment of glomerulonephritis. Glomerulonephritis is a primary or secondary autoimmune renal disease characterized by inflammation of the glomeruli. It may be asymptomatic, or present with hematuria and/or proteinuria. There are many recognized types, divided in acute, subacute or chronic glomerulonephritis. Causes are infectious (bacterial, viral or parasitic pathogens), autoimmune or paraneoplastic.

Additionally, the compounds described herein may be used for the treatment of bursitis, lupus, acute disseminated encephalomyelitis (ADEM), Addison's disease, antiphospholipid antibody syndrome (APS), aplastic anemia, autoimmune hepatitis, coeliac disease, Crohn's disease, diabetes mellitus (type 1), goodpasture's syndrome, graves' disease, guillain-barré syndrome (GBS), hashimoto's disease, inflammatory bowel disease, lupus erythematosus, myasthenia gravis, opsoclonus myoclonus syndrome (OMS), optic neuritis, ord's thyroiditis, ostheoarthritis, uveoretinitis, pemphigus, polyarthritis, primary biliary cirrhosis, reiter's syndrome, takayasu's arteritis, temporal arteritis, warm autoimmune hemolytic anemia, wegener's granulomatosis, alopecia universalis, chagas' disease, chronic fatigue syndrome, dysautonomia, endometriosis, hidradenitis suppurativa, interstitial cystitis, neuromyotonia, sarcoidosis, scleroderma, ulcerative colitis, vitiligo, vulvodynia, appendicitis, arteritis, arthritis, blepharitis, bronchiolitis, bronchitis, cervicitis, cholangitis, cholecystitis, chorioamnionitis, colitis, conjunctivitis, cystitis, dacryoadenitis, dermatomyositis, endocarditis, endometritis, enteritis, enterocolitis, epicondylitis, epididymitis, fasciitis, fibrositis, gastritis, gastroenteritis, gingivitis, hepatitis, hidradenitis, ileitis, iritis, laryngitis, mastitis, meningitis, myelitis, myocarditis, myositis, nephritis, omphalitis, oophoritis, orchitis, osteitis, otitis, pancreatitis, parotitis, pericarditis, peritonitis, pharyngitis, pleuritis, phlebitis, pneumonitis, proctitis, prostatitis, pyelonephritis, rhinitis, salpingitis, sinusitis, stomatitis, synovitis, tendonitis, tonsillitis, uveitis, vaginitis, vasculitis, or vulvitis.

Further, the compounds of the invention may be used for the treatment of perennial allergic rhinitis, Mesenteritis, Peritonitis, Acrodermatitis, Angiodermatitis, Atopic dermatitis, Contact dermatitis, Eczema, Erythema multiforme, Intertrigo, Stevens Johnson syndrome, Toxic epidermal necrolysis, Skin allergy, Severe allergic reaction/anaphylaxis, Allergic granulomatosis, Wegener granulomatosis, Allergic conjunctivitis, Chorioretinitis, Conjunctivitis, Infectious keratoconjunctivitis, Keratoconjunctivitis, Ophthalmia neonatorum, Trachoma, Uveitis, Ocular inflammation, Blepharoconjunctivitis, Mastitis, Gingivitis, Pericoronitis, Pharyngitis, Rhinopharyngitis, Sialadenitis, Musculoskeletal system inflammation, Adult onset Stills disease, Behcets disease, Bursitis, Chondrocalcinosis, Dactylitis, Felty syndrome, Gout, Infectious arthritis, Lyme disease, Inflammatory osteoarthritis, Periarthritis, Reiter syndrome, Ross River virus infection, Acute Respiratory, Distress Syndrome, Acute bronchitis, Acute sinusitis, Allergic rhinitis, Asthma, Severe refractory asthma, Pharyngitis, Pleurisy, Rhinopharyngitis, Seasonal allergic rhinitis, Sinusitis, Status asthmaticus, Tracheobronchitis, Rhinitis, Serositis, Meningitis, Neuromyelitis optica, Poliovirus infection, Alport syndrome, Balanitis, Epididymitis, Epididymo orchitis, Focal segmental, Glomerulosclerosis, Glomerulonephritis, IgA Nephropathy (Berger's Disease), Orchitis, Parametritis, Pelvic inflammatory disease, Prostatitis, Pyelitis, Pyelocystitis, Pyelonephritis, Wegener granulomatosis, Hyperuricemia, Aortitis, Arteritis, Chylopericarditis, Dressler syndrome, Endarteritis, Endocarditis, Extracranial temporal arteritis, HIV associated arteritis, Intracranial temporal arteritis, Kawasaki disease, Lymphangiophlebitis, Mondor disease, Periarteritis, or Pericarditis.

In other aspects, the compounds of the invention are used for the treatment of Autoimmune hepatitis, Jejunitis, Mesenteritis, Mucositis, Non alcoholic steatohepatitis, Non viral hepatitis, Autoimmune pancreatitis, Perihepatitis, Peritonitis, Pouchitis, Proctitis, Pseudomembranous colitis, Rectosigmoiditis, Salpingoperitonitis, Sigmoiditis, Steatohepatitis, Ulcerative colitis, Churg Strauss syndrome, Ulcerative proctitis, Irritable bowel syndrome, Gastrointestinal inflammation, Acute enterocolitis, Anusitis, Balser necrosis, Cholecystitis, Colitis, Crohns disease, Diverticulitis, Enteritis, Enterocolitis, Enterohepatitis, Eosinophilic esophagitis, Esophagitis, Gastritis, Hemorrhagic enteritis, Hepatitis, Hepatitis virus infection, Hepatocholangitis, Hypertrophic gastritis, Ileitis, Ileocecitis, Sarcoidosis, Inflammatory bowel disease, Ankylosing spondylitis, Rheumatoid arthritis, Juvenile rheumatoid arthritis, Psoriasis, Psoriatic arthritis, Lupus (cutaneous/systemic/nephritis), AIDS, Agammaglobulinemia, AIDS related complex, Brutons disease, Chediak Higashi syndrome, Common variable immunodeficiency, DiGeorge syndrome, Dysgammaglobulinemia, Immunoglobulindeficiency, Job syndrome, Nezelof syndrome, Phagocyte bactericidal disorder, Wiskott Aldrich syndrome, Asplenia, Elephantiasis, Hypersplenism, Kawasaki disease, Lymphadenopathy, Lymphedema, Lymphocele, Nonne Milroy Meige syndrome, Spleen disease, Splenomegaly, Thymoma, Thymus disease, Perivasculitis, Phlebitis, Pleuropericarditis, Polyarteritis nodosa, Vasculitis, Takayasus arteritis, Temporal arteritis, Thromboangiitis, Thromboangiitis obliterans, Thromboendocarditis, Thrombophlebitis, or COPD.

The invention also relates to a method of treating a cardiovascular disease in a mammal that comprises administering to said mammal a therapeutically effective amount of a compound of the present invention, or a pharmaceutically acceptable salt, ester, prodrug, solvate, hydrate or derivative thereof. Examples of cardiovascular conditions include, but are not limited to, atherosclerosis, restenosis, vascular occlusion and carotid obstructive disease.

In another aspect, the present invention provides methods of disrupting the function of a leukocyte or disrupting a function of an osteoclast. The method includes contacting the leukocyte or the osteoclast with a function disrupting amount of a compound of the invention.

In another aspect of the present invention, methods are provided for treating ophthalmic disease by administering one or more of the subject compounds or pharmaceutical compositions to the eye of a subject.

Methods are further provided for administering the compounds of the present invention via eye drop, intraocular injection, intravitreal injection, topically, or through the use of a drug eluting device, microcapsule, implant, or microfluidic device. In some cases, the compounds of the present invention are administered with a carrier or excipient that increases the intraocular penetrance of the compound such as an oil and water emulsion with colloid particles having an oily core surrounded by an interfacial film.

The invention further provides methods of modulating kinase activity by contacting a kinase with an amount of a compound of the invention sufficient to modulate the activity of the kinase. Modulate can be inhibiting or activating kinase activity. In some embodiments, the invention provides methods of inhibiting kinase activity by contacting a kinase with an amount of a compound of the invention sufficient to inhibit the activity of the kinase. In some embodiments, the invention provides methods of inhibiting kinase activity in a solution by contacting said solution with an amount of a compound of the invention sufficient to inhibit the activity of the kinase in said solution. In some embodiments, the invention provides methods of inhibiting kinase activity in a cell by contacting said cell with an amount of a compound of the invention sufficient to inhibit the activity of the kinase in said cell. In some embodiments, the invention provides methods of inhibiting kinase activity in a tissue by contacting said tissue with an amount of a compound of the invention sufficient to inhibit the activity of the kinase in said tissue. In some embodiments, the invention provides methods of inhibiting kinase activity in an organism by contacting said organism with an amount of a compound of the invention sufficient to inhibit the activity of the kinase in said organism. In some embodiments, the invention provides methods of inhibiting kinase activity in an animal by contacting said animal with an amount of a compound of the invention sufficient to inhibit the activity of the kinase in said animal. In some embodiments, the invention provides methods of inhibiting kinase activity in a mammal by contacting said mammal with an amount of a compound of the invention sufficient to inhibit the activity of the kinase in said mammal. In some embodiments, the invention provides methods of inhibiting kinase activity in a human by contacting said human with an amount of a compound of the invention sufficient to inhibit the activity of the kinase in said human. In some embodiments, the % of kinase activity after contacting a kinase with a compound of the invention is less than 1, 5, 10, 20, 30, 40, 50, 60, 70, 80, 90, 95, or 99% of the kinase activity in the absence of said contacting step.

In some embodiments, one or more compounds of the invention selectively inhibits both mTor activity with an IC₅₀ value of about 100 nM, 50 nM, 10 nM, 5 nM, 100 pM, 10 pM or even 1 pM, or less as ascertained in an in vitro kinase assay.

In some embodiments, one or more compounds or polymorphs of the invention inhibit phosphorylation of Akt (S473) and Akt (T308) more effectively than rapamycin when tested at a comparable molar concentration in an in vitro kinase assay.

In some embodiments, one or more polymorphs or compounds of the invention compete with ATP for binding to ATP-binding site on mTorC1 and/or mTorC2.

### V. Combination Treatment

The present invention also provides methods for combination therapies in which an agent known to modulate other pathways, or other components of the same pathway, or even overlapping sets of target enzymes are used in combination with a compound of the present invention, or a pharmaceutically acceptable salt, ester, prodrug, solvate, hydrate or derivative thereof. In one aspect, such therapy includes but is not limited to the combination of the subject compound with chemotherapeutic agents, therapeutic antibodies, and radiation treatment, to provide a synergistic or additive therapeutic effect.

In one aspect, the compounds or pharmaceutical compositions of the present invention may present synergistic or additive efficacy when administered in combination with agents that inhibit IgE production or activity. Such combination can reduce the undesired effect of high level of IgE associated with the use of one or more inhibitors, if such effect occurs. This may be particularly useful in treatment of autoimmune and inflammatory disorders (AIID) such as rheumatoid arthritis. Additionally, the administration of inhibitors of the present invention in combination with inhibitors of PI3Kα, PI3Kδ, or PI3Kδ/γ may also exhibit synergy through enhanced inhibition of the PI3K pathway.

Agents that inhibit IgE production are known in the art and they include but are not limited to one or more of TEI-9874, 2-(4-(6-cyclohexyloxy-2-naphtyloxy)phenylacetamide)benzoic acid, rapamycin, rapamycin analogs (i.e. rapalogs), TORC1 inhibitors, TORC2 inhibitors, and any other compounds that inhibit mTORC1 and mTORC2. Agents that inhibit IgE activity include, for example, anti-IgE antibodies such as for example Omalizumab and TNX-901.

For treatment of autoimmune diseases, the subject compounds or pharmaceutical compositions can be used in combination with commonly prescribed drugs including but not limited to Enbrel®, Remicade®, Humira®, Avonex®, and Rebif®. For treatment of respiratory diseaseses, the subject compounds or pharmaceutical compositions can be administered in combination with commonly prescribed drugs including but not limited to Xolair®, Advair®, Singulair®, and Spiriva®.

The compounds of the invention may be formulated or administered in conjunction with other agents that act to relieve the symptoms of inflammatory conditions such as encephalomyelitis, asthma, and the other diseases described herein. These agents include non-steroidal anti-inflammatory drugs (NSAIDs), e.g. acetylsalicylic acid; ibuprofen; naproxen; indomethacin; nabumetone; tolmetin; etc. Corticosteroids are used to reduce inflammation and suppress activity of the immune system. The most commonly prescribed drug of this type is Prednisone. Chloroquine (Aralen) or hydroxychloroquine (Plaquenil) may also be very useful in some individuals with lupus. They are most often prescribed for skin and joint symptoms of lupus. Azathioprine (Imuran) and cyclophosphamide (Cytoxan) suppress inflammation and tend to suppress the immune system. Other agents, e.g. methotrexate and cyclosporin are used to control the symptoms of lupus. Anticoagulants are employed to prevent blood from clotting rapidly. They range from aspirin at very low dose which prevents platelets from sticking, to heparin/coumadin. Other compounds used in the treatment of lupus include belimumab (Benlysta®).

In another one aspect, this invention also relates to a pharmaceutical composition for inhibiting abnormal cell growth in a mammal which comprises an amount of a compound of the present invention, or a pharmaceutically acceptable salt, ester, prodrug, solvate, hydrate or derivative thereof, in combination with an amount of an anti-cancer agent (e.g. a biotherapeutic chemotherapeutic agent). Many chemotherapeutics are presently known in the art and can be used in combination with the compounds of the invention. Other cancer therapies can also be used in combination with the compounds of the invention and include, but are not limited to, surgery and surgical treatments, and radiation therapy.

In some embodiments, the chemotherapeutic is selected from the group consisting of mitotic inhibitors, alkylating agents, anti-metabolites, intercalating antibiotics, growth factor inhibitors, cell cycle inhibitors, enzymes, topoisomerase inhibitors, biological response modifiers, anti-hormones, angiogenesis inhibitors, and anti-androgens. Non-limiting examples are chemotherapeutic agents, cytotoxic agents, and non-peptide small molecules such as Gleevec (Imatinib Mesylate), Velcade (bortezomib), Casodex (bicalutamide), Iressa (gefitinib), and Adriamycin as well as a host of chemotherapeutic agents. Non-limiting examples of chemotherapeutic agents include alkylating agents such as thiotepa and cyclosphosphamide (CYTOXAN™); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carminomycin, carzinophilin, Casodex™, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elfomithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK®; razoxane; sizofiran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; taxanes, e.g. paclitaxel (TAXOL™, Bristol-Myers Squibb Oncology, Princeton, N.J.) and docetaxel (TAXOTERE™, Rhone-Poulenc Rorer, Antony, France); retinoic acid; esperamicins; capecitabine; and pharmaceutically acceptable salts, acids or derivatives of any of the above. Also included as suitable chemotherapeutic cell conditioners are anti-hormonal agents that act to regulate or inhibit hormone action on tumors such as anti-estrogens including for example tamoxifen (Nolvadex™), raloxifene, aromatase inhibiting 4(5)-imidazoles, 4-hydroxytamoxifen, trioxifene, keoxifene, LY 117018, onapristone, and toremifene (Fareston); and anti-androgens such as flutamide, nilutamide, bicalutamide, leuprolide, and goserelin; chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; camptothecin-11 (CPT-11); topoisomerase inhibitor RFS 2000; difluoromethylornithine (DMFO). Where desired, the compounds or pharmaceutical composition of the present invention can be used in combination with commonly prescribed anti-cancer drugs such as Herceptin®, Avastin®, Erbitux®, Rituxan®, Taxol®, Arimidex®, Taxotere®, and Velcade®.

Other chemotherapeutic agents include, but are not limited to, anti-estrogens (e.g. tamoxifen, raloxifene, and megestrol), LHRH agonists (e.g. goscrclin and leuprolide), anti-androgens (e.g. flutamide and bicalutamide), photodynamic therapies (e.g. vertoporfin (BPD-MA), phthalocyanine, photosensitizer Pc4, and demethoxy-hypocrellin A (2BA-2-DMHA)), nitrogen mustards (e.g. cyclophosphamide, ifosfamide, trofosfamide, chlorambucil, estramustine, and melphalan), nitrosoureas (e.g. carmustine (BCNU) and lomustine (CCNU)), alkylsulphonates (e.g. busulfan and treosulfan), triazenes (e.g. dacarbazine, temozolomide), platinum containing compounds (e.g. cisplatin, carboplatin, oxaliplatin), vinca alkaloids (e.g. vincristine, vinblastine, vindesine, and vinorelbine), taxoids (e.g. paclitaxel or a paclitaxel equivalent such as nanoparticle albumin-bound paclitaxel (Abraxane), docosahexaenoic acid bound-paclitaxel (DHA-paclitaxel, Taxoprexin), polyglutamate bound-paclitaxel (PG-paclitaxel, paclitaxel poliglumex, CT-2103, XYOTAX), the tumor-activated prodrug (TAP) ANG1005 (Angiopep-2 bound to three molecules of paclitaxel), paclitaxel-EC-1 (paclitaxel bound to the erbB2-recognizing peptide EC-1), and glucose-conjugated paclitaxel, e.g., 2′-paclitaxel methyl 2-glucopyranosyl succinate; docetaxel, taxol), epipodophyllins (e.g. etoposide, etoposide phosphate, teniposide, topotecan, 9-aminocamptothecin, camptoirinotecan, irinotecan, crisnatol, mytomycin C), anti-metabolites, DHFR inhibitors (e.g. methotrexate, dichloromethotrexate, trimetrexate, edatrexate), IMP dehydrogenase inhibitors (e.g. mycophenolic acid, tiazofurin, ribavirin, and EICAR), ribonuclotide reductase inhibitors (e.g. hydroxyurea and deferoxamine), uracil analogs (e.g. 5-fluorouracil (5-FU), floxuridine, doxifluridine, ratitrexed, tegafur-uracil, capecitabine), cytosine analogs (e.g. cytarabine (ara C), cytosine arabinoside, and fludarabine), purine analogs (e.g. mercaptopurine and Thioguanine), Vitamin D3 analogs (e.g. EB 1089, CB 1093, and KH 1060), isoprenylation inhibitors (e.g. lovastatin), dopaminergic neurotoxins (e.g. 1-methyl-4-phenylpyridinium ion), cell cycle inhibitors (e.g. staurosporine), actinomycin (e.g. actinomycin D, dactinomycin), bleomycin (e.g. bleomycin A2, bleomycin B2, peplomycin), anthracycline (e.g. daunorubicin, doxorubicin, pegylated liposomal doxorubicin, idarubicin, epirubicin, pirarubicin, zorubicin, mitoxantrone), MDR inhibitors (e.g. verapamil), Ca2+ ATPase inhibitors (e.g. thapsigargin), imatinib, thalidomide, lenalidomide, tyrosine kinase inhibitors (e.g., axitinib (AG013736), bosutinib (SKI-606), cediranib (RECENTIN™, AZD2171), dasatinib (SPRYCEL®, BMS-354825), erlotinib (TARCEVA®), gefitinib (IRESSA®), imatinib (Gleevec®, CGP57148B, STI-571), lapatinib (TYKERB®, TYVERB®), lestaurtinib (CEP-701), neratinib (HKI-272), nilotinib (TASIGNA®), semaxanib (semaxinib, SU5416), sunitinib (SUTENT®, SU11248), toceranib (PALLADIA®), vandetanib (ZACTIMA®, ZD6474), vatalanib (PTK787, PTK/ZK), trastuzumab (HERCEPTIN®), bevacizumab (AVASTIN®), rituximab (RITUXAN®), cetuximab (ERBITUX®), panitumumab (VECTIBIX®), ranibizumab (Lucentis®), nilotinib (TASIGNA®), sorafenib (NEXAVAR®), everolimus (AFINITOR®), alemtuzumab (CAMPATH®), gemtuzumab ozogamicin (MYLOTARG®), temsirolimus (TORISEL®), ENMD-2076, PCI-32765, AC220, dovitinib lactate (TKI258, CHIR-258), BIBW 2992 (TOVOK™), SGX523, PF-04217903, PF-02341066, PF-299804, BMS-777607, ABT-869, MP470, BIBF 1120 (VARGATEF®), AP24534, JNJ-26483327, MGCD265, DCC-2036, BMS-690154, CEP-11981, tivozanib (AV-951), OSI-930, MM-121, XL-184, XL-647, and/or XL228), proteasome inhibitors (e.g., bortezomib (Velcade)), mTOR inhibitors (e.g., rapamycin, temsirolimus (CCI-779), everolimus (RAD-001), ridaforolimus, AP23573 (Ariad), AZD8055 (AstraZeneca), BEZ235 (Novartis), BGT226 (Norvartis), XL765 (Sanofi Aventis), PF-4691502 (Pfizer), GDC0980 (Genetech), SF1126 (Semafoe) and OSI-027 (OSI)), oblimersen, gemcitabine, carminomycin, leucovorin, pemetrexed, cyclophosphamide, dacarbazine, procarbizine, prednisolone, dexamethasone, campathecin, plicamycin, asparaginase, aminopterin, methopterin, porfiromycin, melphalan, leurosidine, leurosine, chlorambucil, trabectedin, procarbazine, discodermolide, carminomycin, aminopterin, and hexamethyl melamine.

Exemplary biotherapeutic agents include, but are not limited to, interferons, cytokines (e.g., tumor necrosis factor, interferon α, interferon γ), vaccines, hematopoietic growth factors, monoclonal serotherapy, immunostimulants and/or immunodulatory agents (e.g., IL-1, 2, 4, 6, or 12), immune cell growth factors (e.g., GM-CSF) and antibodies (e.g. Herceptin (trastuzumab), T-DM1, AVASTIN (bevacizumab), ERBITUX (cetuximab), Vectibix (panitumumab), Rituxan (rituximab), Bexxar (tositumomab)).

This invention further relates to a method for using the compounds or pharmaceutical composition in combination with radiation therapy in inhibiting abnormal cell growth or treating the hyperproliferative disorder in the mammal. Techniques for administering radiation therapy are known in the art, and these techniques can be used in the combination therapy described herein. The administration of the compound of the invention in this combination therapy can be determined as described herein.

Radiation therapy can be administered through one of several methods, or a combination of methods, including without limitation external-beam therapy, internal radiation therapy, implant radiation, stereotactic radiosurgery, systemic radiation therapy, radiotherapy and permanent or temporary interstitial brachytherapy. The term “brachytherapy,” as used herein, refers to radiation therapy delivered by a spatially confined radioactive material inserted into the body at or near a tumor or other proliferative tissue disease site. The term is intended without limitation to include exposure to radioactive isotopes (e.g. At-211, I-131, I-125, Y-90, Re-186, Re-188, Sm-153, Bi-212, P-32, and radioactive isotopes of Lu). Suitable radiation sources for use as a cell conditioner of the present invention include both solids and liquids. By way of non-limiting example, the radiation source can be a radionuclide, such as I-125, I-131, Yb-169, Ir-192 as a solid source, I-125 as a solid source, or other radionuclides that emit photons, beta particles, gamma radiation, or other therapeutic rays. The radioactive material can also be a fluid made from any solution of radionuclide(s), e.g., a solution of I-125 or I-131, or a radioactive fluid can be produced using a slurry of a suitable fluid containing small particles of solid radionuclides, such as Au-198, Y-90. Moreover, the radionuclide(s) can be embodied in a gel or radioactive micro spheres.

Without being limited by any theory, the compounds of the present invention can render abnormal cells more sensitive to treatment with radiation for purposes of killing and/or inhibiting the growth of such cells. Accordingly, this invention further relates to a method for sensitizing abnormal cells in a mammal to treatment with radiation which comprises administering to the mammal an amount of a compound of the present invention or pharmaceutically acceptable salt, ester, prodrug, solvate, hydrate or derivative thereof, which amount is effective is sensitizing abnormal cells to treatment with radiation. The amount of the compound, salt, or solvate in this method can be determined according to the means for ascertaining effective amounts of such compounds described herein.

The compounds or pharmaceutical compositions of the present invention can be used in combination with an amount of one or more substances selected from anti-angiogenesis agents, signal transduction inhibitors, and antiproliferative agents.

Anti-angiogenesis agents, such as MMP-2 (matrix-metalloprotienase 2) inhibitors, MMP-9 (matrix-metalloprotienase 9) inhibitors, and COX-11 (cyclooxygenase 11) inhibitors, can be used in conjunction with a compound of the present invention and pharmaceutical compositions described herein. Examples of useful COX-II inhibitors include CELEBREX™ (alecoxib), valdecoxib, and rofecoxib. Examples of useful matrix metalloproteinase inhibitors are described in WO 96/33172 (published Oct. 24, 1996), WO 96/27583 (published Mar. 7, 1996), European Patent Application No. 97304971.1 (filed Jul. 8, 1997), European Patent Application No. 99308617.2 (filed Oct. 29, 1999), WO 98/07697 (published Feb. 26, 1998), WO 98/03516 (published Jan. 29, 1998), WO 98/34918 (published Aug. 13, 1998), WO 98/34915 (published Aug. 13, 1998), WO 98/33768 (published Aug. 6, 1998), WO 98/30566 (published Jul. 16, 1998), European Patent Publication 606,046 (published Jul. 13, 1994), European Patent Publication 931, 788 (published Jul. 28, 1999), WO 90/05719 (published May 31, 1990), WO 99/52910 (published Oct. 21, 1999), WO 99/52889 (published Oct. 21, 1999), WO 99/29667 (published Jun. 17, 1999), PCT International Application No. PCT/IB98/01113 (filed Jul. 21, 1998), European Patent Application No. 99302232.1 (filed Mar. 25, 1999), Great Britain Patent Application No. 9912961.1 (filed Jun. 3, 1999), U.S. Provisional Application No. 60/148,464 (filed Aug. 12, 1999), U.S. Pat. No. 5,863,949 (issued Jan. 26, 1999), U.S. Pat. No. 5,861,510 (issued Jan. 19, 1999), and European Patent Publication 780,386 (published Jun. 25, 1997), all of which are incorporated herein in their entireties by reference. Preferred MMP-2 and MMP-9 inhibitors are those that have little or no activity inhibiting MMP-1. More preferred, are those that selectively inhibit MMP-2 and/or AMP-9 relative to the other matrix-metalloproteinases (i.e., MAP-1, MMP-3, MMP-4, MMP-5, MMP-6, MMP-7, MMP-8, MMP-10, MMP-11, MMP-12, and MMP-13). Some specific examples of MMP inhibitors useful in the present invention are AG-3340, RO 32-3555, and RS 13-0830.

The invention also relates to a method of and to a pharmaceutical composition of treating a cardiovascular disease in a mammal which comprises an amount of a compound of the present invention, or a pharmaceutically acceptable salt, ester, prodrug, solvate, hydrate or derivative thereof, or an isotopically-labeled derivative thereof, and an amount of one or more therapeutic agents use for the treatment of cardiovascular diseases.

Examples for use in cardiovascular disease applications are anti-thrombotic agents, e.g., prostacyclin and salicylates, thrombolytic agents, e.g., streptokinase, urokinase, tissue plasminogen activator (TPA) and anisoylated plasminogen-streptokinase activator complex (APSAC), anti-platelets agents, e.g., acetyl-salicylic acid (ASA) and clopidrogel, vasodilating agents, e.g., nitrates, calcium channel blocking drugs, anti-proliferative agents, e.g., colchicine and alkylating agents, intercalating agents, growth modulating factors such as interleukins, transformation growth factor-beta and congeners of platelet derived growth factor, monoclonal antibodies directed against growth factors, anti-inflammatory agents, both steroidal and non-steroidal, and other agents that can modulate vessel tone, function, arteriosclerosis, and the healing response to vessel or organ injury post intervention. Antibiotics can also be included in combinations or coatings comprised by the invention. Moreover, a coating can be used to effect therapeutic delivery focally within the vessel wall. By incorporation of the active agent in a swellable polymer, the active agent will be released upon swelling of the polymer.

The compounds describe herein may be formulated or administered in conjunction with liquid or solid tissue barriers also known as lubricants. Examples of tissue barriers include, but are not limited to, polysaccharides, polyglycans, seprafilm, interceed and hyaluronic acid.

Medicaments which may be administered in conjunction with the compounds described herein include any suitable drugs usefully delivered by inhalation for example, analgesics, e.g. codeine, dihydromorphine, ergotamine, fentanyl or morphine; anginal preparations, e.g. diltiazem; antiallergics, e.g. cromoglycate, ketotifen or nedocromil; anti-infectives, e.g. cephalosporins, penicillins, streptomycin, sulphonamides, tetracyclines or pentamidine; antihistamines, e.g. methapyrilene; anti-inflammatories, e.g. beclomethasone, flunisolide, budesonide, tipredane, triamcinolone acetonide or fluticasone; antitussives, e.g. noscapine; bronchodilators, e.g. ephedrine, adrenaline, fenoterol, formoterol, isoprenaline, metaproterenol, phenylephrine, phenylpropanolamine, pirbuterol, reproterol, rimiterol, salbutamol, salmeterol, terbutalin, isoetharine, tulobuterol, orciprenaline or (−)-4-amino-3,5-dichloro-α-[[[6-[2-(2-pyridinyl)ethoxy]hexyl]-amino]methyl]benzenemethanol; diuretics, e.g. amiloride; anticholinergics e.g. ipratropium, atropine or oxitropium; hormones, e.g. cortisone, hydrocortisone or prednisolone; xanthines e.g. aminophylline, choline theophyllinate, lysine theophyllinate or theophylline; and therapeutic proteins and peptides, e.g. insulin or glucagon. It will be clear to a person skilled in the art that, where appropriate, the medicaments may be used in the form of salts (e.g. as alkali metal or amine salts or as acid addition salts) or as esters (e.g. lower alkyl esters) or as solvates (e.g. hydrates) to optimize the activity and/or stability of the medicament.

Other exemplary therapeutic agents useful for a combination therapy include but are not limited to agents as described above, radiation therapy, hormone antagonists, hormones and their releasing factors, thyroid and antithyroid drugs, estrogens and progestins, androgens, adrenocorticotropic hormone; adrenocortical steroids and their synthetic analogs; inhibitors of the synthesis and actions of adrenocortical hormones, insulin, oral hypoglycemic agents, and the pharmacology of the endocrine pancreas, agents affecting calcification and bone turnover: calcium, phosphate, parathyroid hormone, vitamin D, calcitonin, vitamins such as water-soluble vitamins, vitamin B complex, ascorbic acid, fat-soluble vitamins, vitamins A, K, and E, growth factors, cytokines, chemokines, muscarinic receptor agonists and antagonists; anticholinesterase agents; agents acting at the neuromuscular junction and/or autonomic ganglia; catecholamines, sympathomimetic drugs, and adrenergic receptor agonists or antagonists; and 5-hydroxytryptamine (5-HT, serotonin) receptor agonists and antagonists.

Therapeutic agents can also include agents for pain and inflammation such as histamine and histamine antagonists, bradykinin and bradykinin antagonists, 5-hydroxytryptamine (serotonin), lipid substances that are generated by biotransformation of the products of the selective hydrolysis of membrane phospholipids, eicosanoids, prostaglandins, thromboxanes, leukotrienes, aspirin, nonsteroidal anti-inflammatory agents, analgesic-antipyretic agents, agents that inhibit the synthesis of prostaglandins and thromboxanes, selective inhibitors of the inducible cyclooxygenase, selective inhibitors of the inducible cyclooxygenase-2, autacoids, paracrine hormones, somatostatin, gastrin, cytokines that mediate interactions involved in humoral and cellular immune responses, lipid-derived autacoids, eicosanoids, β-adrenergic agonists, ipratropium, glucocorticoids, methylxanthines, sodium channel blockers, opioid receptor agonists, calcium channel blockers, membrane stabilizers and leukotriene inhibitors.

Additional therapeutic agents contemplated herein include diuretics, vasopressin, agents affecting the renal conservation of water, rennin, angiotensin, agents useful in the treatment of myocardial ischemia, anti-hypertensive agents, angiotensin converting enzyme inhibitors, β-adrenergic receptor antagonists, agents for the treatment of hypercholesterolemia, and agents for the treatment of dyslipidemia.

Other therapeutic agents contemplated include drugs used for control of gastric acidity, agents for the treatment of peptic ulcers, agents for the treatment of gastroesophageal reflux disease, prokinetic agents, antiemetics, agents used in irritable bowel syndrome, agents used for diarrhea, agents used for constipation, agents used for inflammatory bowel disease, agents used for biliary disease, agents used for pancreatic disease. Therapeutic agents used to treat protozoan infections, drugs used to treat Malaria, Amebiasis, Giardiasis, Trichomoniasis, Trypanosomiasis, and/or Leishmaniasis, and/or drugs used in the chemotherapy of helminthiasis Other therapeutic agents include antimicrobial agents, sulfonamides, trimethoprim-sulfamethoxazole quinolones, and agents for urinary tract infections, penicillins, cephalosporins, and other, beta-Lactam antibiotics, an agent comprising an aminoglycoside, protein synthesis inhibitors, drugs used in the chemotherapy of tuberculosis, mycobacterium avium complex disease, and leprosy, antifungal agents, antiviral agents including nonretroviral agents and antiretroviral agents.

Examples of therapeutic antibodies that can be combined with a subject compound include but are not limited to anti-receptor tyrosine kinase antibodies (cetuximab, panitumumab, trastuzumab), anti CD20 antibodies (rituximab, tositumomab), and other antibodies such as alemtuzumab, bevacizumab, and gemtuzumab.

Moreover, therapeutic agents used for immunomodulation, such as immunomodulators, immunosuppressive agents, tolerogens, and immunostimulants are contemplated by the methods herein. In addition, therapeutic agents acting on the blood and the blood-forming organs, hematopoietic agents, growth factors, minerals, and vitamins, anticoagulant, thrombolytic, and antiplatelet drugs.

Further therapeutic agents that can be combined with a subject compound may be found in Goodman and Gilman's The Pharmacological Basis of Therapeutics, Tenth Edition, edited by Hardman, Limbird and Gilman, or the Physician's Desk Reference, both of which are incorporated herein by reference in their entirety.

The compounds described herein can be used in combination with the agents disclosed herein or other suitable agents, depending on the condition being treated. Hence, in some embodiments the compounds of the invention will be co-administer with other agents as described above. When used in combination therapy, the compounds described herein may be administered with the second agent simultaneously or separately. This administration in combination can include simultaneous administration of the two agents in the same dosage form, simultaneous administration in separate dosage forms, and separate administration. That is, a compound described herein and any of the agents described above can be formulated together in the same dosage form and administered simultaneously. Alternatively, a compound of the present invention and any of the agents described above can be simultaneously administered, wherein both the agents are present in separate formulations. In another alternative, a compound of the present invention can be administered just followed by and any of the agents described above, or vice versa. In the separate administration protocol, a compound of the present invention and any of the agents described above may be administered a few minutes apart, or a few hours apart, or a few days apart.

### EXAMPLES

The examples and preparations provided below further illustrate and exemplify the compounds of the present invention and methods of preparing such compounds. It is to be understood that the scope of the present invention is not limited in any way by the scope of the following examples and preparations. In the following examples molecules with a single chiral center, unless otherwise noted, exist as a racemic mixture. Those molecules with two or more chiral centers, unless otherwise noted, exist as a racemic mixture of diastereomers. Single enantiomers/diastereomers may be obtained by methods known to those skilled in the art.

### Example 1

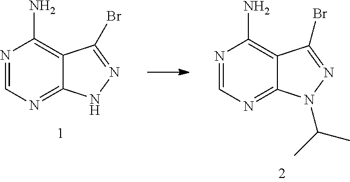

3-Bromo-1H-pyrazolo[3,4-d]pyrimidin-4-amine (1) (21.4 g, 0.1 mol) and potassium carbonate (27.64 g, 0.2 mol, 2 eq) were suspended in anhydrous DMF (110 mL) and stirred at 60° C. for 0.5 h. To this mixture, iso-propyl bromide (9.9 mL, 0.105 mol, 1.05 eq) was added at the same temperature. The resulting mixture was stirred at 60° C. for additional 2.5 h and then was allowed to cool to room temperature. The mixture was filtered, the cake was washed with small amount of isopropyl acetate and the filtrate was concentrated in vacuo. The residue was partitioned between water and isopropyl acetate (100 mL/400 mL). The aqueous layer was extracted with isopropyl acetate (100 mL×2). The combined organic layers were washed with brine (100 mL), dried over MgSO₄, filtered and rinsed with isopropyl acetate (50 mL×3). The filtrate was concentrated in vacuo to afford the crude product (23.4 g, 91.4% yield) as a yellow solid. The product obtained was suspended in methanol (25 mL) and stirred for 1 h. The solid was collected by filtration, rinsed with methanol (4 mL), and dried in vacuo to afford the desired product 2 (18.8 g, 73.4% yield) as a yellow solid. ¹H NMR (400 MHz, DMSO-d₆): δ 8.23 (s, 1H, pyrimidine), 5.00 (m, 1H, iPr), 1.44 (d, J=6.8 Hz, 6H, iPr); ¹³C NMR (100 MHz, DMSO-d₆): δ 157.3, 156.4, 152.9, 116.6, 99.4, 48.7, 21.6.

### Example 2

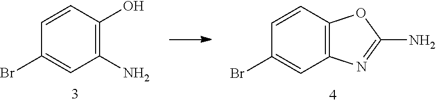 [SMILES: Nc1cc(Br)ccc1O ; Nc1nc2cc(Br)ccc2o1]

To a stirred solution of 2-amino-4-bromophenol (3) (59.6 g, 0.317 mol) in methanol (600 mL) at room temperature, solid bromine cyanide (40.3 g, 0.38 mol, 1.2 eq) was added carefully in portions and the resulting mixture was stirred at 35° C. for 6 h. (Note: the bromine cyanide is very toxic, the reagent and reaction should be handled carefully in the fume hood). The reaction mixture was quenched by addition of saturated aqueous Na₂CO₃ solution and the pH value was adjusted to 7-8. The mixture was then concentrated in vacuo to remove the methanol. The residue was dissolved in ethyl acetate (600 mL), washed with water (100 mL×2) and brine (100 mL), dried over MgSO₄ and filtered. The filtrate was concentrated in vacuo to afford the desired product 4 (65.2 g, 96.5% yield) as a yellow solid. ¹H NMR (400 MHz, DMSO-d₆): δ 7.62 (s, 2H), 7.37 (d, J=1.9 Hz, 1H), 7.30 (d, J=8.3 Hz, 1H), 7.11 (dd, J=8.3, 2.1 Hz, 1H); ¹³C NMR (100 MHz, DMSO-d₆): δ 163.7, 147.1, 145.7, 122.2, 117.7, 115.4, 110.0.

### Example 3

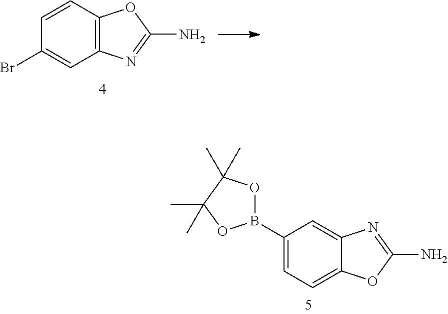 [SMILES: Nc1nc2cc(Br)ccc2o1 ; CC1(C)OB(c2ccc3oc(N)nc3c2)OC1(C)C]

5-Bromobenzo[d]oxazol-2-amine (4) (15.0 g, 70.4 mmol) and bis(pinacolato)diboron (21.5 g, 84.5 mmol, 1.2 eq) were dissolved in 1,4-dioxane (150 mL). To this mixture, PdCl₂(dppf) (5.17 g, 6.3 mmol, 0.09 eq) and potassium acetate (20.71 g, 211 mmol, 3 eq) were added sequentially. The resulting mixture was degassed and back-filled with argon three times and then refluxed at 110° C. for 2 h with stirring. The mixture was allowed to cool to room temperature, filtered, and the cake was rinsed with ethyl acetate (30 mL×2). The filtrate was mixed with silica gel (50 g) and then concentrated in vacuo. The residue was loaded onto a plug of silica gel (60 g), eluted with ethyl acetate/heptane (1:1, 1000 mL). The filtrate was concentrated in vacuo, the residue was suspended in heptane (50 mL) and refluxed for 30 min with stirring. The suspension was cooled to room temperature, and then the solid was collected by filtration and rinsed with small amount of heptane to afford the desired product 5 (15.66 g, 85.3% yield) as a yellow solid. ¹H NMR (400 MHz, DMSO-d₆): δ 7.47 (s, 1H), 7.43 (s, 2H), 7.34 (s, 2H), 1.30 (s, 12H).

### Example 3a

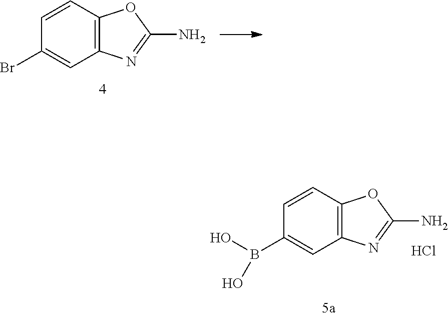 [SMILES: Nc1nc2cc(Br)ccc2o1 ; Nc1nc2cc(B(O)O)ccc2o1 ; Cl]

Compound 4 (6.9 kg) was charged to a 100 liter glass reactor at room temperature followed by the addition of 9.9 kg of bis(pinacolato)diboron and 69.0 kg of 1,4-dioxane. The reaction mixture was stirred under argon atmosphere, then 2.4 kg of 1,1′-bis(diphenylphosphino)ferrocene]dichloropalladium(II) (complex with dicloromethane 1:1) and 9.5 kg of potassium acetate were added. The reaction mixture was heated for 3.5 hours at 100° C. under argon atmosphere until the in-process HPLC test showed the reaction was complete. After cooling the reaction mixture to 25° C., it was loaded onto a plug of 20.6 kg of silica gel and filtered. The filter cake was washed with 230.0 kg of ethyl acetate. The combined filtrates were distilled under vacuum to approximately 15 liters. A mixture of 38.8 kg of concentrated hydrochloric acid and 32.5 kg of water were added. The reaction mixture was heated for 2.5 hours at 80° C. until the in-process HPLC test showed reaction was complete. The reaction mixture was cooled to 20° C. and filtered. The solid product 5a was washed with a mixture of 3.9 kg of concentrated hydrochloric acid and 36.0 kg of water followed by 44.2 kg of ethyl acetate and then dried at 50° C. for 90 hours under vacuum with a slight nitrogen bleed.

### Example 4

### Synthesis of 5-(4-amino-1-isopropyl-1H-pyrazolo[3,4-d]pyrimidin-3-yl)benzo[d]oxazol-2-Amine (Formula I)

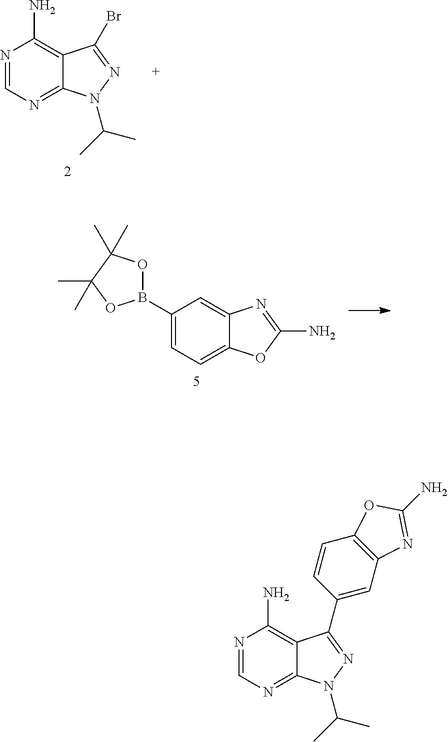

3-Bromo-1-isopropyl-1H-pyrazolo[3,4-d]pyrimidin-4-amine (2) (20 g, 78.1 mmol) and 5-(4,4,5,5-tetramethyl-1,3,2-dioxaborolan-2-yl)benzo[d]oxazol-2-amine (5) (26.4 g, 102 mmol, 1.3 eq) were dissolved in a mixture of 1,4-dioxane and water (300 mL/100 mL). To this mixture Pd(PPh₃)₄ (7.21 g, 6.25 mmol, 0.08 eq) and sodium carbonate (41.4 g, 391 mmol, 5 eq) were added sequentially. The resulting mixture was degassed and back-filled with argon three times and then refluxed at 110° C. for 3 h with stirring. The mixture was allowed to cool to room temperature, filtered and the cake was washed with ethyl acetate (50 mL×2). The combined filtrate was concentrated in vacuo. The residue was suspended in a mixture of water and ethyl acetate (500 mL/100 mL) and stirred for 30 min. The solid was collected by filtration, rinsed with water (50 mL) and ethyl acetate (100 mL). The crude product thus obtained was suspended in ethyl acetate (100 mL) and stirred for 30 min. The solid was collected by filtration, rinsed with ethyl acetate (50 mL), and dried in vacuo to afford the crude product of Formula I (20 g, 83% yield). The above obtained product (20 g) was dissolved in refluxing methanol (1600 mL), and activated charcoal (6 g, 30% W/W) was added. The mixture was refluxed for 30 min, and then the hot mixture was filtered through a Buchner funnel. The cake was washed with hot methanol (100 mL×3). The combined filtrates were concentrated. The solid was slurried in ethyl acetate (300 mL), and the suspension was stirred at room temperature for 30 min. The solid was collected by filtration, washed with ethyl acetate (50 mL×2), and dried in vacuo to afford the desired product of Formula I as polymorph Form A (16.27 g, 67.3% yield). m.p.: 273.67° C. (Onset Temperature); ¹H NMR (400 MHz, DMSO-d₆): δ 8.26 (s, 1H, pyrimidine), 7.56 (s, 2H, oxazol-2-amine), 7.48 (d, J=8.1 Hz, 1H, Ph), 7.45 (d, J=1.4 Hz, 1H, Ph), 7.27 (dd, J=8.1, 1.6 Hz, 1H, Ph), 5.08 (m, 1H, iPr) and 1.52 (d, J=6.7 Hz, 6H, iPr); ¹³C NMR (100 MHz, DMSO-d₆): δ 163.4, 158.1, 155.4, 153.2, 148.3, 144.4, 143.7, 128.8, 120.5, 115.0, 108.8, 97.5, 48.0 and 21.8; analysis (% calcd, % found for C₁₅H₁₅N₇O): C, (58.24, 58.04); H, (4.87, 4.83); N, (31.70, 31.49). greater than 99% purity based on LC-MS analysis.

### Example 4a

### Synthesis of 5-(4-amino-1-isopropyl-1H-pyrazolo[3,4-d]pyrimidin-3-yl)benzo[d]oxazol-2-Amine (Formula I)

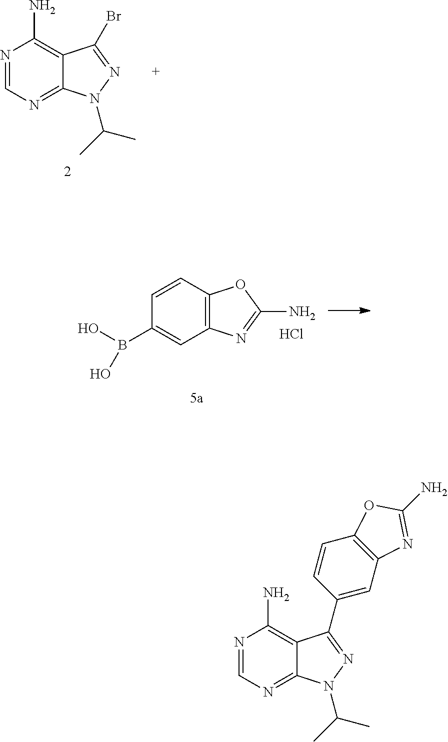

To a 100 liter glass reactor at room temperature was charged compound 2 (2.7 kg) and compound 5a (2.8 kg). The reaction mixture was stirred under nitrogen atmosphere and then 43.4 kg of 1,4-dioxane and 14.0 kg of water were cadded. The reaction mixture was stirred under argon atmosphere followed by the addition of 1.0 kg of tetrakis(triphenylphosphine)palladium(0) and 5.7 kg of sodium carbonate. The reaction mixture was heated at reflux (88° C.) for 7.5 hours under argon atmosphere until in-process HPLC showed completion of the reaction. After cooling room temperature, the reaction mixture was distilled under vacuum to approximately 10 L. To this mixture were added 60.0 kg of water and 11.0 kg of ethyl acetate. The mixture was stirred at 22° C. for 1 hour and then filtered. The wet cake was transferred to a 100 liter glass reactor, mixed with 60.0 kg of water and 11.0 kg of ethyl acetate, and stirred at 22° C. for 30 minutes. The mixture was filtered. The wet cake was washed with 8.0 kg of water and 8.5 kg of ethyl acetate. After washing, the wet cake was transfer to a 100 liter glass reactor, mixed with 12.6 kg of ethyl acetate and stirred at 22° C. for 30 minutes. The mixture was again filtered and washed with 5.7 kg of ethyl acetate. After drying the crude product at 54° C. under vacuum with a slight nitrogen bleed, it (2.36 kg) was charged to a 200 gal GLCS Still. The still was purge with nitrogen and 200.0 kg of methanol was added. The mixture was heated to 60° C. To this mixture was added a suspension of 1.0 kg of activated carbon in 14.5 kg of methanol. The resulting mixture was heated for 1 hour at 60° C. The hot mixture was filtered through a preheated (60° C.) glass Nutsche filter. The cake was wash with 70.0 kg of hot methanol. The combined filtrates were distilled under vacuum to approximately 10 L. 28.4 kg of ethyl acetate were added. The mixture was stir at 25° C. for 30 minutes and filtered. The wet cake was washed with 12.7 kg of ethyl acetate. The desired product of Formula I was dried at 50° C. under vacuum with a slight nitrogen bleed until the Loss on Drying is not more than 1.0%.

### Example 5

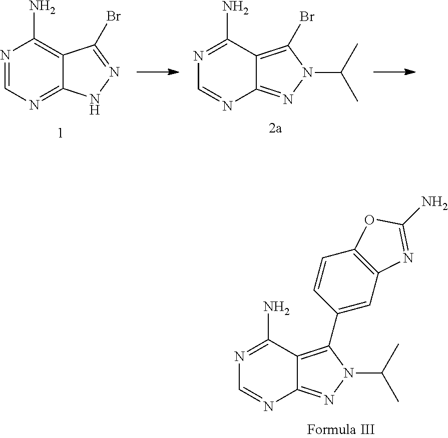 [SMILES: Nc1ncnc2[nH]nc(Br)c12 ; CC(C)n1nc2ncnc(N)c2c1Br ; CC(C)n1nc2ncnc(N)c2c1-c1ccc2oc(N)nc2c1]

3-Bromo-1H-pyrazolo[3,4-d]pyrimidin-4-amine (1) (21.4 g, 0.1 mol) and potassium carbonate (27.64 g, 0.2 mol, 2 eq) were suspended in anhydrous DMF (210 mL) and stirred at 80° C. for 0.5 h. To this mixture, iso-propyl bromide (9.9 mL, 0.105 mol, 1.05 eq) was added at the same temperature. The resulting mixture was stirred at 80° C. for additional 2.5 h and then was allowed to cool to room temperature. The mixture was concentrated in vacuo and the residue was purified by flash column chromatography on silica gel (MeOH/DCM: 1:80 to 1:10) to afford the desired product 3-bromo-2-isopropyl-2H-pyrazolo[3,4-d]pyrimidin-4-amine (2a) (800 mg, 3.1%) as a solid.

3-bromo-2-isopropyl-2H-pyrazolo[3,4-d]pyrimidin-4-amine 2a (500 mg, 1.95 mmol, 1.0 eq), 5-(4,4,5,5-tetramethyl-1,3,2-dioxaborolan-2-yl)benzo[d]oxazol-2-amine (1 g, 3.9 mmol, 2.0 eq), Pd(OAc)₂ (131 mg, 0.59 mmol, 0.3 eq), PPh₃ (308 mg, 1.17 mmol, 0.6 eq) and Na₂CO₃ (1.03 g, 9.75 mmol, 5.0 eq) were dissolved in DMF/EtOH/H₂O (30 mL/10 mL/10 mL). The resulting mixture was degassed and back-filled with argon three times, and then stirred at 80-90° C. under an argon atmosphere for 1.5 h. The reaction was complete based on TLC analysis. The mixture was concentrated in vacuo and the residue was purified by flash column chromatography on silica gel (MeOH/DCM 1:100 to 1:10) to afford the desired product 5-(4-amino-2-isopropyl-2H-pyrazolo[3,4-d]pyrimidin-3-yl)benzo[d]oxazol-2-amine (Formula III) (300 mg, 52%) as a solid.

### Example 6

The X-Ray Powder Diffraction (XRPD) patterns of Form A was collected on a Siemens D5000 diffractometer using Cu Kα radiation (40 kV, 40 mA), 0-0 goniometer, divergence of V20 and receiving slits, a graphite secondary monochromator and a scintillation counter. The instrument is performance checked using a certified Corundum standard (NIST 1976). The software used for data collection was Diffrac Plus XRD Commander v2.3.1 and the data were analysed and presented using Diffrac Plus EVA v 11.0.0.2 or v 13.0.0.2.

The sample was prepared as flat plate specimens using powder as received. Approximately 35 mg of the sample was gently packed into a cavity cut into polished, zero-background (510) silicon wafer. The sample was rotated in its own plane during analysis. The details of the data collection are:
Angular range: 2 to 42·2θ
Step size: 0.05·2θ
Collection time: 4 s·step⁻¹

The XRPD patterns of other forms were collected on a Bruker AXS C2 GADDS diffractometer using Cu Kα radiation (40 kV, 40 mA), automated XYZ stage, laser video microscope for auto-sample positioning and a HiStar 2-dimensional area detector. X-ray optics consists of a single Gael multilayer mirror coupled with a pinhole collimator of 0.3 mm.

The beam divergence, i.e. the effective size of the X-ray beam on the sample, was approximately 4 mm. A θ-θ continuous scan mode was employed with a sample—detector distance of 20 cm which gives an effective 2θ range of 3.2°-29.7°. Typically the sample would be exposed to the X-ray beam for 120 seconds. The software used for data collection was GADDS for WNT 4.1.16 and the data were analysed and presented using Diffrac Plus EVA v 9.0.0.2 or v 13.0.0.2.

Samples were prepared as flat plate specimens using powder as received without grinding. Approximately 1-2 mg of the sample was lightly pressed on a silicon wafer to obtain a flat surface.

### Example 7

### Studies on Form A

X-Ray Powder Diffraction

The high-resolution XRPD diffractogram of Form A is shown on FIG. 1.

Stability Testing at 40° C./75% RH

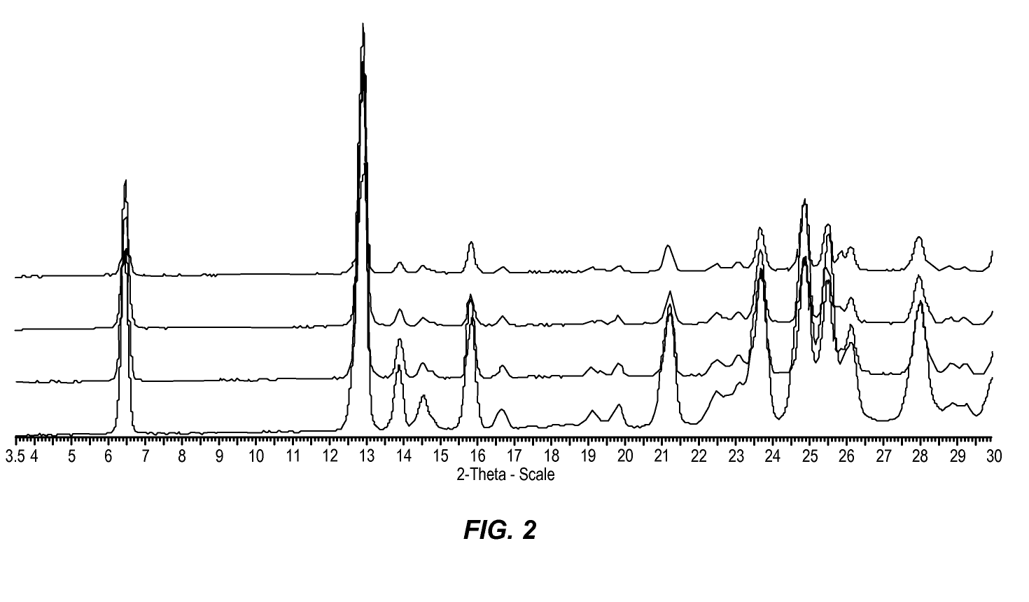

A sample of J00439 was stored at 40° C./75% RH. Reanalysis by XRPD after 7 days, then after 21 days and after 35 days showed it was still pattern A (FIG. 2). Thus this solid form (which will be referred to as Form A) was stable to accelerated stability testing conditions. Note that some variations in XRPD peak intensity were observed over time.

Differential Scanning Calorimetry (DSC) and Thermo-Gravimetric Analysis (TGA)

The TGA trace of Form A (FIG. 3) showed only a small weight loss between 25° C. and 250° C. The 9.7% weight loss between 250° C. and 350° C. is likely to be due to some degradation. The DSC trace of Form A (FIG. 4) showed a sharp melting endotherm with onset at 274° C.

Gravimetric Vapour Sorption (GVS)

Data from the GVS experiment of Form A showed very little weight change over the whole experiment (0.1%). There was no hysteresis. The material is non-hygroscopic. The kinetic plot (FIG. 5) showed quick equilibration at every RH. The material recovered after the GVS experiment was reanalyzed by XRPD and was still pattern A. This form was therefore stable to the GVS conditions.

Water Determination by Karl Fischer

Water content of J00439 was 2.4% by Karl Fischer titration.

### Example 8

### Polymorph Screening

Experiments were carried out to screen for different polymorphs. The results are summarized in Tables 1-3. Specifically, in Table 1, a solution of Form A was treated with an anti-solvent. In Table 2, solutions of Form A in a variety of single-solvents were cooled to 5° C. at 0.5° C./min. In Table 3, solutions of Form A in a variety of water/organic solvent mixture were cooled to 5° C. at 0.5° C./min. Solids obtained were further analyzed by X-Ray Powder Diffraction (XRPD) method described in Example 6. All the experiments in Table 2 and 3 were carried out on 20 mg scale.

TABLE 1
Polymorph screening with anti-solvent

| | Form | | Volume | | | XRPD of solid |
|---|---|---|---|---|---|---|
| Entry | A (mg) | Solvent | (Vols) | Result | Comments | filtered out |
| 1 | 12.19 | DMSO | 17 | Complete dissolution, heated | Poured into 1 ml H₂O. Immediate | partially |
| | | | | with heatgun to dissolve | precipitation | crystalline, |
| | | | | possible seeds | | pattern A |
| 2 | 12.70 | DMSO | 17 | Complete dissolution, heated | Quickly added −500 μl of meOH. Solution | crystalline, |
| | | | | with heatgun to dissolve | turned cloudy, then cleared up again. | pattern A |
| | | | | possible seeds | Crystallisation took place after a while | |
| 3 | 10.31 | DMF | 28 | Dissolved, filtered to remove | lost on filtration | n/a |
| | | | | possible small seeds | | |
| 4 | 11.28 | NMP | 40 | Dissolved, filtered to remove | Added large amount of H₂O, stayed as a | crystalline, |
| | | | | possible small seeds | clear solution at first, then solid started to | pattern A |
| | | | | | crystallize | |
| 5 | 10.52 | DMA | 44 | Dissolved, filtered toremove | Added large amount of H₂O, stayed as a | crystalline, |
| | | | | possible small seeds | clear solution at first, then solid started to | pattern A |
| | | | | | crystallize | |
| 6 | 11.21 | dioxane | >150 | Incomplete dissolution, | Added large amount of H₂O, but stayed as a | Large crystals, |
| | | | | filtered | clear solution | kept for SCXRD |
| 7 | 11.95 | DMF | 28 | Dissolved, filtered to remove | Added large amount of H₂O, stayed as a | partially |
| | | | | possible small seeds | clear solution at first, then solid started to | crystalline, |
| | | | | | crystallize | pattern A |

TABLE 2
Polymorph screening with single-solvents

| | | | | | | XRPD pattern |
|---|---|---|---|---|---|---|
| | | | | | XRPD pattern | of solid after |
| | | Volume | Temp. | | of solid | cooling of |
| Entry | Solvent | (vols) | (° C.) | Observations while hot | filtered hot | solution |
| 8 | DMSO | 5 | 120 | full dissolution, cooled down | | partially crystalline |
| | | | | | | A |
| 9 | DMF | 5 | 120 | full dosslution, cooled down | | A + B + X? |
| 10 | DMA | 5 | 120 | full dosslution, cooled down | | B |
| 11 | NMP | 5 | 120 | full dosslution, cooled down | | still a clear |
| | | | | | | solution (yellow) |
| 12 | MIBK | 50 | 100 | suspension, hot filtration, then | A | too little solid to |
| | | | | liquors cooled down | | analyse |
| 13 | Toluene | 50 | 100 | suspension, hot filtration, then | A | still a clear |
| | | | | liquors cooled down | | solution |
| 14 | water | 50 | 95 | suspension, hot filtration, then | A | |
| | | | | liquors cooled down | | |
| 15 | acetic | 10 | 95 | full dissolution, cooled down | | still a clear |
| | acid | | | | | solution |
| 16 | dioxane | 40 | 95 | full dissolution, cooled down | | A |
| 17 | IPA | 50 | 75 | suspension, hot filtration, then | A | A |
| | | | | liquors cooled down | | |
| 18 | EtOH | 50 | 75 | suspension, hot filtration, then | A | A |
| | | | | liquors cooled down | | |
| 19 | MeCN | 50 | 75 | suspension, hot filtration, then | A | partially crystalline |
| | | | | liquors cooled down | | A |
| 20 | Me—THF | 50 | 75 | suspension, hot filtration, then | A | too little solid to |
| | | | | liquors cooled down | | analyse |
| 21 | THF | 50 | 65 | fine suspension, hot filtration then | A | A |
| | | | | liquors cooled down | | |

TABLE 3
Polymorph screening with water/organic solvent mixtures

| | | | Water | | | | | XRPD |
|---|---|---|---|---|---|---|---|---|
| | | Volume | volume | Water | Temp. | Observations while hot, | Observation | pattern of |
| Entry | Solvent | (vols) | (vols) | % v/v | (° C.) | after addition of H₂O | after cooling | solid |
| 22 | DMSO | 5 | 5 | 50 | 95 | immediate | slurry | Mostly |
| | | | | | | precipitation | | amorphous, |
| | | | | | | | | traces of A |
| 23 | DMSO | 15 | 10 | 40 | 95 | precipitation | suspension | A |
| 24 | DMF | 15 | 5 | 25 | 95 | stays clear | thick suspension | A |
| 25 | DMF | 15 | 10 | 40 | 95 | clear then starts to | fine suspension + solid | A |
| | | | | | | precipitate | stocking to bottom | |
| 26 | DMA | 15 | 5 | 25 | 95 | stays clear | clear - adding further 20 | A |
| | | | | | | | vols of H₂O gives | |
| | | | | | | | suspension | |
| 27 | DMA | 15 | 10 | 40 | 95 | stays clear | clear - adding further 20 | A |
| | | | | | | | vols of H₂O leads to | |
| | | | | | | | precipitation of sticky solid | |
| 28 | NMP | 5 | 5 | 50 | 95 | clear then starts to | thick slurry | A |
| | | | | | | precipitate | | |
| 29 | NMP | 5 | 10 | 66.7 | 95 | clear then starts to | suspension | A |
| | | | | | | precipitate | | |
| 30 | acetic | 5 | 10 | 66.7 | 95 | clear then starts to | small amount of solid | D (+some |
| | acid | | | | | precipitate | sticking to sides - added | A?) |
| | | | | | | | 20 vols H₂O [symbol] more | |
| | | | | | | | solid came sout | |
| 31 | acetic | 5 | 20 | 80 | 95 | clear then starts to | solution + solid sticking to | A |
| | acid | | | | | precipitate | sides and bottom of vial | |
| 32 | dioxane | 50 | 10 | 16.7 | 95 | stays clear | clear - adding further 20 | C |
| | | | | | | | vols of H₂O gives | |
| | | | | | | | suspension | |
| 33 | dioxane | 50 | 20 | 28.6 | 95 | stays clear | clear - adding further | C |
| | | | | | | | 20 vols of H₂O gives | |
| | | | | | | | suspension | |
| 34 | IPA | 25 | 10 | 28.6 | 75 | suspension before | suspension | A |
| | | | | | | addition of H₂O, | | |
| | | | | | | dissoluves on addition | | |
| 35 | EtOH | 25 | 10 | 28.6 | 75 | suspension before | suspension | A |
| | | | | | | addition of H₂O, mostly | | |
| | | | | | | dissoluves on addition | | |
| 36 | MeCN | 25 | 10 | 28.6 | 75 | suspension (before and | suspension | A |
| | | | | | | after H₂O addition) | | |
| 37 | THF | 25 | 10 | 28.6 | 65 | fine suspension (before | suspension | C |
| | | | | | | and after H₂O addition) | | |

### Example 9

### Scale-Up Experiments for Polymorph Screening and Characterizations

The experiments that had led to solids with different XRPD patterns were repeated on an 80 to 100 mg scale. Details are shown on Table 4. All solids were filtered, air-dried for a few minutes on the filter, and then dried for 1 h in a vacuum oven (25° C.) before analysis. Entry 38 was the repeat of the small-scale experiment in Table 2 (entry 9), which had given an apparent mixture of patterns. But on this larger scale, cooling of the DMF solution produced a new Form E. Also, entry 40, in AcOH/water, which was expected to produce Form D, gave the Form A of the starting material on this occasion. The other experiments led to the expected Forms B and C.

TABLE 4
Scale-up experiments

| | | Mass of | | | | | | |
|---|---|---|---|---|---|---|---|---|
| | XRPD | SM | | | Water | | | XRPD |
| | pattern | J00439 | | Volume | volume | | Observation | pattern of |
| Entry | expected | (mg) | Solvent | (vols) | (vols) | Conditions | after cooling | solid |
| 38 | A + B + X? | 80.8 | DMF | 5 | | Dissolved at 120° C., then | suspension | E |
| | | | | | | cooled down to 5° C. at | | |
| | | | | | | 0.5° C./min | | |
| 39 | B | 79.9 | DMA | 4 | | Dissolved at 120° C., then | suspension | B |
| | | | | | | cooled down to 5° C. at | | |
| | | | | | | 0.5° C./min | | |
| 40 | D | 105 | AcOH | 5 | 30 | Dissolved at 95° C., added | suspension | A |
| | | | | | | H₂O, then cooled down to | | |
| | | | | | | 20° C. at 0.5° C./min | | |
| 41 | C | 94 | Dioxane | 50 | 40 | Dissolved at 95° C., added | clear-added 10 | C |
| | | | | | | H₂O, then cooled down to | vols H₂O [symbol] | |
| | | | | | | 20° C. at 0.5° C./min | precipitation | |
| 42 | C | 93 | THF | 25 | 10 | Dissolved at 65° C., added | suspension | C |
| | | | | | | H₂O, then cooled down to | | |
| | | | | | | 20° C. at 0.5° C./min | | |

Besides XRPD analysis, the solids obtained were subjected to further analyses. The result is summarized in Table 5.

TABLE 5
Summary of characterization of scale-up solids

| | | | TGA weight loss | | | |
|---|---|---|---|---|---|---|
| | | | (excluding decomp. | | | KF |
| | Crystallisation | XRPD | weight loss after | | | Water |
| Entry | solvent | pattern | 250° C.) | DST (T onset, area) | ¹H NMR | content |
| 38 | DMF | E | Sharp 28.1% loss | 3 low temperature endotherms: small | 1.5 mol eq | 16.0% |
| | | | between 30° C. | 49° C., 2 J/g; 61° C., 67 J/g, 76° C., 110 | DMF | |
| | | | and 85° C. | J/g, then sharp melt: 271° C., 118 J/g | | |
| 39 | DMA | B | 1.0% loss between | 3 low temperature endotherms: 73° C. | 1.4 mol eq | 7.9% |
| | | | 60° C. and 90° C., | 12 J/g; 85° C., 16 J/g, 118° C., 136 J/g, | DMA | |
| | | | 19.4% between 90° C. | then sharp melt: 272° C., 115 J/g | | |
| | | | and 120° C. | | | |
| 40 | AcOH/H₂O | A | 0.4% loss between | sharp endotherm 271° C., 146 J/g | 0.056 mol | not done |
| | | | 30° C. and 170° C. | | eq residual | |
| | | | | | AcOH | |
| 41 | Dioxane/H₂O | C | 16.3% loss between | 91° C., 197 J/g, with shoulder on onset, | 0.48 mol eq | 12.8% |
| | | | 40° C. and 120° C. | then sharp melt: 273° C., 142 J/G | dioxane | |
| 42 | THF/H₂O | C | 14.5% loss between | 86° C., 198 J/g, with sholder on onset | 0.43 mol eq | 12.1% |
| | | | 50° C. and 110° C. | then sharp melt: 273° C., 146 J/g | THF | |

### Thermal Analysis

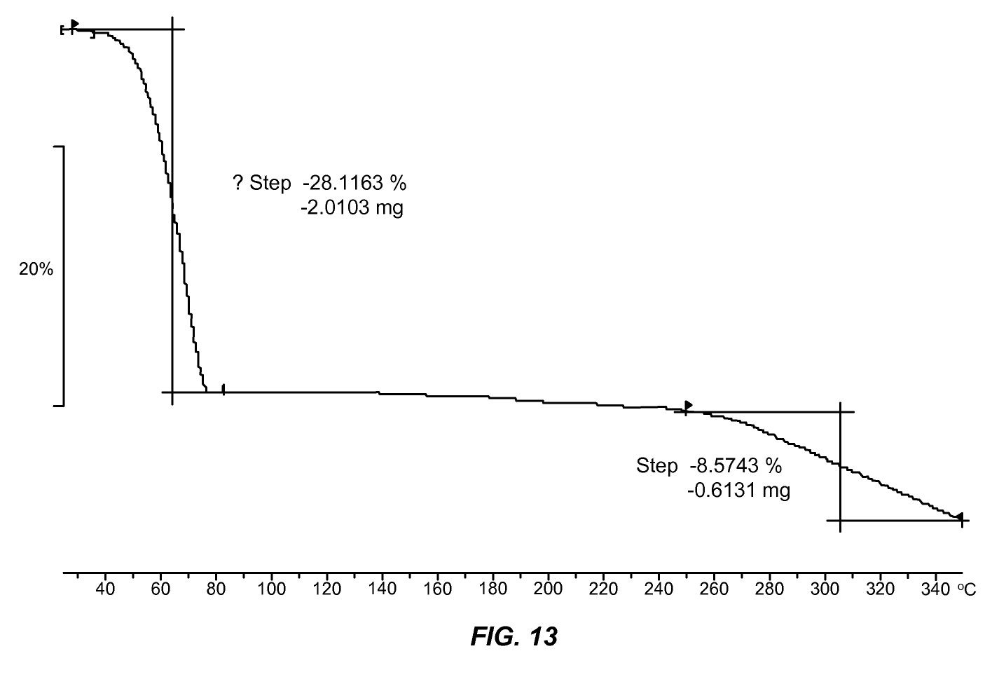

Form E solid (entry 38, Table 5) had a sharp step around 60° C. in the TGA (FIG. 13), losing 28% weight.

Multiple low temperature endotherms were observed in the DSC, followed by the sharp melt characteristic of Form A at 271° C.

Similar thermal behaviour was observed for both samples of Form C solids, with a TGA step around 80° C. and a low-temperature DSC endotherm (FIG. 9).

Form B solid (entry 39, Table 5) also exhibited a step in the TGA curve, although this was made up of two consecutive steps around 70° C. and 100° C. The DSC trace included a complex succession of low-temperature endotherms. It is possible that part of these low temperature losses corresponded to unbound residual solvent.

Sharp steps in the TGA are typical of desolvation of bound solvent, and DSC indicates that desolvation leads to Form A.

¹H NMR

As shown on Table 11, ¹H NMR indicated the presence of solvent in solids of Forms B, C, and E. It is noticeable that the amounts of dioxane or THF seen in Form C solids were very similar to that observed in the smaller scale experiments. The Form E solid contained 1.5 mol eq DMF, which would correspond to ca. 25.5% wt. This was a little lower than the 28% TGA step, so some water could also account for this weight loss. The Form B solid contained 1.4 mol eq DMA (28.3% wt). Some of the DMA is unbound residual solvent.

Karl Fischer Water Titration

Water contents of the solids by Karl Fischer titration are shown in Table 5. All solids contained significant amounts of water and therefore could be hydrates. In particular, Form C solids (entry 41 and 42, Table 5), had a similar level of water, which corresponds to ca. 2.5 mol eq (12.5% wt).

Drying of the Solids

A sample of each solid was dried in a vacuum oven at 40° C. overnight, then the samples were reanalysed by XRPD and ¹H NMR. Results are given in Table 6 below.

Form E desolvated to Form A on drying, while Form B appeared to have only partially desolvated, losing ca. 0.4 mol eq of DMA and exhibited a new XRPD pattern (Form F). Both Form C solids (entry 41 and 42, Table 6) were stable on drying and did not lose any solvent. This was particularly striking in the case of the relatively low-boiling THF, and pointed towards the organic solvent being bound in the structure.

TABLE 6
Drying of solids of Forms E, B, and C at 40° C. and reanalysis

| Sample ref | XRPD pattern | Solvent by ¹H NMR |
|---|---|---|
| before drying | before drying | before drying |
| 38 | E | 1.5 mol eq DMF |
| 39 | B | 1.4 mol eq DMA |
| 40 | C | 0.48 mol eq dioxane |
| 41 | C | 0.43 mol eq THF |
| 42 | A | 0.08 mol eq DMF |
| 43 | F | 0.94 mol eq DMA |
| 44 | C | 0.48 mol eq dioxane |
| 45 | C | 0.42 mol eq THF |

GVS Analysis

Form E lost 28% wt during the first sorption cycle (from 40 to 90% RH), then did not lose or gain any further weight during the desorption and second sorption cycles. Reanalysis of the solid after GVS by XRPD showed it had turned to Form A. This is consistent with desolvation of Form E to Form A in the GVS experiment.

Similarly, Form B desolvated to Form A, losing a total of 28% weight in various steps during the first sorption cycle.

Form C had a different behaviour in the GVS. It did not desolvate and was still Form C in the post-GVS XRPD. The sample was non-hygroscopic, with only 0.16% weight gain in the first sorption cycle. It did not dehydrate/desolvate on desorption either (0.38% loss between 90 and 0% RH). No hysteresis was observed, and the kinetic plot showed quick equilibration at every RH.

Stability Testing

Samples of entries 38, 39, 41, and 42 were stored at 40° C./75% RH. Reanalysis by XRPD after 7 days showed that Forms E and B had turned into Form A, while both Form C solids were still of Form C.

One of the Form C solids (entry 45, Table 6) was also reanalysed by ¹H NMR to check the level of solvent (THF) left after storing at high humidity. 0.37 mol eq of THF was measured by integration, which corresponded to a small decrease in the level of solvent. Again, this suggested that THF is quite firmly bound into the structure, as high humidity often helps in removing solvent (as observed for Forms E and B solvates).

In summary, various solvates of formula I were identified. Form B and Form E appeared to be respectively DMF and DMA solvates. They might be mixed hydrate/solvate (containing both water and organic solvent), as they had high level of water by Karl Fischer (but the water content alone could not explain the step observed in the TGA curve). Both the Form B and Form E readily desolvated to Form A in the GVS and under accelerated stability testing conditions (40° C./75% RH). Form E also turned into Form A on drying, while Form B appeared to partially desolvate to a new Form F.

Form C was stable to drying and high humidity conditions (both under GVS and stability testing). It appeared to be a hydrate with ca. 2.5 mol eq water. It also seemed to contain ca. 0.4 to 0.5 mol eq of THF or dioxane. It could therefore be a mixed hydrate/solvate. It is also possible that Form C was actually a trihydrate with partial occupation of the lattice by the organic solvent.

### Example 10

### Salt Screening Studies

Salts of Form A were formed with p-toluenesulfonic acid, ethane-1,2-disulfonic acid (EDSA), hydrochloric acid (HCl) (mono and bis), sulfuric acid, maleic acid, methanesulfonic acid (MSA), benzenesulfonic acid (BSA), ethanesulfonic acid (ESA), phosphoric acid, isethionic acid, and oxalic acid. Various salts were tested against various solvents for formation of crystalline solids, as shown in Table 7. Form A was observed to form crystalline mono-salts with hydrochloric acid and phosphoric acid and semicrystalline to crystalline bis-salts with sulfuric acid, hydrochloric acid, 1,2-ethane disulfuric acid, p-toluene sulfonic acid, methanesulfonic acid, ethanesulfonic acid, and maleic acid.

TABLE 7
Summary of results from salt screenings

| | Bis-salt small | | Bis-salt | | |
|---|---|---|---|---|---|
| | scale screen | Bis-sale scale-up | preparation from | Mono-sale | |
| Acid | from THF | preparation from THF | DMSO | screen | Stability at 40° C./75% RH |
| HCI | Partially | 2.1 mol eq choride by IC, | Same quality | Mono-chloride salt | Bis-salt changes pattern |
| | crystalline | complex thermal analysis, | bis-salt | (IC: 0.92 mol eq), | progressively, turning into |
| | | crystallinity not very high | | nice DSC, melt | mono-protonated salt, Mono- |
| | | | | at 237° C. | salt: stable for 7 days |
| H₂SO₄ | Crystalline | 2.0 mol eq sulfate by IC, 2 | No precipitation | Amorphous | Bis-salt: complex DSC after |
| | | endotherms in DSC, and | | | 10 days at 40° C./75% RH; |
| | | multiple loss in TGA | | | Mono-salt: crystallises |
| | | (possible partial decomp. | | | (mixture of XRPD patterns, |
| | | of the salt at 180° C.) | | | complex DSC) |
| 1,2-Ethane | Ball of gum that | | 0.98 mol eq | No significant | Stable for 7 days |
| disulfonic acid | crystallizes | | ethane disulfonate | precipitation | |
| | | | by NMR, 2 DSC | | |
| | | | endotherms | | |
| p-Toluene | Crystalline | 2.0 mol eq PTSA by | | No precipitation | Changes pattern after 3 days, |
| sulfonic acid | | NMR, good crystallinity, | | | but still bis-salt, higher |
| | | sharp melt at 246° C. | | | melting form |
| Methane | Partially | 2.0 mol eq mesylate by | | Crystalline bis- | Deliquescent |
| sulfonic acid | crystalline | NMR, good crystallinity, | | mesylate salt | |
| | | melt at 245° C. | | obtained | |
| Ethane sulfonic | Partially | 2.0 eq ethane sulfonate by | | No precipitation | Deliquescent |
| acid | crystalline | NMR, good crystallinity, | | | |
| | | melt at 181° C. | | | |
| Benzene | Cloudy solution | | No precipitation | | |
| sulfonic acid | | | | | |
| Oxalic acid | Crystalline but | | | | |
| | degredation | | | | |
| | suspected from | | | | |
| | NMR | | | | |
| Isethionic acid | Clear with some | | No precipitation | | |
| | oiling out | | | | |
| Maleic acid | Crystalline | 1.9 mol eq maleate by | | Crystalline bis- | Stable for 7 days |
| | | NMR, good crystallinity, | | maleate salt | |
| | | at 193° C.: melt or loss | | obtained | |
| | | of 1 eq maleate? | | | |
| Phosphoric | Crystalline | Mono-phosphate salt (1.0 | | Same mono- | Stable for 7 days |
| acid | | mol eq by IC), good | | phosphate salt | |
| | | crystallinity, sharp | | obtained | |
| | | melt at 225° C. | | | |

### Example 11

### Additional Solvate Screens

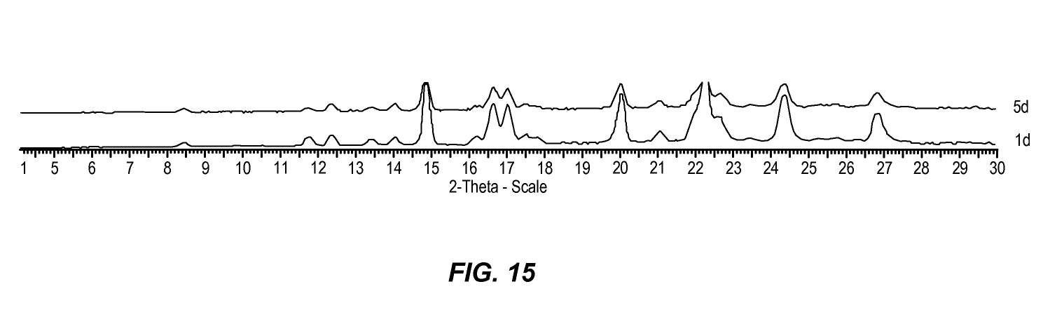

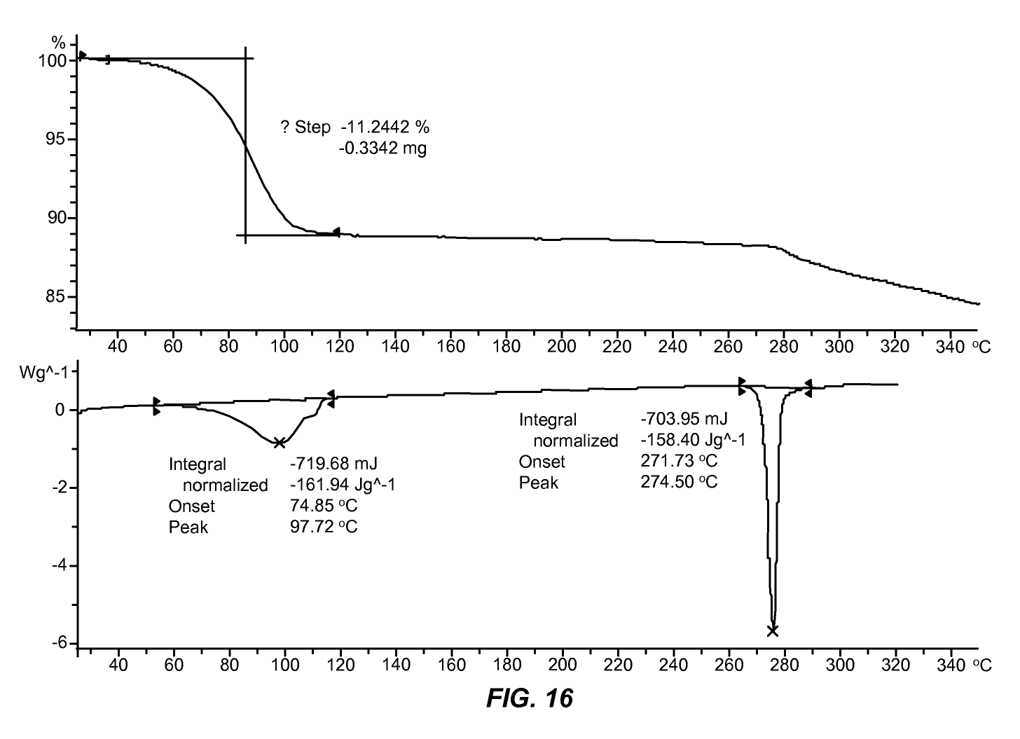

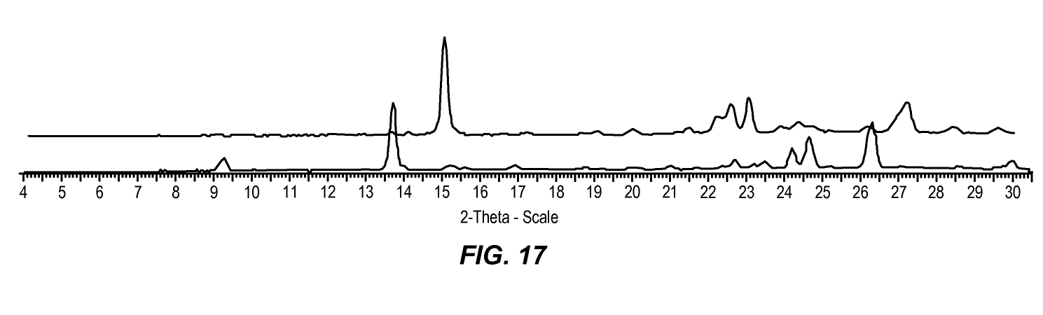

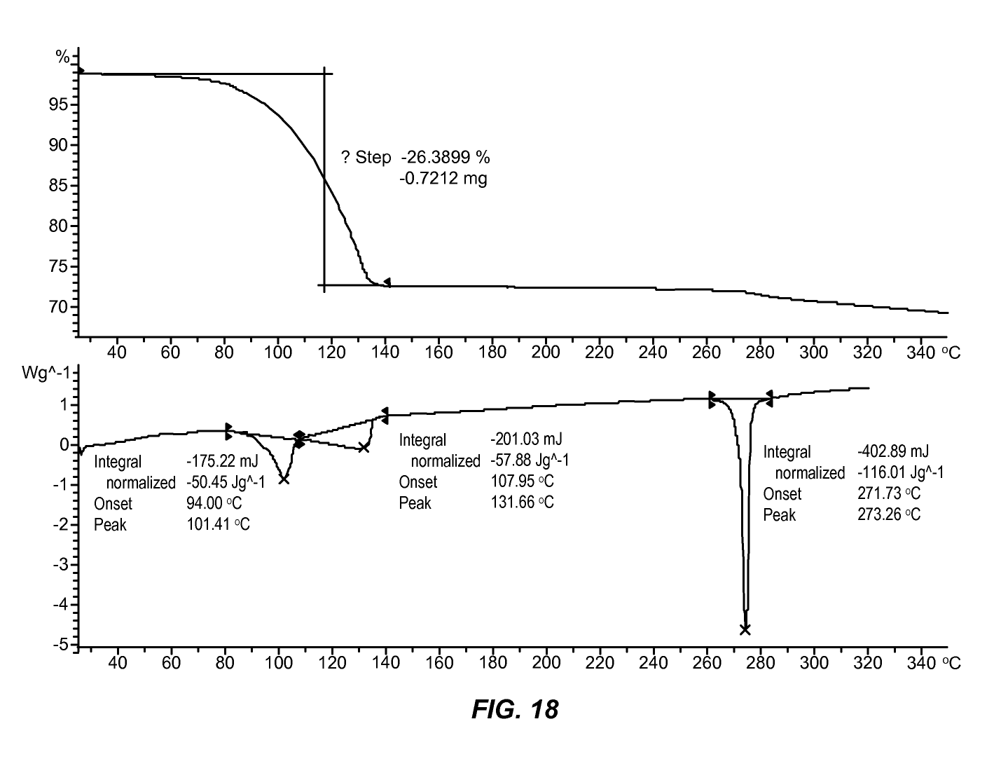

Approximately 20 mg of compound of Formula I (Form A) were weighed into vials, and 150 microliters of solvent system were added. The vials were agitated at 50° C., 5° C., or cycled between room temperature and 50° C. for 4 to 5 days. Solids were isolated by filtration and characterized by XRPD and thermal analysis. From acetone/5% water at 5° C., a hydrate was isolated (see FIGS. 15 and 16). The hydrate converted to Form A after vacuum drying, GVS analysis, 8 days storage at 40° C./75% relative humidity or 25° C./97% relative humidity, or heating to about 100° C. From dimethylacetamide (DMA) cycled between room temperature and 50° C. for 5 days, a solvate was isolated (see FIGS. 17 and 18). The DMA solvate converted to Form A after 8 days storage at 40° C./75% relative humidity or 25° C./97% relative humidity, or heating to about 100° C. Inclusion of 10% or more water with DMA prevented formation of DMA solvate.

While preferred embodiments of the present invention have been shown and described herein, it will be apparent to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1-11. (canceled)

12. A method of making polymorph Form A of the compound of Formula I: 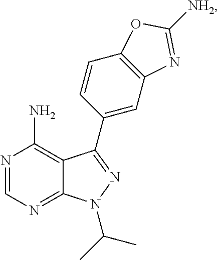 [SMILES: CC(C)n1nc(-c2ccc3oc(N)nc3c2)c2c(N)ncnc21]
said method comprising
(i) reacting compounds 2 and 5: 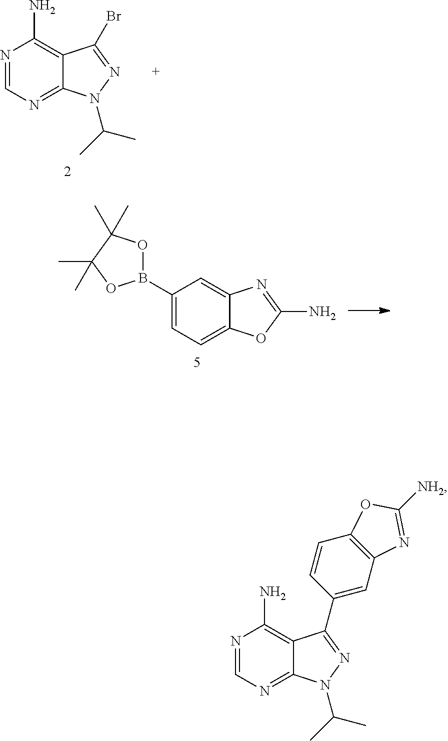
or reacting compounds 2 and 5a: 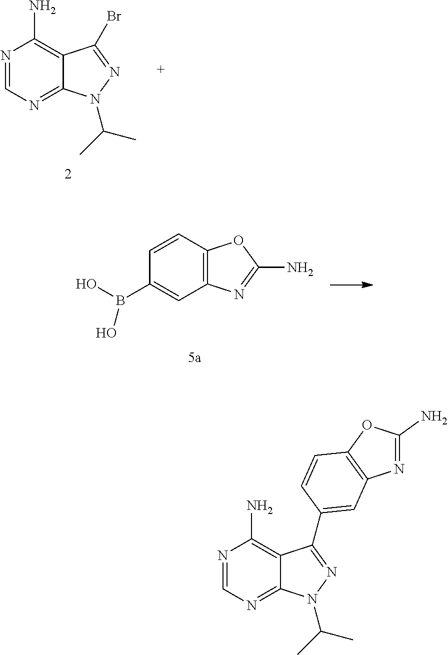 [SMILES: CC(C)n1nc(Br)c2c(N)ncnc21 ; Nc1nc2cc(B(O)O)ccc2o1 ; CC(C)n1nc(-c2ccc3oc(N)nc3c2)c2c(N)ncnc21]
to yield a compound of Formula I; and
(ii) isolating said compound of Formula I in polymorph Form A;
wherein step (ii) includes recrystallization of the compound of Formula I from either a mono-solvent system or from a multi-solvent system.

13. The method of claim 12, wherein said step (ii) includes recrystallization of the compound of Formula I from a mono-solvent system.

14. The method of claim 12, wherein said step (ii) includes recrystallization of the compound of Formula I from a multi-solvent system that does not contain dioxane or THF.

15. A method of making polymorph Form A of the compound of Formula I: 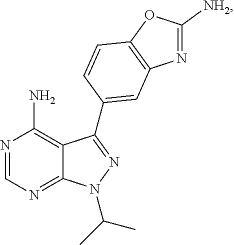 [SMILES: CC(C)n1nc(-c2ccc3oc(N)nc3c2)c2c(N)ncnc21]
said method comprising
(i) reacting compounds 2 and 5: 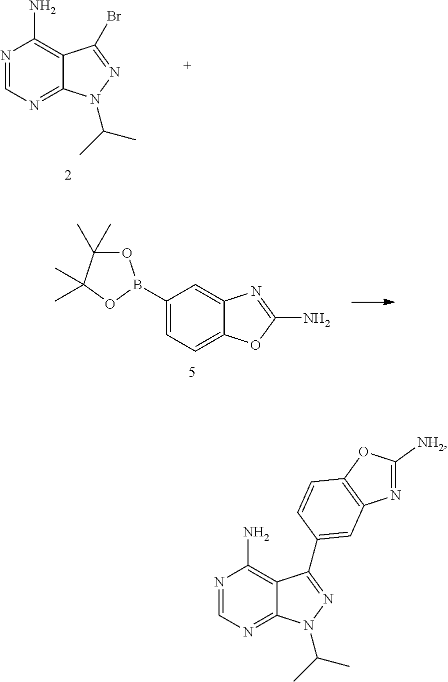 [SMILES: CC(C)n1nc(Br)c2c(N)ncnc21 ; CC1(C)OB(c2ccc3oc(N)nc3c2)OC1(C)C ; CC(C)n1nc(-c2ccc3oc(N)nc3c2)c2c(N)ncnc21]
or reacting compounds 2 and 5a: 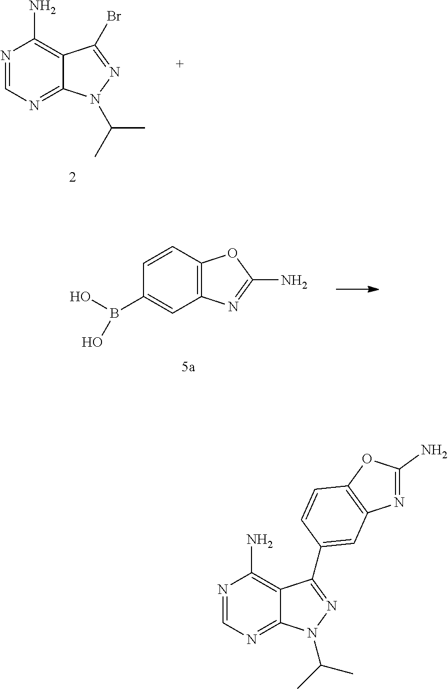 [SMILES: CC(C)n1nc(Br)c2c(N)ncnc21 ; Nc1nc2cc(B(O)O)ccc2o1 ; CC(C)n1nc(-c2ccc3oc(N)nc3c2)c2c(N)ncnc21]
to yield a compound of Formula I; and
(ii) isolating said compound of Formula I in polymorph Form A;
wherein step (ii) includes dissolving the compound of Formula I in a solvent or solvents, removing residual solid matter to yield a liquid solution, actively cooling said liquid solution at a rate to effect crystallization of Form A, and separating said Form A from the liquid solution.

16. A method of making polymorph Form A of the compound of Formula I: 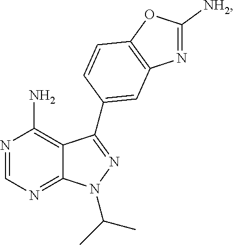 [SMILES: CC(C)n1nc(-c2ccc3oc(N)nc3c2)c2c(N)ncnc21]
said method comprising
(i) reacting compounds 2 and 5: 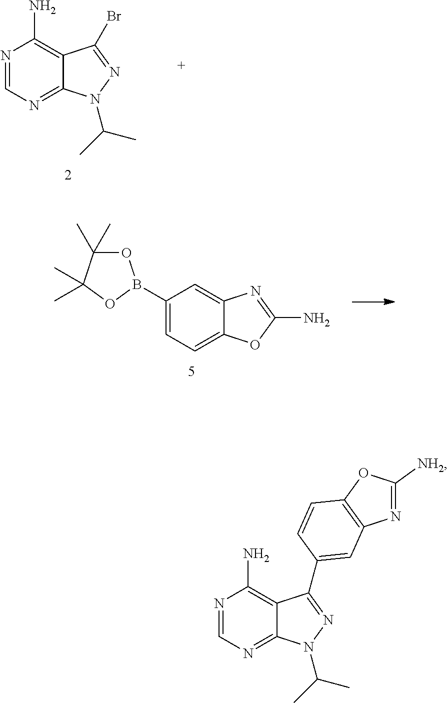 [SMILES: CC(C)n1nc(Br)c2c(N)ncnc21 ; CC1(C)OB(c2ccc3oc(N)nc3c2)OC1(C)C ; CC(C)n1nc(-c2ccc3oc(N)nc3c2)c2c(N)ncnc21]
or reacting compounds 2 and 5a: 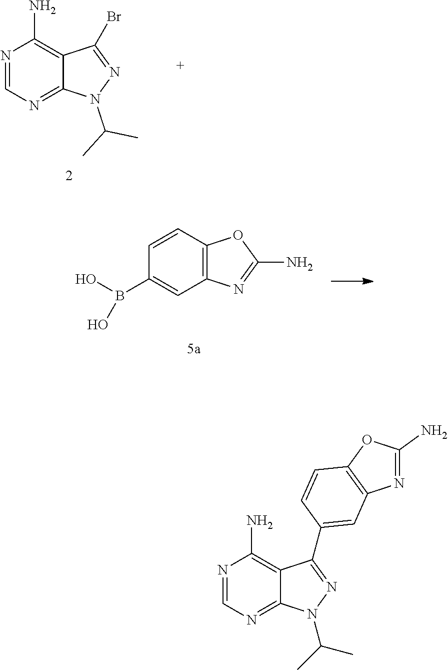 [SMILES: CC(C)n1nc(Br)c2c(N)ncnc21 ; Nc1nc2cc(B(O)O)ccc2o1 ; CC(C)n1nc(-c2ccc3oc(N)nc3c2)c2c(N)ncnc21]
to yield a compound of Formula I; and
(ii) isolating said compound of Formula I in polymorph Form A, wherein said isolation occurs under conditions to remove palladium.

17. The method of claim 16, wherein step (ii) comprises treatment of said compound of Formula I with activated charcoal.

18. The method of claim 16, wherein step (ii) comprises treatment of said compound of Formula I with methanol at reflux.

19. The method of claim 16, wherein said isolated polymorph Form A contains an amount of palladium selected from less than about 1% by weight, less than about 0.5% by weight, less than about 0.1% by weight, less than about 0.05% by weight, less than about 0.01% by weight, less than about 0.001% by weight, and less than about 0.0001% by weight.

20. A pharmaceutically-acceptable salt of the compound of Formula I: 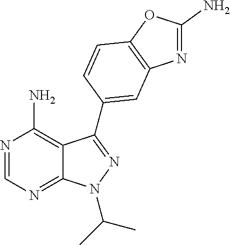 [SMILES: CC(C)n1nc(-c2ccc3oc(N)nc3c2)c2c(N)ncnc21]
and/or solvate thereof, wherein said salt is selected from L-tartaric acid, p-toluenesulfonic acid, D-glucaronic acid, ethane-1,2-disulfonic acid (EDSA), 2-naphthalenesulfonic acid (NSA), hydrochloric acid (HCl) (mono and bis), hydrobromic acid (HBr), citric acid, naphthalene-1,5-disulfonic acid (NDSA), DL-mandelic acid, fumaric acid, sulfuric acid, maleic acid, methanesulfonic acid (MSA), benzenesulfonic acid (BSA), ethanesulfonic acid (ESA), L-malic acid, phosphoric acid, and aminoethanesulfonic acid (taurine).

21. The polymorph of claim 20, wherein said compound is the HCl salt or the bis-HCl salt.

22. A composition comprising the compound of Formula I: 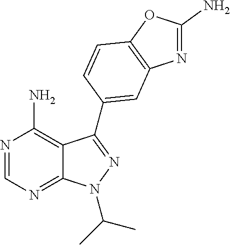 [SMILES: CC(C)n1nc(-c2ccc3oc(N)nc3c2)c2c(N)ncnc21]
or a pharmaceutically acceptable salt and/or solvate thereof,
wherein said composition comprises a mixture of polymorph Form C and one or more non-C polymorphs.

23. The composition of claim 22, wherein the composition comprises polymorph Form C and polymorph Form A.

24. The composition of claim 22, wherein the ratio of polymorph Form C to the total amount of non-C polymorphs is greater than about 1:1.

25. The composition of claim 22, wherein the ratio of polymorph Form C to the total amount of non-C polymorphs is greater than about 9:1.

26. The composition of claim 22, wherein said composition is at least 98% by weight compound of Formula I.

27. A pharmaceutical composition comprising a therapeutically effective amount of the compound of Formula I: 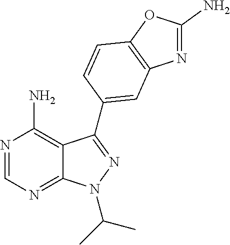 [SMILES: CC(C)n1nc(-c2ccc3oc(N)nc3c2)c2c(N)ncnc21]
or a pharmaceutically acceptable salt and/or solvate thereof, and a pharmaceutically acceptable carrier;
wherein said composition comprises polymorph Form C of the compound of Formula I.

28. The composition of claim 27, wherein said composition further comprises one or more non-C polymorphs of the compound of Formula I.

29. The composition of claim 28, wherein the ratio of polymorph Form C to the total amount of non-C polymorphs is greater than about 1:1.

30. The composition of claim 27, wherein said composition is in a solid dosage form.

31. A method for the treatment of an mTOR-associated disorder, said method comprising administering the composition of claim 27 to an individual in need thereof.

32. A compound according to the formula: 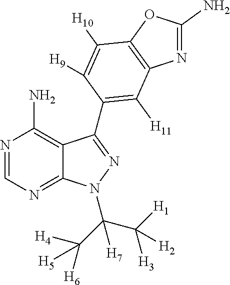
wherein at least one of H1-H11 is replaced with a deuterium atom.

33. The compound of claim 32, wherein at least one of H1-H7 is replaced with a deuterium atom.

34. The compound of claim 33, wherein each of H1-H7 is replaced with a deuterium atom.

35. A pharmaceutical composition comprising compounds of Formula I and III, 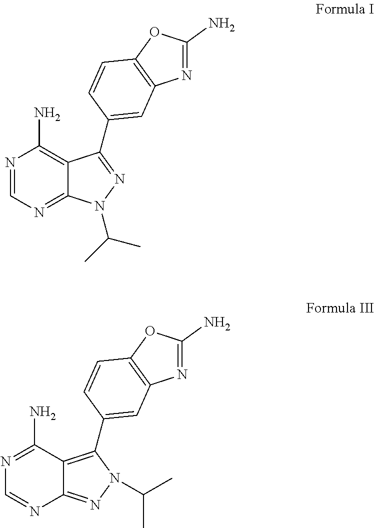 [SMILES: CC(C)n1nc(-c2ccc3oc(N)nc3c2)c2c(N)ncnc21 ; CC(C)n1nc2ncnc(N)c2c1-c1ccc2oc(N)nc2c1]
wherein the amount of compound of Formula III is less than about 50% by weight, less than about 40% by weight, less than about 30% by weight, less than about 20% by weight, less than about 10% by weight, less than about 5% by weight, less than about 4% by weight, less than about 3% by weight, less than about 2% by weight, less than about 1% by weight, less than about 0.1% by weight, or less than about 0.01% by weight, with respect to the amount of Formula I.

36. A composition comprising a hydrate or solvate of the compound of Formula I: 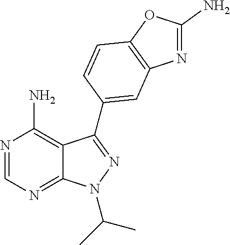 [SMILES: CC(C)n1nc(-c2ccc3oc(N)nc3c2)c2c(N)ncnc21]
and a pharmaceutically acceptable carrier.

37. The composition of claim 36, wherein the composition comprises more than one polymorph of the compound of Formula I in hydrated or solvated form.

38. The composition of claim 36, wherein the composition comprises a hydrate of Form A.

39. The composition of claim 36, wherein the composition comprises a solvate of Form A.

40. The composition of claim 39, wherein said solvate is a dimethylacetamide solvate.

41. A compound or a pharmaceutically acceptable salt of Formula I: 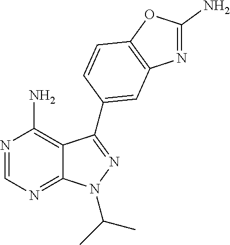 [SMILES: CC(C)n1nc(-c2ccc3oc(N)nc3c2)c2c(N)ncnc21]
wherein the compound is characterized by major peaks of XRPD diffraction pattern as shown in FIG. 1.

42. A composition consisting essentially of polymorph Form A of a compound of Formula I: 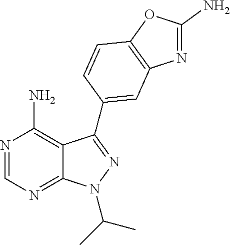 [SMILES: CC(C)n1nc(-c2ccc3oc(N)nc3c2)c2c(N)ncnc21]
and a pharmaceutically acceptable carrier.
